SEQUENCE LISTING (1 sequence)

SEQ ID NO: 1 - protein, 13 aa, synthetic construct
SIYRRGARRWRKL

(19) United States
(12) Patent Application Publication
(10) Pub. No.: US 2025/0032530 A1
(43) Pub. Date: Jan. 30, 2025

(54) USE OF AURANOFIN AS AN INHIBITOR OF ATYPICAL PROTEIN KINASE C FOR TREATMENT OF NEURODEGENERATIVE DISORDERS

(71) Applicants: UNIVERSITY OF SOUTH FLORIDA, Tampa, FL (US); THE UNITED STATES GOVERNMENT AS REPRESENTED BY THE DEPARTMENT OF VETERANS AFFAIRS, Washington, D.C., DC (US)

(72) Inventors: Robert Vito FARESE, Tampa, FL (US); Mini Paliyath SAJAN, Tampa, FL (US)

(21) Appl. No.: 18/704,415

(22) PCT Filed: Oct. 26, 2022
(86) PCT No.: PCT/US2022/078699

§ 371 Date: Apr. 24, 2024

Related U.S. Application Data
(60) Provisional application No. 63/263,021, filed on Oct. 26, 2021.

(51) Int. Cl. A61K 31/7135; A61K 9/00; A61K 31/122; A61K 31/4164

(52) U.S. Cl. CPC A61K 31/7135; A61K 9/0043; A61K 31/122; A61K 31/4164

(57) ABSTRACT

Provided herein are methods of treating or preventing a neurodegenerative disorder in a subject in need thereof, the method comprising administering an effective amount of a PKC inhibitor, wherein the PKC inhibitor is capable of crossing the blood brain barrier, and wherein the administered PKC inhibitor decreases BACE1 levels in the subject in need thereof.

### CROSS-REFERENCE TO RELATED APPLICATIONS

This application claims priority to U.S. Provisional Application No. 63/263,021 filed on Oct. 26, 2021, the content of which is incorporated by reference herein in its entirety.

### SEQUENCE LISTING

A Sequence Listing accompanies this application and is submitted as an XML file of the sequence listing named “173738.02574_Sequence Listing.xml” which is 1,855 bytes in size and was created on Oct. 25, 2022. The sequence listing is electronically submitted via Patent Center with the application and is incorporated herein by reference in its entirety.

### BACKGROUND

Alzheimer's disease (AD) is a neurodegenerative disorder that effects over 5.5 million people in the U.S. alone and over 25 million world-wide. AD and other neurodegenerative disorders are a significant public health problem with very limited therapies and no viable cures. As such, there exists an urgent and unmet need for improved therapies and/or preventatives for neurodegenerative diseases, including AD.

### SUMMARY

The present invention overcomes the aforementioned drawbacks by providing methods of administering a PKC inhibitor to subjects with a neurodegenerative disorder, for example, an aPKC inhibitor.

In a first aspect, the disclosure provides a method of treating a neurodegenerative disorder in a subject in need thereof, the method comprising administering an effective amount of a PKC inhibitor, wherein the PKC inhibitor is capable of crossing the blood brain barrier, and wherein the administered PKC inhibitor decreases BACE1 levels in the subject in need thereof.

In another aspect, the disclosure provides a method of preventing a neurodegenerative disorder in a subject in need thereof, the method comprising administering an effective amount of a PKC inhibitor, wherein the PKC inhibitor is capable of crossing the blood brain barrier, and wherein the administered PKC inhibitor decreases BACE1 levels in the subject in need thereof.

In another aspect, the disclosure provides a method of treating a neurodegenerative disorder in a subject in need thereof, the method comprising administering an effective amount of a PKC inhibitor, wherein the PKC inhibitor is capable of crossing the blood brain barrier, and wherein the administered PKC inhibitor increases a functional ability of the subject.

In a further aspect, the functional ability is determined by at least one test from the group comprising memory test, learning test, spatial memory test, object location memory test, and visual memory test.

In a further aspect, BACE1 levels are protein or RNA levels. In some aspects, protein levels are measured by Western blot or ELISA. In other aspects, RNA levels are measured by quantitative PCR. In some aspects, BACE1 levels are measured in the brain or liver. In further aspects, BACE1 levels in the subject are compared to a control sample or to a sample previously collected from the subject. In some aspects, the control sample is from a healthy subject or the subject in need thereof or is an established expression level.

In another aspect, BACE1 levels are measured after the PKC inhibitor has been administered for a duration of 1 to 31 days

In another aspect, the PKC inhibitor is an atypical PKC inhibitor. In further aspects, the atypical PKC inhibitor is selected from the group comprising auranofin, ICAP, ACPD, ICA-1, DNDA, and -Stat. In some aspects, ICAP is given at a dosage of between 1 to 5000 mg per kg of body weight.

In another aspect, the PKC inhibitor is administered intravenously, orally, nasally, or subcutaneously.

In some aspects, the neurodegenerative disorder is Alzheimer's Disease.

In some aspects, the subject has insulin resistance, obesity, metabolic syndrome, or type 2 diabetes. In a further aspect, the subject is currently taking insulin. In a further aspect, the subject has hyperinsulinemia.

In yet further aspects, the PKC inhibitor decreases proinflammatory cytokine levels. In further aspects, the proinflammatory cytokine is TNF-α or IL-6.

In another aspect, the disclosure provides a method of treating Alzheimer's Disease in a subject in need thereof, the method comprising nasally administering auranofin to the subject in need thereof.

The foregoing and other aspects and advantages of the invention will appear in the following description. In the description, reference is made to the accompanying drawings which form a part hereof, and in which there is shown by way of illustration preferred embodiments of the invention. Such embodiments do not necessarily represent the full scope of the invention, however, and reference is made therefore to the claims and herein for interpreting the scope of the invention.

### BRIEF DESCRIPTION OF THE DRAWINGS

Further aspects of the present disclosure will be readily appreciated upon review of the detailed description of its various embodiments, described below, when taken in conjunction with the accompanying drawings.

FIG. 1 shows the levels of 70 kDa PKC-λ in brains of total body heterozygous PKC-λ knockout (TB/HEtλKO) mice versus wild type (WT) mice treated acutely±intraperitoneal insulin (1 U/kg×15 min) and consuming either a regular diet (RD) or a high fat diet (HFD). (a) Representative Western blots of 70 kDa PKC-λ/ι and 50 kDa aPKC-ζ (largely PKMζ) are shown here. Unchanged levels of 50 kDa PKC-ζ and β-actin (also see FIGS. 1, 4 and 6) serve as loading controls for alterations in levels of 70 kDa PKC-λ/ι here and in FIGS. 1-6. (b) Values are mean±SEM of findings of 16-24 mice. Note that samples used here are the same as those used in FIGS. 1-6 in which there were 4-6 mice in each of the 4 treatment groups in wild type (WT) and TB/HETλKO mice. Significance: P<0.0001; TB/HETλKO vs WT (ANOVA).

FIG. 2 shows the effects of high-fat-diet (HFD) versus regular diet (RD) and effects of acute±intraperitoneal insulin treatment (1 U/kg×15 min) on brain 70 kDa PKC-λ activity, Akt activity. Aβ1-40/42 levels and phospho-thr-231-tau levels in brains of TB/HETλKO versus wild-type (WT) mice. Representative Western blots of indicated proteins are shown here. See FIGS. 1, 4 and 6 for unchanged levels of 50 kDa PKC-ζ and β actin that serve as loading controls. Bargram values are Mean±SEM of findings in 4-6 mice/group. Significance: *. P<0.05; #P<0.01; $, P<0.001; TB/HETλKO versus similarly treated and fed WT group; (ANOVA).

FIG. 3 shows the effects of high-fat diet (HFD) (+) versus regular diet (−) and effects of acute intraperitoneal insulin (Ins) treatment (1 U/kg×15 min) on brain levels of: (a) Aβ40 peptide; (b) Aβ42 peptide; (c) secreted/soluble fragment of βAPP (sAPPα) generated by α-secretase action; and (d) the 99 amino acid C-terminal fragment of βAPP (CTFO) generated by BACE1 action, in TB/HETλKO mice versus wild-type (WT) mice. Values are mean±SEM of findings in 4-6 mice/group. Significance: *, p<0.05, indicated WT mice versus WT mice on regular diet and not treated with Ins; $, p<0.05, indicated TB/HETλKO (KO) mice versus similarly treated and fed WT mice (ANOVA).

FIG. 4 shows the effects of high-fat (HF) diet (+) versus regular diet (−) and acute intraperitoneal insulin treatment (1 U/kg×15 min) on brain levels of BACE1 protein in TB/HETλKO versus wild-type (WT) mice. (a) Representative Western blots. (b) Values are mean SEM of 4-6 mice/group. Significance: *, P<0.05, before WT RD; †, P<0.001, vs WT RD; ‡, P<0.01, vs TB/HetλKO RD; !, P<0.01, vs TB/HetλKO RD+Ins; §, P<0.01, vs TB/HetλKO HF (ANOVA).

FIG. 5 shows the effects of high-fat diet (HFD) versus regular diet (RD) and acute intraperitoneal insulin treatment (1 U/kg×15 min) on levels of BACE1 mRNA in brains of TB/HETλKO (KO) mice versus wild-type (WT) mice. Values are mean±SEM of 4-6 mice/group. Significance: *, p<0.01 vs WT/RD-Ins or WT/HFD-Ins; t, p<0.01 vs WT/RD-Ins or WT/HFD-Ins; #, p<0.01 vs KO/RD-Ins or KO/HFD-Ins; (ANOVA).

FIG. 6 shows the effects of high-fat diet (HFD) versus low-fat regular diet and effects of acute intraperitoneal insulin (Ins) (+) (1 U/kg×15 min) or vehicle (−) treatment on: (A) NFκB/p65RelA activity; (B) IKKα/β activity; (C) TNF-α levels; and (D) IL-6 levels in brains of TB/HETλKO mice (shaded bars) and littermate wild-type (WT) mice (solid bars). Shown in A and B are representative blots of phosphorylated/activated forms of NFκB/p65RelA and IKKα/β in upper bands and R-actin loading controls in lower bands. TNF-α and IL-6 levels were measured by ELISA. Values are mean±SEM of findings in 4-6 mice/group. Asterisks indicate a significance of p<0.001 for comparison of TB/HETλKO versus WT mice in corresponding (adjacent) diet/treatment groups (ANOVA).

FIG. 7 shows the radial-arm water maze (RAWM) (left) and Object Location Memory (OLM) (right) testing of working memory in wild-type (WT) and TB/HETλKO mice. Values are mean SEM of 7-9 mice/group in RAWM tests and 11-13 mice/group in OLM tests. There were no significant differences between groups (ANOVA).

FIG. 8 shows the effects of nasally-administered 1H-imidazole-4-carboxamide, 5-amino-1-[2,3-dihydroxy-4-[(hydroxyl)methyl]cyclopentyl-[1R(1a,2b, 3b,4a) (ICAP)](100 mg/kg/day×21 days) on brain and liver 70 kDa and 50 kDa aPKC activities (p-thr-555/560-aPKC), and levels of total aPKC, BACE1 and β-actin (loading control) in normal mice. Values are mean±SEM of 6 mice/group. P values, (ANOVA).

FIG. 9 shows the effects of intravenously (IV)-administered 1H-imidazole-4-carboxamide, 5-amino-1-[2,3-dihydroxy-4-[(hydroxyl)methyl]cyclopentyl-[1R-(1a,2b, 3b,4a)] (ICAP) (100 mg/kg/day×7 days) on brain and liver 70 kDa and 50 kDa aPKC activities (pthr-555/560-aPKC), and levels of BACE1 and R-actin (loading control) in normal mice. Values are mean±SEM of 6 mice/group. Asterisks: P<0.001 (ANOVA).

FIG. 10 shows the dependence of insulin-induced activation of NFκB on PKC-ι/λ in isolated neuronal cells. Human neuroblastoma-derived LA1-5s cells were treated with 200 nM insulin±aPKC inhibitors, 1 μM ACPD or increasing concentrations of Auranofin (AF), for 24 hours. Cell lysates were subjected to Western analyses for phosphorylated/activated forms of aPKC, NFκB and Akt. Representative blots of indicated proteins are shown in upper bands. Lower bands show GAPDH loading controls. Bargrams show Mean±SEM of 3-4 determinations. Asterisks indicate P<0.05; comparison of designated group versus the uninhibited, insulin-treated group (ANOVA).

FIG. 11 shows the effects of 24-hour treatment with 200 nM insulin±increasing concentrations of ICAPP on BACE1 levels in cultures of LA1-5s human neuroblastoma-derived neuronal cells. Values of BACE1 are Mean±SEM of 4 values. Asterisks indicate P<0.05 for comparison of designated group versus the untreated control group (ANOVA).

FIG. 12 shows CNS Signaling pathways in Neurodgenerative Processes.

FIG. 13 shows depletion of PKC-λ in total body (TB) heterozygous KO mice and neuronal homozygous KO mice. Blots of 2 mice/group are shown here and are representative of findings in 10 mice in each group.

FIG. 14 shows decreased production of Aβ-peptides (1-40 and 1-42) (left and center panels) and deposition of Aβ-plaque (right panel) in Tg APP/PS1 AD mice following total body heterozygous PKC-λ KO (het) and neuron-specific homozygous (KO) knockout of PKC-λ. Brain Aβ-peptides were measured by ELISA. Plaques in brain halves were counted microscopically. Results are Mean±SEM of 10 mice/group. Symbols indicate: (*), P<0.001, APP/PS-1 P het mice vs. APP/PS-1 mice; and (†), P<0.001, APP/PS1 KO mice vs. APP/PS-1 he mice; (ANOVA).

FIG. 15 shows decreased levels of CD-68 staining-positive microglial cells (left), GFAP staining-positive astroglial cells (middle panel), and proinflammatory cytokines in brains of Tg APP/PS1 AS mice following total body heterozygous PKC-λKO (het) and neuron-specific homozygous (KO) knockout of PKC-λ. Cytokine levels (TNF-α, IL-1β) were measured by ELISA. Results are Mean±SEM of 10 mice/group. Symbols indicate: (*), P<0.001, APP/PS-1 P het mice vs. APP/PS-1 mice; and (t), P<0.001, APP/PS1 KO mice vs. APP/PS-1 he mice; (ANOVA).

FIG. 16 shows improvements in learning and memory functions in Radial Arm Water Maze Test in Tg APP/PS1 AD mice following total body heterozygous PKC-λ KO (het) and neuron-specific homozygous (KO) knockout of PKC-λ. Results are Mean±SEM of 10 mice/group. Symbols indicate: (*), P<0.001, APP/PS-1 P het mice vs. APP/PS-1 mice; and (t), P<0.001, APP/PS1 KO mice vs. APP/PS-1 he mice; (ANOVA).

FIG. 17 shows activation of PKC-λ/ι in transgenic APP/PS1 AD mice and reversal by oral ICAP (100 mg/kg/day) Rx×3 mos. Representative data of 10 mice/group. *, P<0.001 vs WT. †, P<0.001 vs −ICAP.

FIG. 18 shows effects of oral ICAP treatment (100 mg/kg body weight/day×3 mos) of transgenic APP/PS1 AD mice on levels of Aβ-peptides (as per ELISA). Data of 10 mice/group. *, P<0.001 vs WT. †, P0.001 vs −ICAP.

FIG. 19 shows effects of oral ICAP treatment (100 mg/kg body weight/day×3 mos) of transgenic APP/PS1 AD mice on levels of AD-plaque load (left) and relative discrimination index in Novel Object Recognition memory testing (right). Data of 10 mice/group. *, P<0.001 vs WT. †, P0.001 vs −ICAP (ANOVA).

FIG. 20 shows decreases in levels of BACE1 (left) and proinflammatory cytokines (right) in brains of APP/PS1 AD mice following treatment with daily ICAP treatment (100 mg/kg-bw) given by oral gavage (PO). Liver BACE1 levels were also diminished by this PO ICAP treatment. Values are Mean±SEM of results in 10 mice.

FIG. 21 shows effects of PGRN and aPKC inhibitor ACPD on insulin-stimulated activation of IRS-1, PKC-λ/ζ, NF-κB/p65RelA, IRS-2, and Akt in LA1-5s neural cells. Cells were incubated for 24 hours±200 nM insulin, 500 ng/mL PGRN, and 1 μM ACPD, as indicated. Values are Mean±SEM (N=4-6). Asterisks indicate p<0.05 vs insulin-stimulated values and † indicates p<0.001 vs unstimulated control value (ANOVA). Total protein levels were unchanged by treatments. CON=control; INS=insulin; LC=loading control (lower bands).

FIG. 22 shows effects of PGRN and aPKC inhibitor ACPD on insulin-stimulated activation of IRS-1, PKC-λ/ζ, NF-κB/p65ReIA, IRS-2, and Akt in HMC-3 microglial cells. Cells were incubated for 24 hours±200 nM insulin, 500 ng/mL PGRN, and 1 μM ACPD, as indicated. Values are Mean±SEM (N=4-6). Asterisks indicate p<0.05 vs insulin-stimulated values and t indicates p<0.001 vs unstimulated control value (ANOVA). Total protein levels were unchanged by treatments. CON=control; INS=insulin; LC=loading control (lower bands).

FIG. 23 shows activation of IRS-1, Akt, PKC-λ/ζ, NFκB.p65ReIA, IKKu/p, and loading control in brain, liver, skeletal muscle and fat in wild type (WT) mice and heterozygous (HET) and homozygous (HOM) PGRN knockout (KO) mice. Mice were fed ad lib and killed between 8-10 AM. Representative blots of 3-5 mice group are shown.

FIG. 24 shows effects of heterozygous (HET) and homozygous (HOM) knockout (KO) of PGRN on activities of IRS-1, PKC-λ/ζ, IKKA/B, NFκB/p86ReIA, IRS-1, Akt, mTOR, and IRP levels in the cerebral cortices of 2-3-month-old, overnight-fasted (gray bars) and 4-6-month-old, adlib-fed (black bars) WT, HET-KO, and HOM-KO littermate mice. Values are Mean±SEM of 4-6 mice. Asterisks indicate p<0.05 vs corresponding fasted WT or fed WT values (ANOVA). Note that all values in older fed mice were approx. 20-30% higher than values in younger fasted mice, but relative differences (HET-KO and HOM-KO) and corresponding WT mice were similar. For simplicity, as there were no differences, total levels of IRS-1, IRS-2, PKC-λ/ζ, IKKA/B, and NκB/p65ReIA are not shown. LC=loading control (lower band).

FIG. 25 shows effects of aPKC inhibition on high-fat-diet (HFD)-induced alteration in BACE1 and IR-β levels in brain. Mice were fed a regular diet (RD) or a 60%-fat-kcal HFD for 10 weeks and treated±aPKC inhibitors ACPD (20 mg/kg-bw QOD). GAPDH loading controls were comparable in all diet and treatment groups (not shown). Values are Mean±SEM of results from 4-6 mice. Asterisks indicate P<0.05 (ANOVA).

### DETAILED DESCRIPTION

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a dosage range is stated as 1 to 5000 mg/kg, it is intended that values such as 2 mg/kg to 100 mg/kg, 10 mg/kg to 30 mg/kg, or 1 mg/kg to 3 mg/kg, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed. As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

The following examples are meant only to be illustrative and are not meant as limitations on the scope of the invention or of the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

Embodiments of the present disclosure will employ, unless otherwise indicated, techniques of molecular biology, microbiology, nanotechnology, organic chemistry, biochemistry, endocrinology, physiology, and the like, which are within the skill of the art. Such techniques are explained fully in the literature.

The present invention is based on work by the inventors demonstrating that β-amyloid precursor protein-cleaving enzyme-1 (BACE1) initiates the production of Aβ-peptides that form Aβ-plaque in Alzheimer's disease. The inventors found that total-body heterozygous PKC-λ knockout reduced acute stimulatory effects of insulin and chronic effects of hyperinsulinemia in HFF/obese/diabetic mice, on brain PKC-λ activity and production of Aβ1-40/42 and phospho-thr-231-tau. In addition, heterozygous knockout of PKC-λ markedly reduced brain levels of BACE1 protein and mRNA and may reflect diminished activation of nuclear factor kappa-B (NFκB), which is activated by PKC-λ and increases BACE1 and proinflammatory cytokine transcription. Accordingly, whereas intravenous administration of aPKC inhibitor diminished aPKC activity and BACE1 levels by 50% in the brain and 90% in the liver, nasally administered inhibitor reduced aPKC activity and BACE1 mRNA and protein levels by 50-70% in the brain while sparing the liver. Additionally, 24-hour insulin treatment in cultured human-derived neurons increased NFκB activity and BACE1 levels, and these effects were blocked by various PKC-λ/ι inhibitors. PKC-λ/ι inhibitors may be used nasally to target brain PKC-λ/ι or systemically to block both liver and brain PKC-λ/ι, to regulate NFκB-dependent BACE1 and proinflammatory cytokine expression.

In some embodiments, the present disclosure provides a method of treating a neurodegenerative disorder in a subject in need thereof, the method comprising administering an amount of a PKC inhibitor capable of crossing the blood brain barrier to the subject in need thereof, wherein the amount of the PKC inhibitor is an amount effective to decrease BACE1 levels.

In some embodiments, the present disclosure provides a method of preventing a neurodegenerative disorder in a subject in need thereof, the method comprising administering an amount of a PKC inhibitor capable of crossing the blood brain barrier to the subject in need thereof, wherein the amount of the PKC inhibitor is an amount effective to decrease BACE1 levels.

In some embodiments, the present disclosure provides a method of treating a neurodegenerative disorder in a subject in need thereof, the method comprising administering an amount of a PKC inhibitor capable of crossing the blood brain barrier to the subject in need thereof, wherein the amount of the PKC inhibitor is an amount effective to increase a functional ability of the subject.

In some embodiments, the method(s) can include detecting protein or RNA expression levels using Western blot, ELISA, or qPCR, measuring expression levels in the brain or liver, comparing expression levels to a control, administering the PKC inhibitor for a duration of 1 to 31 days, administering an atypical PKC inhibitor, administering the PKC inhibitor at a dose of 1 to 5000 mg per kg of body weight, administering the PKC inhibitor nasally, treating subjects with Alzheimer's disease, treating subjects with metabolic syndrome(s), and/or decreasing proinflammatory cytokine levels.

Alzheimer's disease (AD) is characterized by increases in brain levels of potentially neurotoxic Aβ-peptides (Aβ) that aggregate to form pathological plaques with associated increases in neurofibrillary tangles of phospho-tau. Increases in Aβ-peptides are initiated by aspartyl-peptidase action of neuronal β-secretase [β-site amyloid precursor protein (APP)-cleaving enzyme-1 (BACE1)] on βAPP, followed by γsecretase action.

Some, but not all, studies have observed increases in brain levels and/or activity of BACE1 in sporadic non-familial late-onset AD. However, Aβ production is complex and may also involve alterations in trafficking and interactions between BACE1-containing and βAPP-containing vesicles, and Aβ levels can be altered by changes in AR clearance and actions of α-secretase, which cleaves βAPP within the Aβ sequence, thus precluding the production of full-length Aβ-peptides. Irrespective of whether BACE1 levels are altered in various forms of AD, BACE1 action is critical for producing increases in Aβ, and possibly phospho-tau. Regardless of how increases in Aβ and phospho-tau are produced, these pathologic aberrations are thought to cause impairments in hippocampal-dependent learning and memory functions eventually leading to neurodegenerative pathological processes throughout the brain.

AD-related elevations in brain BACE1 activity or levels have been attributed to a variety of factors, perhaps most notably, those involving oxidative and inflammatory stress. In this regard, it appears that BACE1 expression in the brain and perhaps other tissues is mainly controlled by the master regulator of inflammation, nuclear factor kappa-B (NFκB), which also controls the expression of proinflammatory cytokines, e.g., tumor necrosis factor-α (TNF-α), interleukin 1-β (IL-1β), and IL-6. This inter-relationship between BACE1 and proinflammatory cytokines raises the possibility that various AD risk factors may converge at a single definable signaling pathway that leads to NFκB activation and development of AD pathology due to increases in levels of BACE1 and proinflammatory cytokines.

### Definitions

As used herein, “about,” “approximately,” and the like, when used in connection with a numerical variable, generally refers to the value of the variable and to all values of the variable that are within the experimental error (e.g., within the 95% confidence interval for the mean) or within +10% of the indicated value, whichever is greater.

As used herein, “administering” refers to an administration that is oral, nasal, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, or via an implanted reservoir. The term “parenteral” includes subcutaneous, intravenous, intramuscular, intra-articular, intra-synovial, intrastemal, intrathecal, intrahepatic, intralesional, and intracranial injections or infusion techniques.

As used herein, “active agent” or “active ingredient” refers to a component or components of a composition to which the whole or part of the effect of the composition is attributed.

As used herein, “anti-infectives” can include, but are not limited to, antibiotics, antibacterials, antifungals, antivirals, and antiproatozoals.

As used herein, a “PKC inhibitor” refers to a compound that can reduce the amount of and/or activity of a PKC. The PKC inhibitor can be a specific PKC inhibitor, which includes a PKC inhibitor that acts on a PKC and does not have any measurable or significant action on other PKCs. Exemplary PKC inhibitors include Calphostin C (UCN-1028C), 2,6-Diamino-N-([1-oxotridecyl)-2-piperidinyl]methyl)hexanamide (NPC 15437), N-benzyladriamycin-14-valerate, safingol (L-threo-dihydrosphingosine), resveratrol, Staurosporine, 7-hydroxystaurosporine (UCN-01), 4′-N-benzoylstaurosporine (Midostaurin), enzastaurin (LY317615), ruboxistaurin (LY 333531), sotrastaurin (AEB071), Balanol (SPC 100840), chelerythrine, and riluzole.

Alternatively, the PKC inhibitor can inhibit atypical PKCs (aPKC). In the case of aPKC inhibition, the inhibitor could alternatively be called an “aPKC inhibitor”.

In some embodiments, the PKC inhibitor is an atypical PKC inhibitor. Exemplary aPKC inhibitors include, but are not limited to, 1H-imidazole-4-carboxamide,-5-amino-1-[2,3-dihydroxy-4-[(hydroxyl)methyl]cyclopentyl-[1R-(1a,2b,3b,4a)] (ICAP), [1H-imidazole-4-carboxamide,5-amino]-[2,3-dihydroxy-4-[(phosphono-oxy)methyl]cyclopentane-[1R-(1a,2b,3b,4a)] (ICAPP), 2-acetyl-cyclopentane-1,3-dione (ACPD), 1-thio-,6-D-glucopyranosatotriethylphosphine gold-2,3,4,6-tetraacetate (auranofin), ZIP (SIYRRGARRWRKL; SEQ ID NO: 1), [4-(5-amino-4-carbamoylimidazol-1-yl)-2,3-dihydroxycyclopentyl] methyl dihydrogen phosphate (ICA-1), 8-hydroxy-1,3,6-naphthalenetrisulfonic acid (ζ-Stat), and 3,4-diaminonaphthalene-2,7-disulfonic acid (DNDA).

As used herein, “control” is an alternative subject or sample used in an experiment for comparison purposes and included to minimize or distinguish the effect of variables other than an independent variable. A control can be positive or negative. The control can also be an established level of expression based off healthy or unhealthy population statistics, for example. One of ordinary skill in the art will appreciate suitable controls.

As used herein, “sample”, “biological sample” or “test sample” refers to any sample of tissue, fluid, or material derived from a living organism. In some embodiments, the living organism is a primate. In some embodiments, the living organism is a human being, or Homo sapiens. Exemplary biological samples include, but are not limited to, a tissue sample, for example, a biopsy sample, or a blood sample.

Suitable methods of determining the expression of BACE1 in a subject are known and understood in the art. In one embodiment, the expression is measured by nucleic acid expression, e.g., gene expression or mRNA expression. In another embodiment, the expression is measured by protein expression in a sample extracted from the subject. Suitable methods and reagents for these methods are known in the art. For example, suitable methods to measure expression levels of DNA/RNA, include, but are not limited to, Northern blot analysis, nuclease protection assays (NPA), in situ hybridization, reverse transcription-polymerase chain reaction (RT-PCR), qRT-PCR, RNA-Seq, among others. Suitable methods to measure protein levels include, for example, immunohistochemistry, immunofluorescence, flow cytometry, mass spectroscopy, enzyme-linked immunosorbent assays (ELISA), quantitative ELISA, Western blotting, and dot blotting, among others.

As used herein, “functional ability” refers to the subject's ability to perform cognitive functions. Suitable methods for determining functional ability include memory, learning, spatial memory, object location memory, and visual memory tests. Other tests suitable for testing functional ability include, but are not limited to, those requiring the function of the hippocampal and frontal cortical areas of the brain.

Expression levels or functional ability in response to aPKC inhibitor administration may be determined after 1 day to 31 days. For example, expression levels or functional ability in response to aPKC inhibitor administration are determined after 1, 2, 3, 4, 5, 6, 7, 8, 9, 10, 20, or 30 days. In some embodiments, expression levels or functional ability in response to aPKC inhibitor administration are determined after 1 day. In other embodiments, expression levels or functional ability in response to aPKC inhibitor administration are determined after 21 days. A skilled artisan will understand, within the context of a neurodegenerative disease such as Alzheimer's Disease, the variety and type of functional tests that can be performed on a subject to ascertain improvement in functional ability during or after a course of treatment, e.g., such as administration of a PKC inhibitor as described herein. In some embodiments, the functional ability of the subject is tested before administration of a PKC inhibitor. In some embodiments, this is considered a base level, or control level of functional ability. In some embodiments, a subject is administered a PKC inhibitor, as a single administration, or as a course of therapy. By way of example, in some embodiments a subject is administered a PCK inhibitor weekly, twice per week, three times per week, four times per week, five time per week, or daily (e.g., once per day, twice per day, three times per day, four or more times per day) over the course of one week, two weeks, three weeks, one month, two months three months, four months, five months, six months, seven months, eight months, nine months, ten months, eleven months, or daily for one year or more. In some embodiments, the subject is administered a PKC inhibitor as needed, based on symptoms, e.g., functional ability. By way of example, with improved functional ability, in some embodiments, dosage may be decreased. In some embodiments, the subject's functional ability is tested before, during or after a course of treatment. In some embodiments, the subject exhibits a statistically significant improvement in functional ability after 1 week, 2 weeks, three weeks, four weeks, five weeks, six weeks, seven weeks, two, three, four, five, six, seven, eight, nine, ten, or eleven months, or after one or more years of treatment. In some embodiments, the subject exhibits a statistical improvement in functional ability after 1-5 days of treatment, 1-3 days of treatment or after about a week of treatment. In some embodiments, the subject is administered a PKC inhibitor nasally, and in some embodiments, the PCK inhibitor comprises or consists of auranofin.

The terms “protein,” “peptide,” and “polypeptide” are used interchangeably herein and refer to a polymer of amino acid residues linked together by peptide (amide) bonds. The terms refer to a protein, peptide, or polypeptide of any size, structure, or function. The terms “nucleic acid” and “nucleic acid molecule,” as used herein, refer to a compound comprising a nucleobase and an acidic moiety, e.g., a nucleoside, a nucleotide, or a polymer of nucleotides. Nucleic acids generally refer to polymers comprising nucleotides or nucleotide analogs joined together through backbone linkages such as but not limited to phosphodiester bonds. Nucleic acids include deoxyribonucleic acids (DNA) and ribonucleic acids (RNA) such as messenger RNA (mRNA), transfer RNA (tRNA), etc.

As used herein, “dose,” “unit dose,” or “dosage” refers to physically discrete units suitable for use in a subject, each unit containing a predetermined quantity of the composition or formulation calculated to produce the desired response or responses in association with its administration.

As used herein, “effective amount” or “amount effective”, as used interchangeably herein, can refer to the amount of a composition or pharmaceutical formulation described herein that will elicit a desired biological or medical response of a tissue, system, animal, plant, protozoan, bacteria, yeast, or human that is being sought by the researcher, veterinarian, medical doctor or other clinician. The effective amount will vary depending on the exact chemical structure of the composition or pharmaceutical formulation, the causative agent and/or severity of the infection, disease, disorder, syndrome, or symptom thereof being treated or prevented, the route of administration, the time of administration, the rate of excretion, the drug combination, the judgment of the treating physician, the dosage form, and the age, weight, general health, sex and/or diet of the subject to be treated. “Effective amount” can refer to an amount of a composition or pharmaceutical formulation described herein that can reduce BACE1 levels, prevents the increase of BACE1 levels, or reduce functional impairment, such as in the CNS, directly and/or indirectly decrease activity of aPKC in the CNS, decrease activity of NF-κB in the CNS, decrease the amount of Aβ_{1-40/42} in the CNS, decrease the amount of thr-231-phospho-tau in the CNS, decrease the activity of aPKC isoform PKC-λ/ι in the CNS, and/or decrease proinflammatory cytokine levels in the CNS including, but not limited to, TNF-α or IL-6. The “effective amount” can refer to the amount of an aPKC inhibitor or formulation thereof described herein that can treat and/or prevent a neurodegenerative disorder or a symptom thereof. Such neurodegenerative disorders can be, without limitation, Alzheimer's Disease.

As used herein, “neurodegenerative disorders” are hereditary or sporadic conditions which are characterized by progressive nervous system dysfunction. These disorders are often associated with atrophy of the affected central or peripheral structures of the nervous system. Non-limiting examples of neurodegenerative disorders include Alzheimer's disease and other dementias, degenerative nerve diseases, genetic brain disorders, Parkinson's disease, amyotrophic lateral sclerosis (ALS or Lou Gehrig's disease), Huntington's disease, and prion diseases.

As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo.

As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient.

As used herein, “pharmaceutically acceptable salt” refers to any acid or base addition salt whose counter-ions are non-toxic to the subject to which they are administered in pharmaceutical doses of the salts.

As used interchangeably herein, “subject,” “individual,” or “patient,” refers to a vertebrate, preferably a mammal, more preferably a human. Mammals include, but are not limited to, rodents, simians, humans, farm animals, sport animals, and pets. The term “pet” includes a dog, cat, guinea pig, mouse, rat, rabbit, ferret, and the like. The term farm animal includes a horse, sheep, goat, chicken, pig, cow, donkey, llama, alpaca, turkey, and the like.

The terms “treat,” “treating,” “treatment,” and grammatical variations thereof as used herein include partially or completely delaying, alleviating, mitigating or reducing the intensity of one or more attendant symptoms of a disorder or condition such as neurodegenerative disorder and/or alleviating, mitigating or impeding one or more causes of a disorder or condition such as a neurodegenerative disorder. Treatments according to the embodiments disclosed herein may be applied preventively, prophylactically, palliatively, or remedially, which are collectively referred to herein as “preventing”. In some instances, the terms “treat,” “treating,” “treatment,” and grammatical variations thereof include partially or completely reducing a condition or symptom associated with a neurodegenerative disorder prior to treatment of the subject or as compared with the incidence of such condition or symptom in a general or study population.

### DISCUSSION

Neurodegenerative disorders, such as Alzheimer's Disease (AD), are a significant public health issue directly affecting over 25 million people worldwide. The pathologies of some neurodegenerative disorders are poorly understood. Additionally, there are limited treatment options for neurodegenerative disorders, which mainly focus on management of the disorder and maintaining quality of life as opposed to treating the underlying pathology.

Moreover, what treatments are available that directly combat the disorder often come with harsh side effects due to their non-specificity.

It has been observed that some neurodegenerative disorders, such as AD, occurs at a higher rate in patients with other diseases or disorders, such as diabetes or metabolic syndrome. Although it was previously thought that this was a side effect of the brain being insulin resistant, this does not appear to be the case in at least some instances. Atypical PKC (aPKC), can be hyperactivated in the liver and the brain by hyperinsulinemia, which can directly or indirectly lead to aberrant signaling in the brain. Aberrant signaling in the brain can contribute to the development of neurodegenerative disorders.

Described herein are aPKC inhibitors and formulations thereof that can directly or indirectly decrease aPKC in the brain and methods of treating a neurodegenerative disorder, such as AD, in a subject in need thereof by administering an aPKC inhibitor or formulation thereof to the subject in need thereof. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

aPKC Inhibitors and Pharmaceutical Formulations Thereof

Insulin can result in an increase in atypical PKC (aPKC) activity in the liver, muscle, and brain. Increased and hyperactivity of aPKC can negatively impact signaling the periphery and central nervous system (CNS), which can cause and/or potentiating disorders such as metabolic syndrome, diabetes, and insulin resistance in the periphery and aberrant signaling in the CNS. Inhibition of aPKC directly in the CNS can reduce hyperinsulinemia and/or the effects thereof and restore signaling in the CNS. Inhibition of aPKC and/or Akt activity in the periphery (e.g. liver and/or muscle) can indirectly reduce the effects of hyperinsulinemia in the brain as a result of restoring insulin levels to normal in the periphery and CNS, which can result in a restoration of signaling to normal in the CNS. Provided herein are aPKC inhibitors and pharmaceutical formulations thereof that can be used for treatment and/or prevention of a neurodegenerative disorder, such as Alzheimer's disease (AD), in a subject in need thereof. The aPKC inhibitor can be capable of crossing the blood brain barrier (that is, the highly selective semipermeable border of endothelial cells that prevents solutes in the circulating blood from non-selectively crossing into the extracellular fluid of the central nervous system where neurons reside) and thus directly inhibit aPKC in the CNS of the subject. In some aspects, the aPKC inhibitor that can be capable of crossing the blood brain barrier can be 1H-imidazole-4-carboxamide,-5-amino-1-[2,3-dihydroxy-4-[(hydroxyl)methyl]cyclopentyl-[1R-(1a,2b,3b,4a)] (ICAP), [1H-imidazole-4-carboxamide,5-amino]-[2,3-dihydroxy-4-[(phosphono-oxy)methyl]cyclopentane-[1R-(1a,2b,3b,4a)] (ICAPP), 2-acetyl-cyclopentane-1,3-dione (ACPD), or 1-thio-β-D-glucopyranosatotriethylphosphine gold-2,3,4,6-tetraacetate (auranofin). In other aspects, the aPKC inhibitor cannot cross the blood brain barrier, and thus when administered systemically can indirectly inhibit aPKC activity and/or amount in the CNS by altering (such as decreasing) circulating, and thus CNS, insulin levels which can decrease the activity of CNS aPKC. The aPKC inhibitor that cannot cross the blood brain barrier can be aurothiomalate (ATM).

Auranofin (Ridaura; 1-thio-β-D-glucopyranosatotriethylphosphine gold-2,3,4,6-tetraacetate) exhibit immunosuppressive activity and have been used in the treatment of rheumatoid arthritis (Bruckle, et al., 1994; Jessop, et al., 1998). It also inhibits PKCι and PKCζ signaling by selectively targeting the PB1 domain (Cys-69) of PKCι (Erdogan, et al., 2006) and the PB1 domain (Cys-68) of PKCι, respectively (Butler, et al., 2015). The binding of auranofin to the PB1 domain blocks the interaction of PKCι and PKCζ with their adaptors, Par6, p62, and MEK5 (Erdogan, et al., 2006; Butler, et al., 2015). The cytotoxicity activity (IC₅₀) of auranofin is ≤10 μM in sensitive cancer cells and >40 μM in non-sensitive cancer cells (Li, et al., 2016; Mirabelli, et al., 1986; Regala, et al., 2008).

A feasibility study for enrolling asymptomatic ovarian cancer patients with increased levels of CA-125 (10 patients) has also been carried out by oral administration of auranofin, which resulted in decreased levels of CA-125 in one patient (Jatoi, et al., 2015). Furthermore, a phase I/II clinical trial of auranofin (NCT01419691) has been conducted in patients with chronic lymphocytic leukemia, small lymphocytic and prolymphocytic lymphoma (Kambhampati, et al., 2016).

### Pharmaceutical Formulations

Also provided herein are pharmaceutical formulations that can include an amount of an aPKC inhibitor described herein and a pharmaceutical carrier appropriate for administration to an individual in need thereof. The individual in need thereof can have or can be suspected of having a neurodegenerative disorder or symptom thereof. The subject in need thereof can also have hyperinsulinemia in the periphery and/or CNS, type 1 diabetes, type 2 diabetes, obesity, metabolic syndrome, and/or a symptom thereof. The pharmaceutical formulations described herein can include an amount of an aPKC inhibitor described herein that can be an amount effective to treat and/or prevent a neurodegenerative disorder or a symptom thereof. In some aspects the neurodegenerative disorder is AD. The aPKC inhibitor, in some aspects, can be included in the manufacture of a medicament for treatment of a neurodegenerative disorder or a symptom there, including, but not limited to, AD.

The formulations provided herein can be administered via any suitable administration route. For example, the formulations (and/or compositions) can be administered to the subject in need thereof orally, intravenously, intramuscularly, intravaginally, intraperitoneally, rectally, parenterally, topically, intranasally, or subcutaneously. Other suitable routes are described herein.

### Parenteral Formulations

The aPKC inhibitor can be formulated for parenteral delivery, such as injection or infusion, in the form of a solution or suspension. The aPKC inhibitor contained in the pharmaceutical formulation can be capable of crossing the blood brain barrier when administered systemically. In some aspects, the aPKC inhibitor or amount thereof that is capable of crossing the blood brain barrier can be effective to reduce the activity and/or amount of periphery (e.g. liver and muscle) and/or CNS (e.g. neuronal and brain) aPKC. The aPKC inhibitor that can be capable of crossing the blood brain barrier can be ICAPP or ACPD or a pharmaceutical salt. In other aspects, the aPKC inhibitor is not capable of crossing the blood brain barrier. Thus, in some aspects, the aPKC inhibitor that is not capable of crossing the blood brain barrier can be ATM or a pharmaceutical salt thereof. The formulation can be administered via any route, such as, the blood stream or directly to the organ or tissue to be treated.

Parenteral formulations can be prepared as aqueous compositions using techniques is known in the art. Typically, such compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsomes.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride.

Solutions and dispersions of the aPKC inhibitor as described herein can be prepared in water or another solvent or dispersing medium suitably mixed with one or more pharmaceutically acceptable excipients including, but not limited to, surfactants, dispersants, emulsifiers, pH modifying agents, and combination thereof.

Suitable surfactants can be anionic, cationic, amphoteric, or nonionic surface active agents. Suitable anionic surfactants include, but are not limited to, those containing carboxylate, sulfonate and sulfate ions. Suitable anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Suitable cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Suitable nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-β-alanine, sodium N-lauryl-β-iminodipropionate, myristoamphoacetate, lauryl betaine and lauryl sulfobetaine.

The formulation can contain a preservative to prevent the growth of microorganisms. Suitable preservatives include, but are not limited to, parabens, chlorobutanol, phenol, sorbic acid, and thimerosal. The formulation can also contain an antioxidant to prevent degradation of aPKC inhibitor.

The formulation can be buffered to a pH of 3-8 for parenteral administration upon reconstitution. Suitable buffers include, but are not limited to, phosphate buffers, acetate buffers, and citrate buffers.

Water-soluble polymers can be used in the formulations for parenteral administration. Suitable water-soluble polymers include, but are not limited to, polyvinylpyrrolidone, dextran, carboxymethylcellulose, and polyethylene glycol. Sterile injectable solutions can be prepared by incorporating the aPKC inhibitor in the needed amount in the appropriate solvent or dispersion medium with one or more of the excipients listed above, as required, followed by filtered sterilization. Dispersions can be prepared by incorporating the various sterilized aPKC inhibitor into a sterile vehicle which contains the basic dispersion medium and the required other ingredients from those listed above. Sterile powders for the preparation of sterile injectable solutions can be prepared by vacuum-drying and freeze-drying techniques, which yields a powder of the aPKC inhibitor plus any additional desired ingredient from a previously sterile-filtered solution thereof. The powders can be prepared in such a manner that the particles are porous in nature, which can increase dissolution of the particles. Methods for making porous particles are well known in the art.

Pharmaceutical formulations for parenteral administration can be in the form of a sterile aqueous solution or suspension of particles formed from one or more aPKC inhibitor. Acceptable solvents include, for example, water, Ringer's solution, phosphate buffered saline (PBS), and isotonic sodium chloride solution. The formulation can also be a sterile solution, suspension, or emulsion in a nontoxic, parenterally acceptable diluent or solvent such as 1,3-butanediol.

In some instances, the formulation can be distributed or packaged in a liquid form. In other embodiments, formulations for parenteral administration can be packed as a solid, obtained, for example by lyophilization of a suitable liquid formulation. The solid can be reconstituted with an appropriate carrier or diluent prior to administration.

Solutions, suspensions, or emulsions for parenteral administration can be buffered with an effective amount of buffer necessary to maintain a pH suitable for ocular administration. Suitable buffers include, but are not limited to, acetate, borate, carbonate, citrate, and phosphate buffers.

Solutions, suspensions, or emulsions for parenteral administration can also contain one or more tonicity agents to adjust the isotonic range of the formulation. Suitable tonicity agents include, but are not limited to, glycerin, mannitol, sorbitol, sodium chloride, and other electrolytes.

Solutions, suspensions, or emulsions for parenteral administration can also contain one or more preservatives to prevent bacterial contamination of the ophthalmic preparations. Suitable preservatives include, but are not limited to, polyhexamethylenebiguanidine (PHMB), benzalkonium chloride (BAK), stabilized oxychloro complexes (otherwise known as Purite®), phenylmercuric acetate, chlorobutanol, sorbic acid, chlorhexidine, benzyl alcohol, parabens, thimerosal, and mixtures thereof.

Solutions, suspensions, or emulsions, use of nanotechnology including nanoformulations for parenteral administration can also contain one or more excipients, such as dispersing agents, wetting agents, and suspending agents.

### Topical Formulations

The aPKC inhibitor(s) as described herein can be formulated for topical administration. Suitable dosage forms for topical administration include creams, ointments, salves, sprays, gels, lotions, emulsions, liquids, and transdermal patches. The formulation can be formulated for transmucosal, transepithelial, transendothelial, or transdermal administration. The topical formulations can contain one or more chemical penetration enhancers, membrane permeability agents, membrane transport agents, emollients, surfactants, stabilizers, and combination thereof.

In some embodiments, the aPKC inhibitor can be administered as a liquid formulation, such as a solution or suspension, a semi-solid formulation, such as a lotion or ointment, or a solid formulation. In some embodiments, the aPKC inhibitor can be formulated as liquids, including solutions and suspensions, such as eye drops or as a semi-solid formulation, such as ointment or lotion for topical application to the skin, to the mucosa, such as the eye, to the vagina, or to the rectum.

The formulation can contain one or more excipients, such as emollients, surfactants, emulsifiers, penetration enhancers, and the like.

Suitable emollients include, without limitation, almond oil, castor oil, ceratonia extract, cetostearoyl alcohol, cetyl alcohol, cetyl esters wax, cholesterol, cottonseed oil, cyclomethicone, ethylene glycol palmitostearate, glycerin, glycerin monostearate, glyceryl monooleate, isopropyl myristate, isopropyl palmitate, lanolin, lecithin, light mineral oil, medium-chain triglycerides, mineral oil and lanolin alcohols, petrolatum, petrolatum and lanolin alcohols, soybean oil, starch, stearyl alcohol, sunflower oil, xylitol and combinations thereof. In some embodiments, the emollients can be ethylhexylstearate and ethylhexyl palmitate.

Suitable surfactants include, but are not limited to, emulsifying wax, glyceryl monooleate, polyoxyethylene alkyl ethers, polyoxyethylene castor oil derivatives, polysorbate, sorbitan esters, benzyl alcohol, benzyl benzoate, cyclodextrins, glycerin monostearate, poloxamer, povidone and combinations thereof. In some embodiments, the surfactant can be stearyl alcohol.

Suitable emulsifiers include, but are not limited to, acacia, metallic soaps, certain animal and vegetable oils, and various polar compounds, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxpropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum and combinations thereof. In some embodiments, the emulsifier can be glycerol stearate.

Suitable classes of penetration enhancers include, but are not limited to, fatty alcohols, fatty acid esters, fatty acids, fatty alcohol ethers, amino acids, phospholipids, lecithins, cholate salts, enzymes, amines and amides, complexing agents (liposomes, cyclodextrins, modified celluloses, and diimides), macrocyclics, such as macrocylic lactones, ketones, and anhydrides and cyclic ureas, surfactants, N-methyl pyrrolidones and derivatives thereof, DMSO and related compounds, ionic compounds, azone and related compounds, and solvents, such as alcohols, ketones, amides, polyols (e.g., glycols).

Suitable emulsions include, but are not limited to, oil-in-water and water-in-oil emulsions. Either or both phases of the emulsions can include a surfactant, an emulsifying agent, and/or a liquid non-volatile non-aqueous material. In some embodiments, the surfactant can be a non-ionic surfactant. In other embodiments, the emulsifying agent is an emulsifying wax. In further embodiments, the liquid non-volatile non-aqueous material is a glycol. In some embodiments, the glycol is propylene glycol. The oil phase can contain other suitable oily pharmaceutically acceptable excipients. Suitable oily pharmaceutically acceptable excipients include, but are not limited to, hydroxylated castor oil or sesame oil can be used in the oil phase as surfactants or emulsifiers.

Lotions containing a aPKC inhibitor thereof as described herein are also provided. In some embodiments, the lotion can be in the form of an emulsion having a viscosity of between 100 and 1000 centistokes. The fluidity of lotions can permit rapid and uniform application over a wide surface area. Lotions can be formulated to dry on the skin leaving a thin coat of their medicinal components on the skin's surface.

Creams containing aPKC inhibitor as described herein are also provided. The cream can contain emulsifying agents and/or other stabilizing agents. In some embodiments, the cream is in the form of a cream having a viscosity of greater than 1000 centistokes, typically in the range of 20,000-50,000 centistokes. Creams, as compared to ointments, can be easier to spread and easier to remove.

One difference between a cream and a lotion is the viscosity, which is dependent on the amount/use of various oils and the percentage of water used to prepare the formulations. Creams can be thicker than lotions, can have various uses, and can have more varied oils/butters, depending upon the desired effect upon the skin. In some embodiments of a cream formulation, the water-base percentage can be about 60% to about 75% and the oil-base can be about 20% to about 30% of the total, with the other percentages being the emulsifier agent, preservatives and additives for a total of 100%.

Ointments containing a aPKC inhibitor and a suitable ointment base are also provided. Suitable ointment bases include hydrocarbon bases (e.g., petrolatum, white petrolatum, yellow ointment, and mineral oil); absorption bases (hydrophilic petrolatum, anhydrous lanolin, lanolin, and cold cream); water-removable bases (e.g., hydrophilic ointment), and water-soluble bases (e.g., polyethylene glycol ointments). Pastes typically differ from ointments in that they contain a larger percentage of solids. Pastes are typically more absorptive and less greasy that ointments prepared with the same components.

Also described herein are gels containing a aPKC inhibitor as described herein, a gelling agent, and a liquid vehicle. Suitable gelling agents include, but are not limited to, modified celluloses, such as hydroxypropyl cellulose and hydroxyethyl cellulose; carbopol homopolymers and copolymers; thermoreversible gels and combinations thereof. Suitable solvents in the liquid vehicle include, but are not limited to, diglycol monoethyl ether; alklene glycols, such as propylene glycol; dimethyl isosorbide; alcohols, such as isopropyl alcohol and ethanol. The solvents can be selected for their ability to dissolve the drug. Other additives, which can improve the skin feel and/or emolliency of the formulation, can also be incorporated. Such additives include, but are not limited, isopropyl myristate, ethyl acetate, C₁₂-C₁₅ alkyl benzoates, mineral oil, squalane, cyclomethicone, capric/caprylic triglycerides, and combinations thereof.

Also described herein are foams that can include an aPKC inhibitor as described herein. Foams can be an emulsion in combination with a gaseous propellant. The gaseous propellant can include hydrofluoroalkanes (HFAs). Suitable propellants include HFAs such as 1,1,1,2-tetrafluoroethane (HFA 134a) and 1,1,1,2,3,3,3-heptafluoropropane (HFA 227), but mixtures and admixtures of these and other HFAs that are currently approved or can become approved for medical use are suitable. The propellants can be devoid of hydrocarbon propellant gases, which can produce flammable or explosive vapors during spraying. Furthermore, the foams can contain no volatile alcohols, which can produce flammable or explosive vapors during use.

Buffers can be used to control pH of a composition. The buffers can buffer the composition from a pH of about 4 to a pH of about 7.5, from a pH of about 4 to a pH of about 7, or from a pH of about 5 to a pH of about 7. In some embodiments, the buffer can be triethanolamine.

Preservatives can be included to prevent the growth of fungi and microorganisms. Suitable preservatives include, but are not limited to, benzoic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, and thimerosal.

In certain embodiments, the formulations can be provided via continuous delivery of one or more formulations to a patient in need thereof. For topical applications, repeated application can be done or a patch can be used to provide continuous administration of the noscapine analogs over an extended period of time.

### Enteral Formulations

The aPKC inhibitor or pharmaceutical salt thereof as described herein can be prepared in enteral formulations, such as for oral administration. Suitable oral dosage forms include tablets, capsules, solutions, suspensions, syrups, and lozenges. Tablets can be made using compression or molding techniques well known in the art. Gelatin or non-gelatin capsules can prepared as hard or soft capsule shells, which can encapsulate liquid, solid, and semi-solid fill materials, using techniques well known in the art.

Formulations containing an aPKC inhibitor as described herein can be prepared using pharmaceutically acceptable carriers. As generally used herein “carrier” includes, but is not limited to, diluents, preservatives, binders, lubricants, disintegrators, swelling agents, fillers, stabilizers, and combinations thereof. Polymers used in the dosage form include, but are not limited to, suitable hydrophobic or hydrophilic polymers and suitable pH dependent or independent polymers. Suitable hydrophobic and hydrophilic polymers include, but are not limited to, hydroxypropyl methylcellulose, hydroxypropyl cellulose, hydroxyethyl cellulose, carboxy methylcellulose, polyethylene glycol, ethylcellulose, microcrystalline cellulose, polyvinyl pyrrolidone, polyvinyl alcohol, polyvinyl acetate, and ion exchange resins. “Carrier” also includes all components of the coating composition which can include plasticizers, pigments, colorants, stabilizing agents, and glidants.

Formulations containing an aPKC inhibitor as described herein can be prepared using one or more pharmaceutically acceptable excipients, including diluents, preservatives, binders, lubricants, disintegrators, swelling agents, fillers, stabilizers, and combinations thereof.

Delayed release dosage formulations containing an aPKC inhibitor as described herein can be prepared as described in standard references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, MD, 2000, and “Pharmaceutical dosage forms and drug delivery systems”, 6th Edition, Ansel et al., (Media, PA: Williams and Wilkins, 1995). These references provide information on excipients, materials, equipment and process for preparing tablets and capsules and delayed release dosage forms of tablets, capsules, and granules. These references provide information on carriers, materials, equipment and process for preparing tablets and capsules and delayed release dosage forms of tablets, capsules, and granules.

The formulations containing a aPKC inhibitor as described herein can be coated with a suitable coating material, for example, to delay release once the particles have passed through the acidic environment of the stomach. Suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings can be formed with a different ratio of water soluble polymer, water insoluble polymers and/or pH dependent polymers, with or without water insoluble/water soluble non polymeric excipient, to produce the desired release profile. The coating can be performed on a dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Additionally, the coating material can contain conventional carriers such as plasticizers, pigments, colorants, glidants, stabilization agents, pore formers and surfactants. Optional pharmaceutically acceptable excipients include, but are not limited to, diluents, binders, lubricants, disintegrants, colorants, stabilizers, and surfactants.

Diluents, also referred to as “fillers,” can be used to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar. The usual diluents include inert powdered substances such as starches, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful.

Binders can impart cohesive qualities to a solid dosage formulation, and thus can ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone. Typical tablet binders include substances such as starch, gelatin and sugars such as lactose, fructose, and glucose. Natural and synthetic gums, including acacia, alginates, methylcellulose, and polyvinylpyrrolidone can also be used. Polyethylene glycol, hydrophilic polymers, ethylcellulose and waxes can also serve as binders.

Lubricants can be included to facilitate tablet manufacture. Suitable lubricants include, but are not limited to, magnesium stearate, calcium stearate, stearic acid, glycerol behenate, polyethylene glycol, talc, and mineral oil. A lubricant can be included in a tablet formulation to prevent the tablet and punches from sticking in the die. The lubricant can be chosen from such slippery solids as talc, magnesium and calcium stearate, stearic acid and hydrogenated vegetable oils.

Disintegrants can be used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone® XL from GAF Chemical Corp).

Stabilizers can be used to inhibit or retard drug decomposition reactions which include, by way of example, oxidative reactions. Suitable stabilizers include, but are not limited to, antioxidants, butylated hydroxytoluene (BHT); ascorbic acid, its salts and esters; Vitamin E, tocopherol and its salts; sulfites such as sodium metabisulphite; cysteine and its derivatives; citric acid; propyl gallate, and butylated hydroxyanisole (BHA).

### Additional Active Agents

In some embodiments, an amount of one or more additional active agents are included in the pharmaceutical formulation containing an aPKC inhibitor or pharmaceutical salt thereof. Suitable additional active agents include, but are not limited to, DNA, RNA, amino acids, peptides, polypeptides, antibodies, aptamers, ribozymes, guide sequences for ribozymes that inhibit translation or transcription of essential tumor proteins and genes, hormones, immunomodulators, antipyretics, anxiolytics, antipsychotics, analgesics, antispasmodics, anti-inflammatories, anti-histamines, anti-infectives, and chemotherapeutics. Other suitable additional active agents include, but are not limited to, statins, cholesterol lowering drugs, glucose lowering drugs. The aPKC inhibitor(s) can be used as a monotherapy or in combination with other active agents for treatment of metabolic disorder (diabetes, high-cholesterol, hyperlipidemia, high-triglycerides).

Suitable hormones include, but are not limited to, amino-acid derived hormones (e.g. melatonin and thyroxine), small peptide hormones and protein hormones (e.g. thyrotropin-releasing hormone, vasopressin, insulin, growth hormone, luteinizing hormone, follicle-stimulating hormone, and thyroid-stimulating hormone), eiconsanoids (e.g. arachidonic acid, lipoxins, and prostaglandins), and steroid hormones (e.g. estradiol, testosterone, tetrahydro testosteron cortisol).

Suitable immunomodulators include, but are not limited to, prednisone, azathioprine, 6-MP, cyclosporine, tacrolimus, methotrexate, interleukins (e.g. IL-2, IL-7, and IL-12), cytokines (e.g. interferons (e.g. IFN-α, IFN-β, IFN-ε, IFN-κ, IFN-ω, and IFN-γ), granulocyte colony-stimulating factor, and imiquimod), chemokines (e.g. CCL3, CCL26 and CXCL7), cytosine phosphate-guanosine, oligodeoxynucleotides, glucans, antibodies, and aptamers).

Suitable antipyretics include, but are not limited to, non-steroidal anti-inflammants (e.g. ibuprofen, naproxen, ketoprofen, and nimesulide), aspirin and related salicylates (e.g. choline salicylate, magnesium salicylae, and sodium salicaylate), paracetamol/acetaminophen, metamizole, nabumetone, phenazone, and quinine.

Suitable anxiolytics include, but are not limited to, benzodiazepines (e.g. alprazolam, bromazepam, chlordiazepoxide, clonazepam, clorazepate, diazepam, flurazepam, lorazepam, oxazepam, temazepam, triazolam, and tofisopam), serotenergic antidepressants (e.g. selective serotonin reuptake inhibitors, tricyclic antidepresents, and monoamine oxidase inhibitors), mebicar, afobazole, selank, bromantane, emoxypine, azapirones, barbituates, hyxdroxyzine, pregabalin, validol, and beta blockers.

Suitable antipsychotics include, but are not limited to, benperidol, bromoperidol, droperidol, haloperidol, moperone, pipaperone, timiperone, fluspirilene, penfluridol, pimozide, acepromazine, chlorpromazine, cyamemazine, dizyrazine, fluphenazine, levomepromazine, mesoridazine, perazine, pericyazine, perphenazine, pipotiazine, prochlorperazine, promazine, promethazine, prothipendyl, thioproperazine, thioridazine, trifluoperazine, triflupromazine, chlorprothixene, clopenthixol, flupentixol, tiotixene, zuclopenthixol, clotiapine, loxapine, prothipendyl, carpipramine, clocapramine, molindone, mosapramine, sulpiride, veralipride, amisulpride, amoxapine, aripiprazole, asenapine, clozapine, blonanserin, iloperidone, lurasidone, melperone, nemonapride, olanzaprine, paliperidone, perospirone, quetiapine, remoxipride, risperidone, sertindole, trimipramine, ziprasidone, zotepine, alstonie, befeprunox, bitopertin, brexpiprazole, cannabidiol, cariprazine, pimavanserin, pomaglumetad methionil, vabicaserin, xanomeline, and zicronapine.

Suitable analgesics include, but are not limited to, paracetamol/acetaminophen, non-steroidal anti-inflammants (e.g. ibuprofen, naproxen, ketoprofen, and nimesulide), COX-2 inhibitors (e.g. rofecoxib, celecoxib, and etoricoxib), opioids (e.g. morphine, codeine, oxycodone, hydrocodone, dihydromorphine, pethidine, buprenorphine), tramadol, norepinephrine, flupiretine, nefopam, orphenadrine, pregabalin, gabapentin, cyclobenzaprine, scopolamine, methadone, ketobemidone, piritramide, and aspirin and related salicylates (e.g. choline salicylate, magnesium salicylae, and sodium salicaylate).

Suitable antispasmodics include, but are not limited to, mebeverine, papverine, cyclobenzaprine, carisoprodol, orphenadrine, tizanidine, metaxalone, methodcarbamol, chlorzoxazone, baclofen, dantrolene, baclofen, tizanidine, and dantrolene.

Suitable anti-inflammatories include, but are not limited to, prednisone, non-steroidal anti-inflammants (e.g. ibuprofen, naproxen, ketoprofen, and nimesulide), COX-2 inhibitors (e.g. rofecoxib, celecoxib, and etoricoxib), and immune selective anti-inflammatory derivatives (e.g. submandibular gland peptide-T and its derivatives).

Suitable anti-histamines include, but are not limited to, H₁-receptor antagonists (e.g. acrivastine, azelastine, bilastine, brompheniramine, buclizine, bromodiphenhydramine, carbinoxamine, cetirizine, chlorpromazine, cyclizine, chlorpheniramine, clemastine, cyproheptadine, desloratadine, dexbromapheniramine, dexchlorpheniramine, dimenhydrinate, dimetindene, diphenhydramine, doxylamine, ebasine, embramine, fexofenadine, hydroxyzine, levocetirzine, loratadine, meclozine, mirtazapine, olopatadine, orphenadrine, phenindamine, pheniramine, phenyltoloxamine, promethazine, pyrilamine, quetiapine, rupatadine, tripelennamine, and triprolidine), H₂-receptor antagonists (e.g. cimetidine, famotidine, lafutidine, nizatidine, rafitidine, and roxatidine), tritoqualine, catechin, cromoglicate, nedocromil, and β2-adrenergic agonists.

Suitable anti-infectives include, but are not limited to, amebicides (e.g. nitazoxanide, paromomycin, metronidazole, tnidazole, chloroquine, and iodoquinol), aminoglycosides (e.g. paromomycin, tobramycin, gentamicin, amikacin, kanamycin, and neomycin), anthelmintics (e.g. pyrantel, mebendazole, ivermectin, praziquantel, abendazole, miltefosine, thiabendazole, oxamniquine), antifungals (e.g. azole antifungals (e.g. itraconazole, fluconazole, posaconazole, ketoconazole, clotrimazole, miconazole, and voriconazole), echinocandins (e.g. caspofungin, anidulafungin, and micafungin), griseofulvin, terbinafine, flucytosine, and polyenes (e.g. nystatin, and amphotericin b), antimalarial agents (e.g. pyrimethamine/sulfadoxine, artemether/lumefantrine, atovaquone/proquanil, quinine, hydroxychloroquine, mefloquine, chloroquine, doxycycline, pyrimethamine, and halofantrine), antituberculosis agents (e.g. aminosalicylates (e.g. aminosalicylic acid), isoniazid/rifampin, isoniazid/pyrazinamide/rifampin, bedaquiline, isoniazid, ethanmbutol, rifampin, rifabutin, rifapentine, capreomycin, and cycloserine), antivirals (e.g. amantadine, rimantadine, abacavir/lamivudine, emtricitabine/tenofovir, cobicistat/elvitegravir/emtricitabine/tenofovir, efavirenz/emtricitabine/tenofovir, avacavir/lamivudine/zidovudine, lamivudine/zidovudine, emtricitabine/tenofovir, emtricitabine/opinavir/ritonavir/tenofovir, interferon alfa-2v/ribavirin, peginterferon alfa-2b, maraviroc, raltegravir, dolutegravir, enfuvirtide, foscamet, fomivirsen, oseltamivir, zanamivir, nevirapine, efavirenz, etravirine, rilpiviirine, delaviridine, nevirapine, entecavir, lamivudine, adefovir, sofosbuvir, didanosine, tenofovir, avacivr, zidovudine, stavudine, emtricitabine, xalcitabine, telbivudine, simeprevir, boceprevir, telaprevir, lopinavir/ritonavir, fosamprenvir, dranuavir, ritonavir, tipranavir, atazanavir, nelfinavir, amprenavir, indinavir, sawuinavir, ribavirin, valcyclovir, acyclovir, famciclovir, ganciclovir, and valganciclovir), carbapenems (e.g. doripenem, meropenem, ertapenem, and cilastatin/imipenem), cephalosporins (e.g. cefadroxil, cephradine, cefazolin, cephalexin, cefepime, ceflaroline, loracarbef, cefotetan, cefuroxime, cefprozil, loracarbef, cefoxitin, cefaclor, ceftibuten, ceftriaxone, cefotaxime, cefpodoxime, cefdinir, cefixime, cefditoren, cefizoxime, and ceftazidime), glycopeptide antibiotics (e.g. vancomycin, dalbavancin, oritavancin, and telvancin), glycylcyclines (e.g. tigecycline), leprostatics (e.g. clofazimine and thalidomide), lincomycin and derivatives thereof (e.g. clindamycin and lincomycin), macrolides and derivatives thereof (e.g. telithromycin, fidaxomicin, erthromycin, azithromycin, clarithromycin, dirithromycin, and troleandomycin), linezolid, sulfamethoxazole/trimethoprim, rifaximin, chloramphenicol, fosfomycin, metronidazole, aztreonam, bacitracin, beta lactam antibiotics (benzathine penicillin (benzatihine and benzylpenicillin), phenoxymethylpenicillin, cloxacillin, flucoxacillin, methicillin, temocillin, mecillinam, azlocillin, mezlocillin, piperacillin, amoxicillin, ampicillin, bacampicillin, carbenicillin, piperacillin, ticarcillin, amoxicillin/clavulanate, ampicillin/sulbactam, piperacillin/tazobactam, clavulanate/ticarcillin, penicillin, procaine penicillin, oxacillin, dicloxacillin, nafcillin, cefazolin, cephalexin, cephalosporin C, cephalothin, cefaclor, cefamandole, cefuroxime, cefotetan, cefoxitin, cefiximine, cefotaxime, cefpodoxime, ceftazidime, ceftriaxone, cefepime, cefpirome, ceftaroline, biapenem, doripenem, ertapenem, faropenem, imipenem, meropenem, panipenem, razupenem, tebipenem, thienamycin, azrewonam, tigemonam, nocardicin A, taboxinine, and beta-lactam), quinolones (e.g. lomefloxacin, norfloxacin, ofloxacin, qatifloxacin, moxifloxacin, ciprofloxacin, levofloxacin, gemifloxacin, moxifloxacin, cinoxacin, nalidixic acid, enoxacin, grepafloxacin, gatifloxacin, trovafloxacin, and sparfloxacin), sulfonamides (e.g. sulfamethoxazole/trimethoprim, sulfasalazine, and sulfasoxazole), tetracyclines (e.g. doxycycline, demeclocycline, minocycline, doxycycline/salicyclic acid, doxycycline/omega-3 polyunsaturated fatty acids, and tetracycline), and urinary anti-infectives (e.g. nitrofurantoin, methenamine, fosfomycin, cinoxacin, nalidixic acid, trimethoprim, and methylene blue).

Suitable chemotherapeutics include, but are not limited to, paclitaxel, brentuximab vedotin, doxorubicin, 5-FU (fluorouracil), everolimus, pemetrexed, melphalan, pamidronate, anastrozole, exemestane, nelarabine, ofatumumab, bevacizumab, belinostat, tositumomab, carmustine, bleomycin, bosutinib, busulfan, alemtuzumab, irinotecan, vandetanib, bicalutamide, lomustine, daunorubicin, clofarabine, cabozantinib, dactinomycin, ramucirumab, cytarabine, cytoxan, cyclophosphamide, decitabine, dexamethasone, docetaxel, hydroxyurea, decarbazine, leuprolide, epirubicin, oxaliplatin, asparaginase, estramustine, cetuximab, vismodegib, aspargainase erwinia chyrsanthemi, amifostine, etoposide, flutamide, toremifene, fulvestrant, letrozole, degarelix, pralatrexate, methotrexate, floxuridine, obinutuzumab, gemcitabine, afatinib, imatinib mesylatem, carmustine, eribulin, trastuzumab, altretamine, topotecan, ponatinib, idarubicin, ifosfamide, ibrutinib, axitinib, interferon alfa-2a, gefitinib, romidepsin, ixabepilone, ruxolitinib, cabazitaxel, ado-trastuzumab emtansine, carfilzomib, chlorambucil, sargramostim, cladribine, mitotane, vincristine, procarbazine, megestrol, trametinib, mesna, strontium-89 chloride, mechlorethamine, mitomycin, busulfan, gemtuzumab ozogamicin, vinorelbine, filgrastim, pegfilgrastim, sorafenib, nilutamide, pentostatin, tamoxifen, mitoxantrone, pegaspargase, denileukin diftitox, alitretinoin, carboplatin, pertuzumab, cisplatin, pomalidomide, prednisone, aldesleukin, mercaptopurine, zoledronic acid, lenalidomide, rituximab, octretide, dasatinib, regorafenib, histrelin, sunitinib, siltuximab, omacetaxine, thioguanine (tioguanine), dabrafenib, erlotinib, bexarotene, temozolomide, thiotepa, thalidomide, BCG, temsirolimus, bendamustine hydrochloride, triptorelin, aresnic trioxide, lapatinib, valrubicin, panitumumab, vinblastine, bortezomib, tretinoin, azacitidine, pazopanib, teniposide, leucovorin, crizotinib, capecitabine, enzalutamide, ipilimumab, goserelin, vorinostat, idelalisib, ceritinib, abiraterone, epothilone, tafluposide, azathioprine, doxifluridine, vindesine, and all-trans retinoic acid.

Methods of Using the aPKC Inhibitor(s) and Formulations Thereof

Hyperinsulinemia in the periphery can contribute to insulin resistance and ultimately many disorders such as diabetes and metabolic syndrome. In the liver aPKC activity can be increased in response to hyperinsulinemia, which can stimulate the production of lipids and other insulin resistant factors that can reduce glucose uptake by muscle cells, which increases blood glucose concentration and further stimulates insulin production. Additionally, increased aPKC in the liver can block Akt, which can increase glucose production by the liver, which can increase blood glucose concentration and further stimulates insulin production. In the muscle hyperinsulinemia decreases the expression and/or activity of glucose transporters, which decreases glucose uptake from the blood stream by muscle cells. This increases blood glucose concentration, which can stimulate the production of insulin from islet β-cells and further contribute to hyperinsulinemia.

Previously, it was thought that the brain can become insulin resistant. However, as demonstrated elsewhere herein, the brain is insulin responsive but hyperinsulinemia can result in dysregulation of various signaling pathways, which can be the result of increased aPKC expression in the CNS. Hyperinsulinemia in the brain can result in increased activity of aPKC isoform, PKC-λ/ι and Akt via stimulation of the IRS 1/2, PI3K pathway. Hyperactivity of these pathways increases production of BACE1, Aβ_{1-40/42}, and p-Tau and decreased PGC-1α, which can directly contribute to the development of neurodegenerative disorders, such as AD. In short, hyperinsulinemia in the CNS can result in aberrant signaling, which can be restored to substantially normal by reduction of hyperinsulinemia or direct inhibition of aPKC in the CNS.

The aPKC inhibitors that can cross the blood brain barrier can directly inhibit aPKC in the brain and, inter alia, decrease BACE1, Aβ_{1-40/42}, and p-Tau and increase PGC-1α expression and/or activity, thereby treating and/or preventing a neurodegenerative disorder or a symptom thereof. The aPKC inhibitors that do not cross the blood brain barrier can reduce systemic hyperinsulinemia by their action in the liver and/or muscle. The reduction in hyperinsulinemia reduces stimulation of aPKC and/or Akt in the CNS, which can work to restore normal signaling in the CNS.

The aPKC inhibitors and formulations thereof described herein can be administered to a subject in need thereof. The subject in need thereof can be hyperinsulinemic in the periphery and/or CNS, have diabetes (type 1 or type 2), metabolic syndrome, obesity, a neurodegenerative disorder (e.g. AD) a symptom thereof, or a complication thereof (e.g. high blood sugar and/or cardiovascular disorders). The subject in need thereof can be symptomatic or asymptomatic. The subject in need thereof can currently be taking insulin to treat metabolic disorders as described above. In some aspects, the subject in need thereof does not have diabetes, but has a symptomatic or asymptomatic neurodegenerative disorder, such as, but not limited to, AD.

The amount of the aPKC inhibitors and formulations thereof delivered to the subject in need thereof can be an amount effective to reduce blood glucose level, reduce hyperinsulinemia in the periphery and/or CNS, decrease phosphorylation an/or activity of Akt, such as in the CNS, directly and/or indirectly decrease activity of aPKC in the CNS, decrease activity of β-secretase in the CNS, decrease the amount of Aβ_{1-40/42} in the CNS, decrease the amount of thr-231-phospho-tau in the CNS, decrease the activity of aPKC isoform PKC-λ/ι in the CNS, decrease p-FoxOs in the CNS, decrease pGSK3β in the CNS, decrease p-mTOR in the CNS, increase the activity of FoxO1, FoxO3a, or FoxO1 and FoxO3a, and/or increase PGC-1α in the CNS. The amount of the aPKC inhibitors and formulations thereof administered to the subject can treat or prevent a neurodegenerative disorder or a symptom thereof. Such neurodegenerative disorders can be, without limitation, AD.

It will be appreciated that “co-administered” or “co-administration” can refer to an additional compound that is included in the formulation or provided in a dosage form separate from the aPKC inhibitor or formulation thereof. The effective amount of aPKC inhibitor or formulation thereof, such as those described herein, can range from about 0.1, 1, 5, 10, 20, 30, 40, 60, 80, 90, 100, 150, 200, 250, 300, 350, 400, or 450 mg/kg to about 5000 mg/kg. In some embodiments, the effective amount ranges from about 0.1, 1, 5, 10, 15, 20, 25, 30, 35, 40, 45, 50, 55, 60, 65 mg/kg to 70 mg/kg. In additional embodiments, the effective amount of the aPKC inhibitor or formulation thereof can range from about 100 mg/kg. If further embodiments, the effective amount can range from about 0.1 mg to about 1000 mg. In some embodiments, the effective amount can range from about 500 mg to about 1000 mg. The effective amount in a can range from about 0.1, 1, 5, 10, 20, 30, 40, 50, 60, 70, 80, 90, 100, 150, 200, 250, 300, 350, 400, 450, or 500 mg/kg to about 5000 mg/kg per dose or per day.

Administration of the aPKC inhibitor and/or formulations thereof can be systemic and/or localized. The aPKC inhibitors and formulations thereof described herein can be administered to the subject in need thereof one or more times per day. The aPKC inhibitors and formulations thereof can be administered one or more times per day every other day. In an embodiment, the compound(s) and/or formulation(s) thereof can be administered once daily. The aPKC inhibitors and formulations thereof and/or formulation(s) thereof can be administered given once daily for 1, 2, 3, 4, 5, 6, 7, 8, 9, 10, 11, 12, 13, 14, or more consecutive days. In some embodiments, when administered, an effective amount of the compounds and/or formulations are administered to the subject in need thereof. The compound(s) and/or formulation(s) thereof can be administered one or more times per week. In some embodiments the compound(s) and/or formulation(s) thereof can be administered 1 day per week. In other embodiments, the compound(s) and/or formulation(s) thereof can be administered 2 to 7 days per week.

In some embodiments, the aPKC inhibitor(s) and/or formulation(s) thereof, can be administered in a dosage form. The amount or effective amount of the compound(s) and/or formulation(s) thereof can be divided into multiple dosage forms. For example, the effective amount can be split into two dosage forms and the one dosage forms can be administered, for example, in the morning, and the second dosage form can be administered in the evening.

Although the effective amount is given over two doses, in one day, the subject receives the effective amount. In some embodiments the effective amount is about 0.1 to about 5000 mg per day. The effective amount in a dosage form can range from about 0.1, 1, 5, 10, 20, 30, 40, 50, 60, 70, 80, 90, 100, 150, 200, 250, 300, 350, 400, 450, or 500 mg/kg to about 5000 mg/kg per dose or per day. The dosage form can be formulated for oral, nasal, vaginal, intravenous, transdermal, subcutaneous, intraperitoneal, or intramuscular administration. Preparation of dosage forms for various administration routes are described elsewhere herein.

### REFERENCES

Bruckle, W.; Dexel, T.; Grasedyck, K.; Schattenkirchner, M. Treatment of psoriatic arthritis with auranofin and gold sodium thiomalate. Clin. Rheumatol. 1994, 13, 209-216
Butler, A. M.; Scotti Buzhardt, M. L.; Erdogan, E.; Li, S.; Inman, K. S.; Fields, A. P.; Murray, N. R. A small molecule inhibitor of atypical protein kinase C signaling inhibits pancreatic cancer cell transformed growth and invasion. Oncotarget 2015, 6, 15297-15310.
Erdogan, E.; Lamark, T.; Stallings-Mann, M.; Lee, J.; Pellecchia, M.; Thompson, E. A.; Johansen, T.; Fields, A. P. Aurothiomalate inhibits transformed growth by targeting the PB1 domain of protein kinase Ct. J. Biol. Chem. 2006, 281, 28450-28459.
Jatoi, A.; Radecki Breitkopf, C.; Foster, N. R.; Block, M. S.; Grudem, M.; Wahner Hendrickson, A.; Carlson, R. E.; Barrette, B.; Karlin, N.; Fields, A. P. A mixed-methods feasibility trial of protein kinase Ct inhibition with auranofin in asymptomatic ovarian cancer patients. Oncology 2015, 88, 208-213.
Jessop, J. D.; O'Sullivan, M. M.; Lewis, P. A.; Williams, L. A.; Camilleri, J. P.; Plant, M. J.; Coles, E. C. A long-term five-year randomized controlled trial of hydroxychloroquine, sodium aurothiomalate, auranofin and penicillamine in the treatment of patients with rheumatoid arthritis. Br. J Rheumatol. 1998, 37, 992-1002.
Kambhampati, S. Phase I and II Study of Auranofin in Chronic Lymphocytic Leukemia. 2016. Li, H.; Hu., J.; Wu, S.; Wang, L.; Cao, X.; Zhang, X.; Dai, B.; Cao, M.; Shao, R.; Zhang, R.; et al. Auranofin-mediated inhibition of PI3K/AKT/mTOR axis and anticancer activity in non-small cell lung cancer cells. Oncotarget 2016, 7, 3548.
Mirabelli, C. K.; Johnson, R. K.; Hill, D. T.; Faucette, L. F.; Girard, G. R.; Kuo, G. Y.; Sung, C. M.; Crooke, S. T. Correlation of the in vitro cytotoxic and in vivo antitumor activities of gold(I) coordination complexes. J. Med. Chem. 1986, 29, 218-223.
Regala, R. P.; Thompson, E. A.; Fields, A. P. Atypical protein kinase Ct expression and aurothiomalate sensitivity in human lung cancer cells. Cancer Res. 2008, 68, 5888-5895.

### EXAMPLES

### Example 1: Control of β-Site Amyloid Precursor Protein-Cleaving Enzyme-1 Expression by Protein Kinase C-λ/ι and Nuclear Factor κ-B

### SUMMARY

Background: β-Amyloid precursor protein-cleaving enzyme-1 (BACE1) initiates the production of Aβ-peptides that form AD-plaque in Alzheimer's disease.

Methods: Reportedly, acute insulin treatment in normal mice, and hyperinsulinemia in high-fat-fed (HFF) obese/diabetic mice, increase BACE1 activity and levels of Aβ-peptides and phospho-thr-231-tau in the brain; moreover, these effects are blocked by PKC-λ/ι inhibitors. However, as chemical inhibitors may affect unsuspected targets, we presently used knockout methodology to further examine PKC-λ/ι requirements. We found that total-body heterozygous PKC-λ knockout reduced acute stimulatory effects of insulin and chronic effects of hyperinsulinemia in HFF/obese/diabetic mice, on brain PKC-λ activity and production of Aβ1-40/42 and phospho-thr-231-tau. This protection in HFF mice may reflect that hepatic PKC-λ haploinsufficiency prevents the development of glucose intolerance and hyperinsulinemia.

Results: Heterozygous knockout of PKC-λ exhibited markedly reduced brain levels of BACE1 protein and mRNA, and this may reflect diminished activation of nuclear factor kappa-B (NFκB), which is activated by PKC-λ and increases BACE1 and proinflammatory cytokine transcription. Intravenous administration of aPKC inhibitor diminished aPKC activity and BACE1 levels by 50% in the brain and 90% in the liver, nasally-administered inhibitor reduced aPKC activity and BACE1 mRNA and protein levels by 50-70% in the brain while sparing the liver. Additionally, 24-hour insulin treatment in cultured human-derived neurons increased NFκB activity and BACE1 levels, and these effects were blocked by PKC-λ/ι inhibitors.

Conclusion: PKC-λ/ι controls NFκB activity and BACE1 expression; PKC-λ/ι inhibitors may be used nasally to target brain PKC-λ/ι or systemically to block both liver and brain PKC-λ/ι, to regulate NFκB-dependent BACE1 and proinflammatory cytokine expression.

### Introduction

Alzheimer's disease (AD) is characterized by increases in brain levels of potentially neurotoxic Aβ-peptides (Aβ) that aggregate to form pathological plaques with associated increases in neurofibrillary tangles of phospho-tau. Increases in Aβ-peptides are initiated by aspartyl-peptidase action of neuronal β-secretase [β-site amyloid precursor protein (APP)-cleaving enzyme-1 (BACE1)] on βAPP, followed by γsecretase action [1, 2].

Some [3], but not all, studies have observed increases in brain levels and/or activity of BACE1 in sporadic non-familial late-onset AD. However, Aβ production is complex and may also involve alterations in trafficking and interactions between BACE1-containing and βAPP-containing vesicles [4], and Aβ levels can be altered by changes in AR clearance and actions of α-secretase, which cleaves βAPP within the Aβ sequence, thus precluding the production of full-length Aβ-peptides. Irrespective of whether BACE1 levels are altered in various forms of AD, BACE1 action is critical for producing increases in Aβ, and possibly phospho-tau. Regardless of how increases in AR and phospho-tau are produced, these pathologic aberrations are thought to cause impairments in hippocampal-dependent learning and memory functions eventually leading to neurodegenerative pathological processes throughout the brain.

AD-related elevations in brain BACE1 activity or levels have been attributed to a variety of factors [1, 2], perhaps most notably, those involving oxidative and inflammatory stress [5]. In this regard, it appears that BACE1 expression in the brain and perhaps other tissues is mainly controlled by the master regulator of inflammation, nuclear factor kappa-B (NFκB), which also controls the expression of proinflammatory cytokines, e.g., tumor necrosis factor-α (TNF-α), interleukin 1-β (IL-1β), and IL-6 [6-8]. This inter-relationship between BACE1 and proinflammatory cytokines raises the possibility that various AD risk factors may converge at a single definable signaling pathway that leads to NFκB activation and development of AD pathology due to increases in levels of BACE1 and proinflammatory cytokines.

One apparent and widespread risk factor for AD development is insulin-resistant forms of obesity, the associated metabolic syndrome, and type 2 diabetes (T2D). Indeed, AD commonly co-exists with obesity/T2D: e.g., 80% of AD patients seen at the Mayo Clinic had overt T2D or fasting glucose intolerance [9], a common feature of obesity and the metabolic syndrome that, untreated, commonly progresses to T2D. As systemic insulin resistance antedates AD in obesity/T2D-associated AD, and, as brain insulin receptor (IR) activity is partly diminished in late-onset, “non-diabetic” AD [10], it is postulated that the brain itself is insulin-resistant, and this resistance, with consequent hypo-insulinization of the brain, abets development of both diabetes-associated and diabetes-unassociated AD [11]. Whereas this mechanism of diminished insulin action in the brain may obtain in AD in conditions of normal or low (as in later stages of T2D) plasma insulin levels, or when AD brain damage impairs insulin action more fully at the level of the IR, or its immediate substrate IRS-1/2) or beyond, a different scenario apparently exists in hyperinsulinemic conditions at earlier stages of AD development, when brain IRs are sufficiently activated in response to elevated plasma insulin levels.

Indeed, we found that hyperinsulinemia in multiple, etiologically-diverse forms of obesity/T2D in mice and monkeys (including widely used high-fat-fed (HFF) and ob/ob mouse models (provokes maximal increases in activities of brain IR and phosphatidylinositol-3-kinase (PI3K)-dependent signaling factors that are activated by PI3K-induced increases in phosphatidylinositol-3,4,5-(PO4)3 (PIP 3) and mediate most insulin effects, viz., Akt and atypical PKC (aPKC) [12,13]. Moreover, chemical inhibitor studies show that aPKC, rather than Akt, increases BACE1 activity and levels of Aβ1-40/42 and phospho-thr-231-tau in both hyperinsulinemic mice and during acute insulin action in brains of normal mice, mouse hippocampal slices, and cultured human-derived neurons [13]. Very importantly, in an obese/T2D mouse model, a simple correction of hyperinsulinemia by reduction of hepatic gluconeogenic enzymes through use of a chemical inhibitor (aurothiomalate) that selectively blocks liver, but not the brain, aPKC, simultaneously corrects elevations in brain Akt and aPKC and aPKC-dependent increases in BACE1 activity and levels of Aβ1-40/42 and phospho-thr-231-tau [12].

We also found [13] that a similar abrogation of increases in brain BACE1 activity and levels of Aβ1-40/42 and phospho-thr-231-tau seen in HFF mice is achieved by other chemical inhibitors of PKC-λ/ι that cross the blood brain barrier (BB-B) and directly inhibit the insulin-sensitive aPKC, i.e., 70 kDa PKC-λ/ι in the brain, without inhibiting brain Akt or the putative long term memory protein, 50 kDa PKMζ, a truncated form of PKC-ζ that contains the catalytic domain, but, lacking a regulatory domain and its auto-inhibitory pseudosubstrate sequence, is constitutively-active.

However, as chemical inhibitors may have non-specific effects that are mediated by unsuspected targets, we presently used gene-knockout methodology to further examine PKC-λ/ι requirements for insulin-dependent alterations in BACE1, Aβ1-40/42, and phospho-thr-231-tau. Whereas, homozygous PKC-λ knockout is embryonic lethal, mice heterozygous for PKC-λ are seemingly normal, but nevertheless have proved to be very useful for revealing PKC-λ/ι requirements in specific tissues. Thus, as total-body heterozygous PKC-λ-knockout (TB/HETλKO) mice are deficient in hepatic PKC-λ, and, as hepatic PKC-λ/ι restrains basal and insulin-regulated Akt-mediated phosphorylation (and thus inhibition) of hepatic FoxO1 and PGC-1α [14-16], Akt phosphorylation is constitutive in TB/HETλKO mice; and these TB/HETλKO mice, therefore, have diminished levels of both lipogenic and gluconeogenic enzymes that, when excessive, produce clinical features of obesity, the metabolic syndrome and T2D [14]. Accordingly, TB/HETλKO mice are protected from developing increases in plasm lipid levels, gluconeogenesis, glucose intolerance, and hyperinsulinemia in response to a high-fat diet (HFD) [14].

Here, we questioned whether: (a) haploinsufficiency of hepatic PKC-λ in TB/HETλKO mice and failure to develop hyperinsulinemia during high fat-feeding protects the brain against HFD/hyperinsulinemia-induced hyperactivation of both Akt and aPKC in the brain, and aPKC-dependent increases in brain BACE1, Aβ1-40/42 and phospho-thr-231-tau; (b) haplo-insufficiency of brain PKC-λ reduces acute insulin-induced increases in brain aPKC activity, and aPKC-dependent changes in BACE1, Aβ1-40/42 and phospho-thr-231-tau; and (c) haplo-insufficiency of brain PKC-λ impairs learning/memory functions, a possibility arising from findings following acute knockdown of hippocampal PKC-λ [17], but not, or much less, following chronic knockout of hippocampal PKC-λ, in which PKMζ or other PKCs compensate for losses of PKC-λ/ι [18].

However, we surprisingly found that haploinsufficiency of brain PKC-λ in TB/HETλKO was attended by marked decreases in brain BACE1 protein and mRNA levels. Given that BACE1 expression in the brain is reported to be largely dependent on NFκB [6-8], we questioned whether brain NFκB activity and NFκB-dependent expression of proinflammatory cytokines are increased in hyperinsulinemic HFF mice and decreased by PKC-λ/ι haploinsufficiency.

Additionally, we questioned whether: (a) nasal administration of chemical inhibitors can be used to specifically target brain PKC-λ/ι and thereby diminish brain BACE1 levels, and (b) intravenous administration of chemical inhibitors can be used to simultaneously target liver and brain PKC-λ/ι and thereby alter BACE1 levels in both tissues.

### Materials and Methods

### Mouse Studies

Mice (C57Bl/6 and 129P2/SV backgrounds) were housed in environmentally-controlled rooms and, as previously described [14], fed either a regular diet of high-carbohydrate/low-fat mouse chow supplying 10% of calories from fat or, where indicated, a hypercaloric Western-style, high-fat diet (HFD) supplying 40% of calories from fat (Harlan Industries, Madison, Wisconsin). Note that, in WT mice, this hypercaloric diet, like a commonly-used 60% HFD, regularly induces an insulin-resistant form of obesity/T2D, owing to increased activation of hepatic aPKC by diet-dependent increases in ceramide, followed by impairment of insulin-stimulated Akt action and activation, followed by increases in activity of FoxO1 and PGC-1α, followed by increased expression of hepatic gluconeogenic and lipogenic enzymes [14-16].

As described [14], brains were harvested from 5-month-old male wild-type (WT) and littermate total-body heterozygous PKC-λ knockout (TB/HEtλKO) mice, generated by mating male and female TB/HETλKO mice, to produce WT and TB/HETλKO mice at a 1:2 ratio, along with homozygous KO offspring that die in utero. These WT and TB/HETλKO mice were fed either a regular mouse chow diet or the above-described Western HFD over 10 weeks in studies described previously [14], snap-frozen in liquid N2, and stored at −80° C. until present usage. Results showing HFD-induced increases in serum levels of glucose and insulin levels during intraperitoneal (IP) glucose tolerance testing in WT mice, and normal serum levels of glucose and insulin levels during IP glucose tolerance testing in TB/HETλKO mice, were reported previously [14].

Where indicated, mice were treated IP with vehicle or a maximally-effective dose of insulin (1 U/kg body weight) or vehicle (controls) 15-min before euthanization by administration of Xylazine/Ketamine or CO2, followed by whole body perfusion with phosphate-buffered saline (PBS) and rapid removal of liver, brain, and other tissues, as described [12, 13].

Where indicated, to selectively inhibit brain aPKC, mice were treated once daily by nasal “sniffing”, i.e., by dropping 10 μL of vehicle or vehicle containing PKC-λ/ι inhibitor, ICAP, (total daily dose of 100 mg/kg body weight) into each nostril while holding the mouse in a supine position.

As described [14, 15], all experimental procedures involving mice were approved by the Institutional Animal Care and Use Committees of the University of South Florida and the James A. Haley Veterans Administration Research and Development Committee.

### Studies in Cultures of LA1-5s Human Neuroblastoma-Derived Neuronal Cells.

LA1-5s neuronal cells were originally obtained from non-catecholamine-producing neuroblastoma of a 3-year-old female and are commercially available from the European Collection of Authenticated Cell Cultures. Incubation conditions were as described [13].

### PKC-λ/ι Inhibitors

1H-imidazole-4-carboxamide,-5-amino-1-[2,3-dihydroxy-4-[(hydroxyl)methyl]cyclopentyl-[1R-(1a,2b,3b,4a)] (ICAP), was custom-synthesized by USF Chemistry Department members and purified to >95%, as per NMR and LC/MS analyses. Note that inactive ICAP is converted intracellularly by adenosine kinase to the phosphorylated active compound, [1 H-imidazole-4-carboxamide,5-amino]-[2,3-dihydroxy-4-[(phosphono-oxy) methyl]cyclopentane-[1R-(1a, 2b, 3b, 4a)] (ICAPP) [19]. Also note: (a) ICAPP potently [IC50, 1-10 nM] inhibits recombinant PKC-ι/λ, but has no effect on recombinant forms of PKC-α, PKC-β, PKC-δ, PKCε, or PKC-θ, and only weakly [(IC50, 200-500 nM] inhibits recombinant PKC-ζ [15, 19, 20]; (b) in isolated human hepatocytes, ICAPP and ICAP inhibit aPKC but not Akt2 or AMP-activated kinase (AMPK), and moreover, increase FoxO1 phosphorylation/inactivation by inhibiting aPKC, and thereby diminishing, gluconeogenic enzyme expression [16, 20]; (c) in intact mice, employed doses of ICAPP and ICAP inhibit aPKC in liver, but not in muscle or adipose tissue [15]; (d) in mouse brain, ICAP inhibits PKC-λ, but not PKMζ; (e) pharmacokinetic and pharmacodynamic studies of IV and PO-administered ICAP (200 mg/kg body weight) show that 24-hour integrated plasma levels of IV ICAP are 3× greater than those seen with oral per os (PO) ICAP, and, for both IV and PO administration routes, ICAP uptake over 24 hours is substantially greater in liver than in brain [21]; and (f), in toxicology studies, no abnormalities were seen with IV ICAP at a dose of 5000 mg/kg body weight [21].

### Tissue Preparations

As described [12, 13], samples from mouse brains that were flash-frozen in liquid N2 and stored at −80° C. were homogenized in buffer containing 0.25M sucrose, 20 mM Tris/HCl (pH, 7.5), 2 mM EGTA, 2 mM EDTA, 1 mM phenyl-methyl-sulfonyl-fluoride, 20 μg/ml leupeptin, 10 μg/ml aprotinin, 2 mM Na4P2O7, 2 mM Na3VO4, 2 mM NaF, and 1 μM microcystin, and then supplemented with 1% TritonX-100, 0.6% Nonidet and 150 mM NaCl.

### Western Analyses

As described [12, 13], western analyses were conducted with rabbit antisera, using: anti-aPKC [recognizes a C-terminal sequence in all aPKCs (Santa Cruz Biotechnologies, Santa Cruz, CA, USA)]; anti-phospho-threonine (thr)-560/555-PKC-ζ/λ/ι [recognizes a phosphopeptide sequence present in all aPKCs and indicates activation (Invitrogen, Carlsbad, CA, USA)]; anti-phospho-serine (ser)-473-Akt [recognizes a phosphopeptide sequence at 60 kDa present in all Akt's-1/2/3 and indicates activation (Cell Signaling Technologies, Danvers, MA, USA)]; anti-4 kDa-AP1-40/42 (Cell Signaling Technologies, Danvers, MA, USA); anti-mouse-70 kDa-BACE1 and anti-human-70 kDa-BACE1 (ThermoFisher); and anti-phospho-threonine (thr)-231-tau (Gene Tex, Irvine, CA, USA). Samples from all experimental groups within each study were compared on the same blots and routinely checked with loading controls. All comparisons in histograms were made relative to the mean value of the basal control group, i.e., wild-type mice consuming standard low-fat mouse chow, untreated with insulin or aPKC inhibitors, and having normal levels of aPKCs and other cellular proteins.

Mouse Aβ-Peptides, sAPPα, and CTFβ ELISA Analyses

Brain hemispheres were weighed and homogenized with 4 vol of phosphate-buffered saline (PBS) buffer (125 mg tissue per ml) containing a complete protease inhibitor cocktail (Sigma-Aldrich, St. Louis, MO). Half the volume of the homogenates was mixed with 8.2 mol/L guanidine-HCl (pH 7.4) (final concentration 5 mol/) for 4 hours at room temperature. Guanidine extracts were then diluted 1:50 in BSATDPBS buffer (Dulbecco's phosphate buffered saline with 5% bovine serum albumin, 0.03% Tween 20, and 1× protease inhibitor cocktail), mixed, and spun at 16,000×g for 20 minutes at 4° C. The supernatants were used to measure Aβ-peptide levels by mouse Aβ1-40 (Cat, No. 27720) and Aβ1-42 (Cat. No. 27721) ELISA kits (IBL America, Minneapolis, MN). The remainders of the brain homogenates were subsequently centrifuged at 12,000×g for 20 minutes at 4° C. Contents of sAPPα (Cat. No. 27419) and C-terminal fragment-beta (CTFO) (Cat. No. 27776) in brain tissue were measured using the IBL ELISA kits according to the manufacturer's directions.

Mouse BACE1 mRNA Analysis

RNA was extracted and purified using the RNeasy minikit (Qiagen), as described by the manufacturer. Briefly, 1p g of total RNA was used to synthesize cDNA in a 20 μl reaction mixture using the RT2 First Strand Kit for reverse transcriptase-PCR (Super Array Bioscience). The mouse BACE1 gene was amplified by using the proper primers obtained from Super Array Bioscience. β-actin was used as an internal control gene to normalize the amount of RNA. Quantitative real-time RT-PCR was performed by using Eppendorf® ep Realplex Thermal Cyclers (Eppendorf). Two μl of cDNA was added to 10 μl of SYBR Green PCR Master Mix (Applied Biosystems, CA). Each sample was run in duplicate, and analysis of relative gene expression was done by using the 2−ΔΔCt method. Briefly, the relative change in gene expression was calculated by subtracting the threshold cycle (ΔCt) of the target gene from the internal control gene. Based on the fact that the amount of cDNA doubles in each PCR cycle (assuming a PCR efficiency of 100%), the final fold-change in gene expression was calculated using the following formula: relative change=2−ΔΔCt 18.

### Memory Function Tests

Memory tests were conducted side-by-side in WT and TB/HetλKO mice consuming normal-chow.

Radial Arm Water Maze (RAWM) assesses learning and spatial memory and requires hippocampal and frontal cortical areas. The maze consisted of a circular pool filled with opaque water, with 6 arms radiating from a central area. On each training day, mice were released to swim and find a submerged escape platform at the end of a goal arm, utilizing extra-maze cues to form a spatial representation. On each day, there were 4 consecutive acquisition trials of no more than 1 minute each. For each acquisition trial, the mouse was placed in a start arm and the number of errors (arm entries that do not result in finding the platform) was recorded. If the animal made an error, it was returned to the start arm in a restart procedure. After the last acquisition trial, the animal was dried and returned to its home cage for 30 min and then given a single retention trial from the same start arm to measure short-term spatial memory.

Object Location Memory (OLM) assesses spatial and visual memory, requires hippocampal participation, and is based on the mouse's ability to learn and remember the location of a test object relative to spatial cues and recognize when the object is in a new location. Mice that remember the old spatial configuration spend increased time exploring the object in its new location. In this OLM, the experimental arena was a white Plexiglas box (23 cm×23 cm×47 cm) with visual cues marked with dark tape on 3 of the 4 walls. Mice were habituated to the arena for 5 minutes per day for 3 consecutive days. On the 4th day, the training session involved placing the animals in the arena for 5 minutes with two identical test objects placed against one wall. For the 5 minute test session, the mice were returned to the arena 3 hours later with the same objects, but with one displaced to a new location. The objects used, initial object locations, and spatial location of the displaced objects were counterbalanced between animals. The objects consisted of either a white ping-pong ball or a large hexagonal stainless steel nut of the same approximate size as the ball, which were cleaned with 25% ethanol between trials. Both training and test sessions were digitally recorded, and time spent exploring an object was measured from observation of the videos.

Exploration of the object was defined as directing the nose towards the object at a distance of 2 cm or less or touching it with the nose. Climbing or placing a paw on the object was not included unless the animal's nose was directed towards the object. A location recognition index for the test session was calculated by dividing the time spent exploring the displaced object by the total time exploring both objects.

### Statistical Analyses

Data were expressed as mean±SEM, and compared using one-way ANOVA and Tukey post hoc test for analysis of significance.

### Results

Levels of aPKCs in Brains of Wild-Type and TB/HETλKO Mice

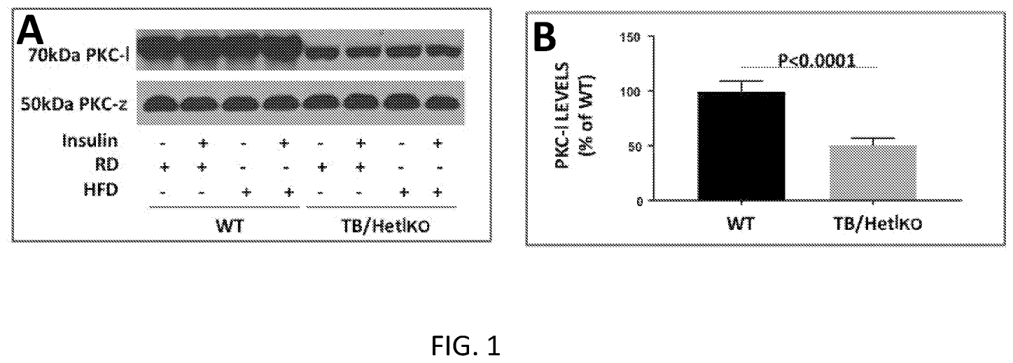

Relative to control wild-type (WT) mice, brain levels of 70 kDa aPKC were reduced by 50% in TB/HETλKO mice (FIG. 1). In marked contrast, 50 kDa aPKC levels, largely PKMζ (which, in the brain, but unlike in peripheral tissues, is produced from a separate PKMζ mRNA containing an internal promoter followed by alternative splicing) were comparable in WT and TB/HETλKO mice (FIG. 1). Thus, in the mouse brain, 70 kDa aPKC largely/exclusively reflects PKCλ/ι, and there is little or no full-length 70 kDa PKC-ζ.

Insulin Signaling to Akt and aPKC in Brains of Wild-Type and TB/HETλKO Mice

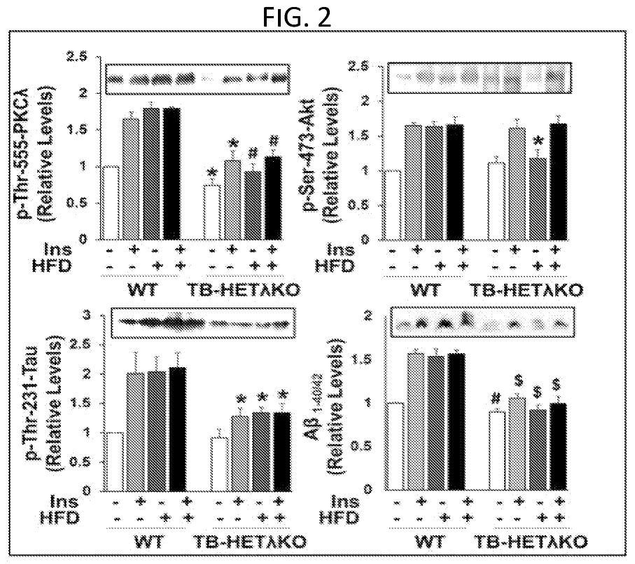

Insulin phosphorylation/activation of Akt was comparable in standard chow-fed WT and TB/HETλKO mice (FIG. 2). However, with reduced levels of brain PKC-λ/ι in TB/HETλKO mice, relative to WT mice, basal and insulin-stimulated phosphorylation/activities of brain PKC-λ/ι were diminished, regardless of diet (FIG. 2).

As discussed, serum glucose and insulin levels during glucose tolerance testing were previously shown to be elevated in presently-used HFF WT mice but normal in presently-used HFF TB/HETλKO mice [14]. In short, in hyperinsulinemic WT mice fed an HFD, basal/resting activities of both Akt and PKC-λ were elevated in the brain and unaltered by acute insulin treatment, suggesting maximal activation by pre-existing hyperinsulinemia (FIG. 2). Much differently, in normo-insulinemic TB/HETλKO mice fed an HFD, basal/resting and acute insulin-stimulated Akt activities were normal, but, with 50% deficiency of brain PKC-λ, whereas basal/resting aPKC activity was only modestly diminished, acute insulin-stimulated increases in aPKC activity were diminished by 50% or more in TB/HETλKO mice vis-á-vis stimulated activities WT mice, regardless of diet (FIG. 2).

Levels of both Aβ-peptides and phospho-thr-231-tau were elevated in HFD-fed/hyperinsulinemic WT mice, but, most importantly, these elevations were largely reduced in HFD-fed/normoinsulinemic TB/HETλKO mice (FIG. 2). Although not shown, note that total tau levels were comparable in WT and TB/HETλKO mice and were not altered by diet or acute insulin treatment. We did not evaluate other phosphorylation sites of tau.

Levels of Aβ1-40/42, Aβ40, Aβ42, CTFβ, sAPPα and Phospho-Thr-231-Tau, in Brains of Wild-Type and TB/HETλKO Mice

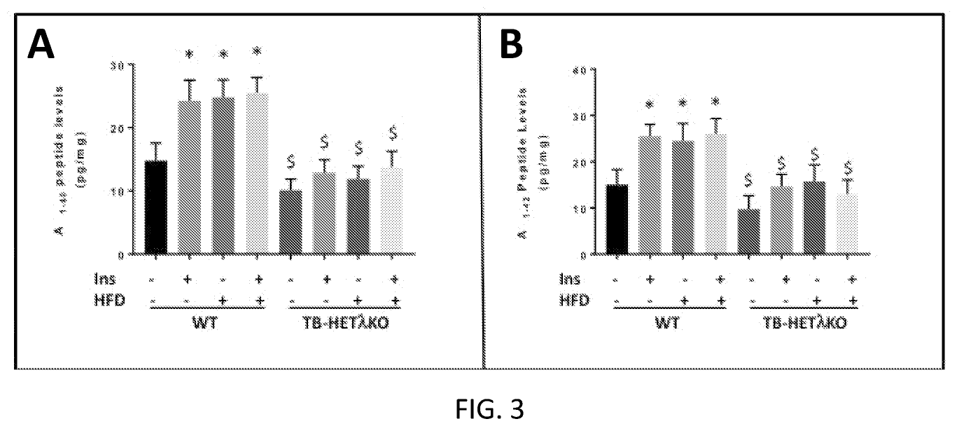

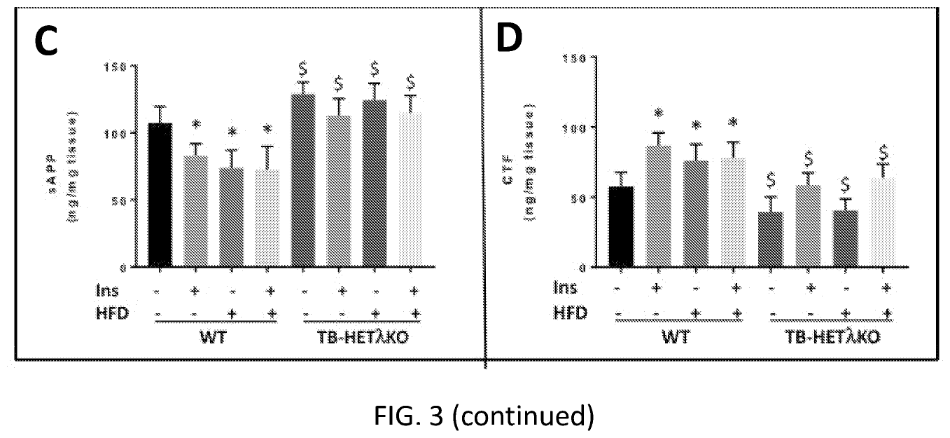

In addition to decreases in 4 kDa total Aβ1-40/42 levels seen in western analyses, brain levels of Aβ40 and Aβ42, as measured separately by ELISA, were elevated by acute insulin treatment and HFD-induced chronic hyperinsulinemia in WT mice, but not in TB/HETλKO mice (FIGS. 3a and 3b). Further, in keeping with increases in Aβ-peptides, and, as reflective of the proteolytic action of BACE1 on βAPP, there were insulin and HFD/hyperinsulinemia-induced increases in brain levels of the C-terminal fragment of βAPP (CTFβ, or “C99”) in WT, but not TB/HETλKO mice (FIG. 3d); also, as might be expected, the alterations in brain CTFβ levels were accompanied by apparently reciprocal alterations in αsecretase-dependent generation of the N-terminal secreted/soluble fragment of βAPP (sAPPα) (FIG. 3c).

Protein and mRNA Levels of BACE1 in Brains of Wild-Type and TB/HETλKO Mice

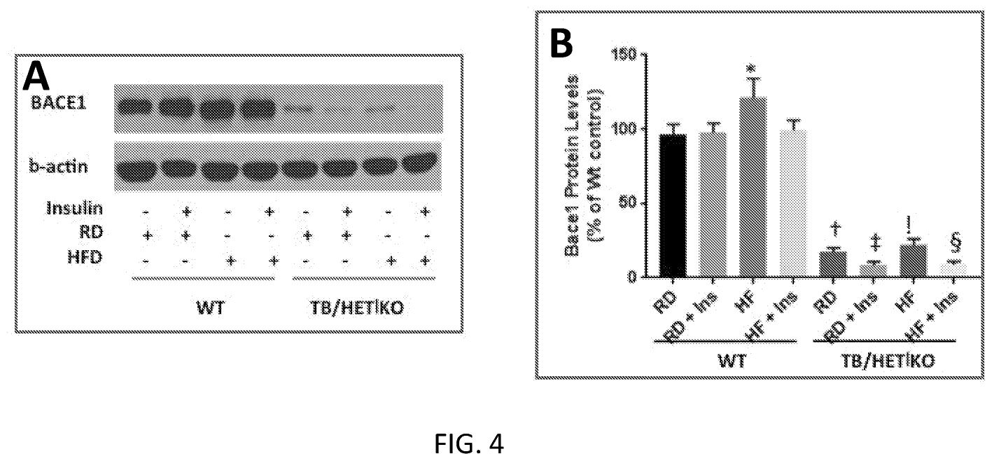

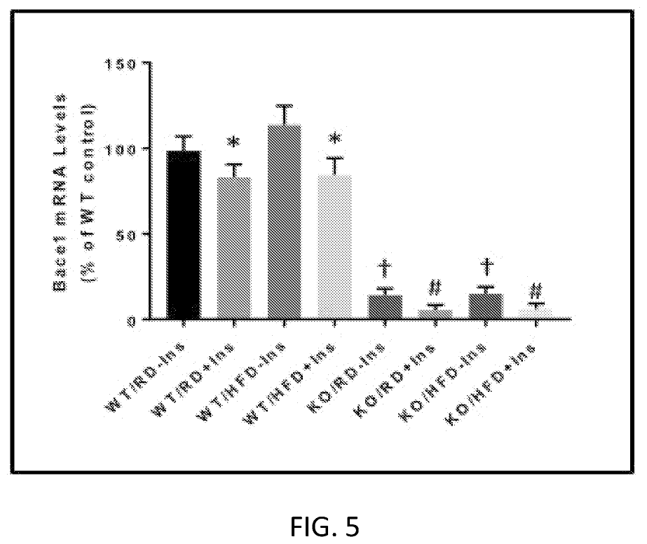

In WT mice fed an HFD with elevated brain PKC-λ/ι activity, basal/resting levels of BACE1 protein were increased (FIG. 4). In contrast, in TB/HETλKO mice fed an HFD, with reduced activity of brain PKC-λ/ι, basal/resting protein levels of BACE1 protein, Aβ1-40/42, and phospho-thr-231-tau were markedly diminished (FIG. 4). Interestingly, acute insulin treatment further reduced BACE1 protein levels in TB/HETλKO mice (FIG. 4).

### Effects of High-Fat-Feeding and PKC-λ Haploinsufficiency on Activities of IKKα/β and NFκB, and Levels of Proinflammmatory Cytokines, TNF-1α, and IL-1β, in Brains of Wild-Type and TB/HETλKO Mice

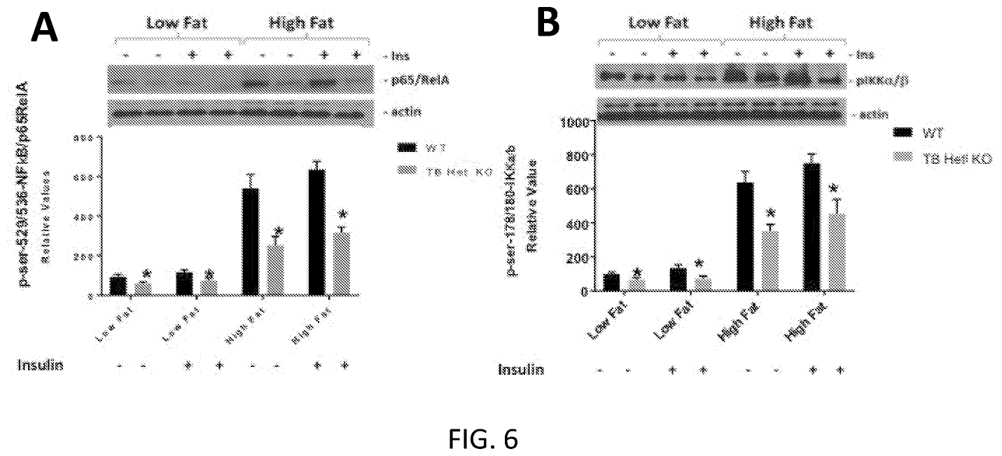

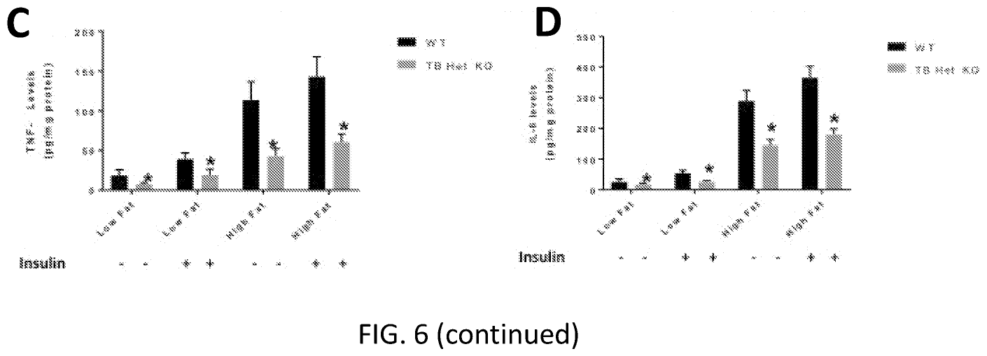

We previously found: (a) that hyperinsulinemia leads to aPKC-dependent increases in hepatic activity of NFκB and its activator, inhibitor of the kappa-B kinase (IKK) in the liver [22, 23]; and (b) there are increases in proinflammatory cytokine expression in livers of HFF mice [15] and ob/ob mice [24], and hepatocytes of T2D humans [20]. Here, we found that hyperinsulinemia similarly provokes increases in activities of IKK and NFκB, as well as increases in levels of TNF-α and IL-6, in brains of WT mice; moreover, all of these HFD-induced increases in IKK/NFκB activities and cytokine levels were reduced by approximately 50% in mice with PKC-λ haploinsufficiency (FIG. 6). Note that these decreases in HFD-induced increases in brain cytokine levels in mice with PKC-λ haploinsufficiency are similar to the decreases in normal-feeding/insulin-induced increases in hepatic cytokine expression seen in livers of mice with PKC-λ haploinsufficiency [14].

### Learning/Memory Functions in Wild-Type and TB/HETλKO Mice

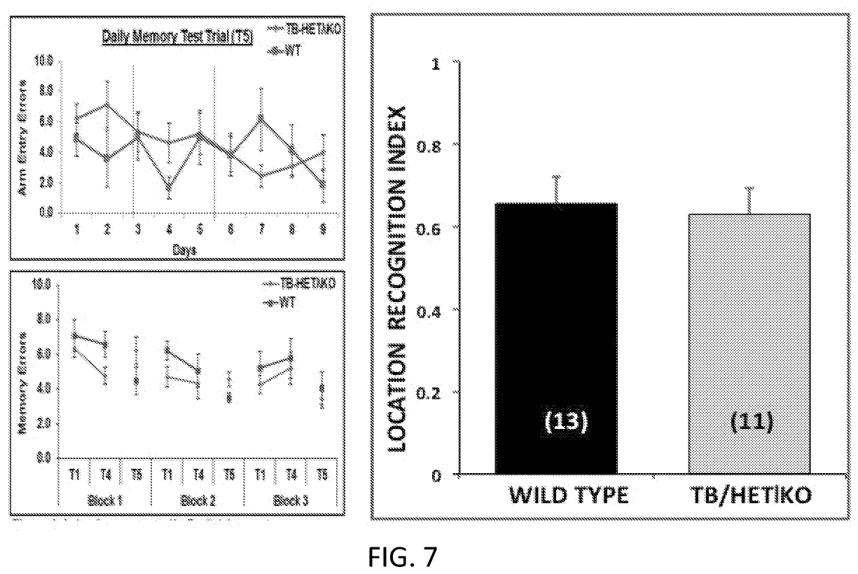

Despite partially reduced brain PKC-λ/ι levels and markedly reduced BACE1 levels, learning/memory functions in Radial Arm Water Maze and Object Location Memory tests were comparable in TB/HETλKO and WT mice (FIG. 7).

### Effects of Nasally Administered PKC-λ/ι Inhibitor on BACE1 Levels in Brains and Livers of Wild-Type Mice

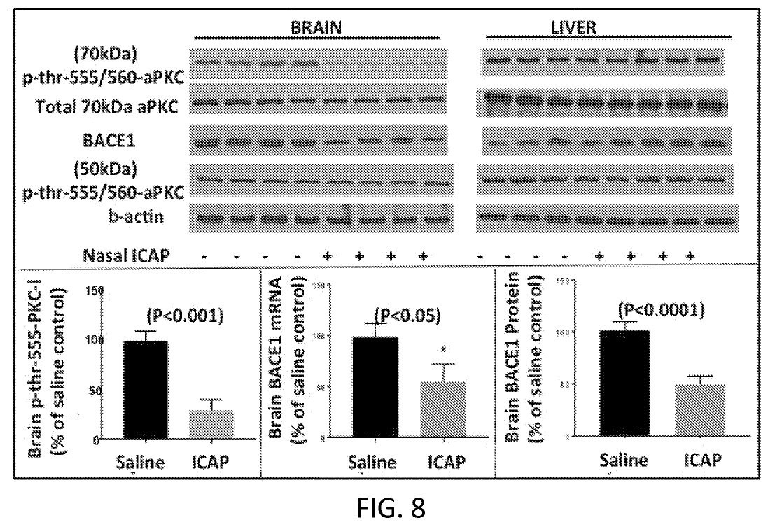

To explain the deficiency of BACE1 in TB/HETλKO mice, it could be argued that this deficiency was an indirect adaptive mechanism to life-long deficiency of PKC-λ/ι. However, as clear evidence that BACE1 depletion in TB/HetλKO mice resulted directly from the deficiency of brain PKC-λ/ι, we found that nasally-administered PKC-λ/ι inhibitor ICAP over a 3-week period (a) inhibited 70 kDa PKC-λ/ι activity in the brain by 70-80%, but not in liver (the main target of IV and PO-administered ICAP [21]; and see below), and (b) simultaneously diminished brain BACE1 protein and mRNA levels by approximately 50% (FIG. 7). In contrast, brain 50 kDa PKMζ activity was not altered by this level of ICAP treatment, reflecting the aPKC isoform selectivity of ICAP (FIG. 8).

### Effects of Intravenously Administered PKC-λ/ι Inhibitor on BACE1 Levels in Brains and Livers of Wild-Type Mice

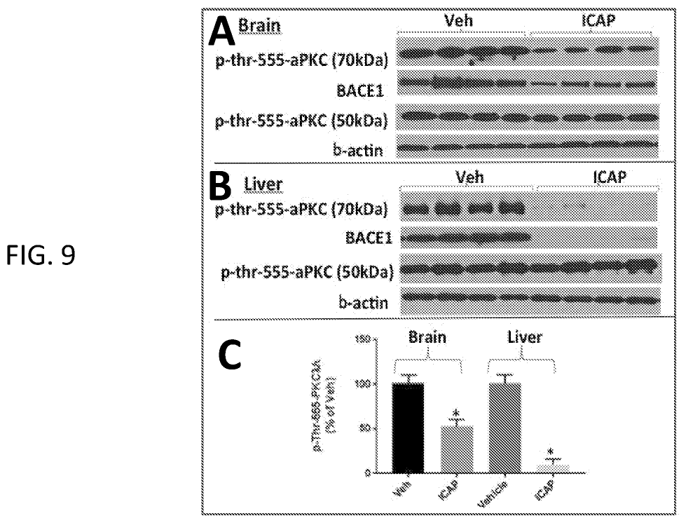

Very different from nasally-administered ICAP, IV-administered ICAP markedly inhibited liver PKC-λ/ι activity and concomitantly diminished liver BACE1 levels by approximately 90% (FIG. 9). As might be expected from pharmacokinetic and pharmacodynamic studies (which showed a greater distribution of ICAP in the liver than in the brain during a 24-hour period after IV and oral administration [21]), IV-administered ICAP diminished brain PKC-λ/ι activity and brain BACE1 levels by approximately 50-60% (FIG. 9).

### Effects of Insulin on NFκB Activity and BACE1 Levels in Cultured Neuronal Cells

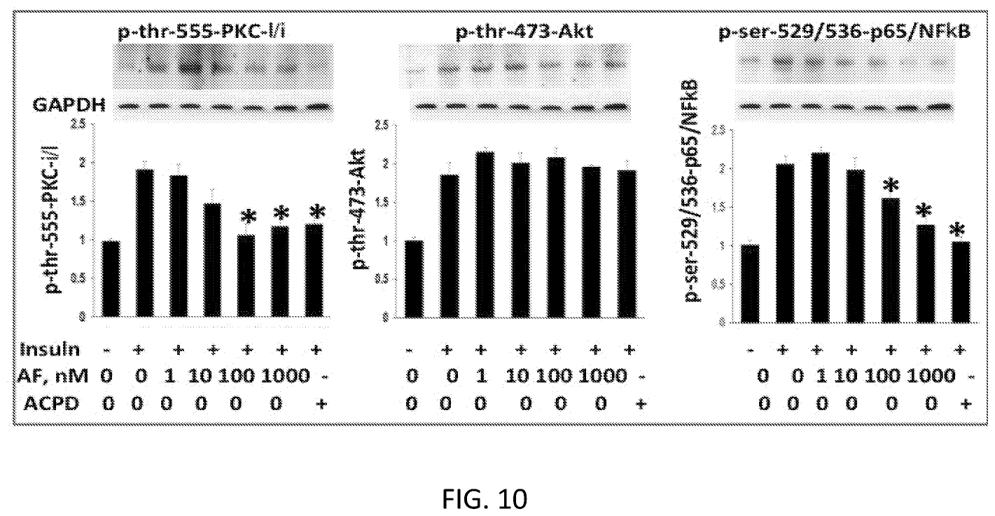

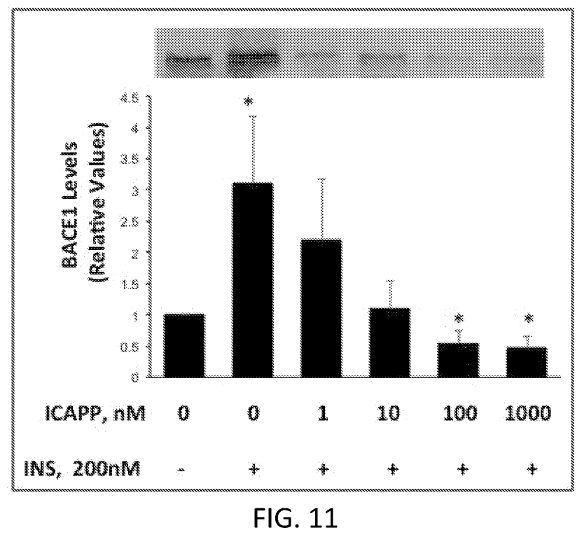

As hyperinsulinemia-induced increases in NFκB activity and BACE1 levels in brains of WT mice could be due to factors other than insulin that similarly act via, or dependent on, PKC-λ/ι, it was important to find that NFκB activity (FIG. 10) and BACE1 levels (FIG. 11) were increased by 24 hour insulin treatment of LA1-5s human neuroblastoma neuronal cells. Moreover, these effects of insulin (but not Akt activation) were diminished by treatment with several PKCλ/ι inhibitors, including ICAPP, which, at higher levels also reduced basal/resting levels of BACE1, and both auranofin (AF; which inhibits the regulatory domain of aPKC [26]) and ACPD (which, like ICAPP, inhibits the catalytic domain of aPKC [15, 19]) (FIGS. 10 and 11). Note that we previously reported that insulin increases PKC-λ/ι activity, BACE1 activity, and production of Aβ1-40/42 in LA1-5s neuronal cells, and these effects of insulin, but not Akt activation, are similarly blocked by ICAPP [13].

### Discussion

The presently observed protection against developing HFD-induced increases in brain Akt activity in TB/HETλKO mice vis-à-vis WT mice is best explained by the failure to develop hyperinsulinemia owing to hepatic deficiency of PKCλ/ι and diminished gluconeogenesis observed previously in TB/HETλKO mice [14]. On the other hand, the protection against developing HFD-induced increases in brain aPKC activity in TB/HETλKO mice may be explained by either or both: (a) failure to develop hyperinsulinemia in TB/HETλKO mice; and (b) deficiency of brain PKC-λ/ι, which diminished acute effects of insulin on brain PKC-λ/ι activity in TB/HETλKO mice, regardless of diet.

The effects of chronic hyperinsulinemia on brain BACE1 levels in HFF mice may reasonably be construed to be largely due to effects of insulin itself, is supported by the present finding that simple 24-hour treatment of cultured neuronal cells with insulin provoked marked increases in BACE1 levels that were similar to, or exceeded, increases in PKC-λ/ι activity, BACE1 activity, and Aβ1-40/42 levels reported previously [13]. Further, as insulin activation of brain Akt was conserved in both brains of TB/HETλKO mice and in cultured neuronal cells, and, as deficiency and/or chemical inhibition of PKC-λ/ι diminished BACE1 levels in both experimental models, it may reasonably be surmised that deficiencies in levels and/or activity of PKC-λ, rather than Akt, in brains of TB/HETλKO mice and cultured neuronal cells accounted for decreases in BACE1 levels, which in turn accounted for decreases in brain levels of Aβ1-40/42. On the other hand, how deficiency or inhibition of PKC-λ/ι led to decreases in p-thr-231-tau remains uncertain.

The mechanism underlying the large reduction of BACE1 protein levels in TB/HETλKO mice was found to involve a comparably large reduction in BACE1 mRNA levels. These decreases in mRNA suggested that PKC-λ/ι is required for maintaining BACE1 transcription, a process known to be activated by NFκB [6-8]. Indeed, we found that HFD provoked increases in (a) activities of NFκB and its activator, IKK, and (b) NFκB-dependent increases in expression of proinflammatory cytokines, TNF-α and IL-1β; moreover, these effects were reduced by approximately 50%, in accordance with decreases in PKC-λ/ι levels in TB/HETλKO mice. Similarly, in cultured neuronal cells, insulin provoked increases in IKKα/β and NFκB, and a variety of inhibitors of PKC-λ/ι blocked these effects. Together, these findings suggest that, in the brain, insulin increases the expression of BACE1 and proinflammatory cytokines by sequential activation of PKC-λ/ι, IKKα/β, and NFκB.

However, in addition to insulin as an activator of NFκB and the inflammatory pathway, it is interesting to note that oxidative and inflammatory processes are activated by supraphysiological levels of AD, as well as a wide variety of factors [2, 5]. Increases in BACE1 transcription in the brain are also operative with metformin treatment [27], which we found, like insulin, increases aPKC activity in both cultured neuronal cells [13] and mouse brain (Sajan and Farese, unpublished work on chronic metformin treatment), as well as in liver [19] and muscle [28].

However, it is also possible that decreases in BACE1 longevity may have contributed to the decreases in BACE1 protein levels in TB/HETλKO mice. In this regard, as reported [29], phosphorylation of ser-498 on the BACE1 cytoplasmic tail appears to be PKC-λ/ι-dependent, and this phosphorylation increases trafficking of BACE1-containing vesicles to the trans-Golgi network (TGN), i.e., away from lysosomes where BACE1 is destroyed. Indeed, BACE1 half-life is increased by this PKC-λ/ι-dependent trafficking [29], which may also explain: (a) acute insulin-dependent increases in BACE1 activity in mouse brain reported previously [13], perhaps owing to TGN acidity; and (b) increases in Aβ1-40/42 production, resulting from BACE1 activation enhanced interaction between BACE1-containing and βAPP-containing vesicles in the TGN [1, 2, 4, 30]. Further studies are needed to evaluate and quantify various mechanisms responsible underlying the dependence of increases in activity and levels of BACE1 on PKC-λ/ι.

BACE1 protein levels were increased by a 10-week HFD in WT mice, which suggested that chronic hyperinsulinemia-induced PKC-λ/ι activation leads to increased production and/or longevity of BACE1. Surprisingly, however, acute insulin treatment caused HFD-stimulated BACE1 protein levels to trend downward in WT mice and caused already-suppressed BACE1 levels to significantly diminish further in TB/HETλKO mice. These findings suggest that, particularly with PKC-λ/ι deficiency and diminished ability to maintain BACE1 protein levels and trafficking of BACE1-containing vesicles to the TGN in TB/HETλKO mice, such trafficking may increase the movement of these BACE1-containing vesicles to lysosomes where BACE1 is degraded. Lysosomal trafficking may also occur following the trafficking of BACE1 and βAPP-containing vesicles from the TGN to the plasma membrane where AR-peptides are released following γ-secretase action on CTFβ during insulin activation of mitogen-activated protein kinase [31].

In addition to acute decreases in BACE1 protein levels, BACE1 mRNA levels surprisingly fall with acute insulin treatment. These acute decreases in BACE1 mRNA most likely reflect increases in degradative turnover in response to acute stimulation with supramaximal insulin. Further studies are needed to see if mRNA turnover, as well as synthesis, is increased in hyperinsulinemic mice.

It was interesting to find that generation of brain levels of CTFβ were increased acutely by insulin and chronically by HFD/hyperinsulinemia in WT mice, and these increases in CTFβ were substantially reduced in TB/HETλKO mice that were deficient in brain levels of PKC-λ and BACE1; moreover, all changes in CTFβ were accompanied by reciprocal alterations in sAPPα. This reciprocity during alterations in BACE1 action suggests that activity of the BACE1 dependent amyloidogenic pathway (which generates Aβ-peptides) can limit the operation of the α-secretase-dependent non-amyloidogenic pathway (which generates sAPPα), perhaps by altering βAPP substrate availability. In keeping with the latter postulation, increases in activity of α-secretase-dependent pathway by phorbol ester/diacylglycerol (DAG)-sensitive PKCs can limit BACE1 action and reduce Aβ-peptide production [32-34]. The contrasting effects of DAG-sensitive PKCs (most notably, PKC-α and PKC-ε) and DAG-insensitive/PIP3-sensitive aPKCs (most notably, PKC-λ/ι) on α and β-secretases, respectively, and thus on Aβ1-40/42 production, underscore the functional specificity of PKC isoforms on βAPP proteolysis and provide new approaches for regulation of Aβ production.

The normal activation of brain Akt by insulin in TB/HETλKO mice is noteworthy, as insulin activation of IR, IR substrates 1 and 2 (IRS-1 and IRS-2), and IRS-1/2-dependent PI3K needed for Akt activation are all markedly reduced in the liver, muscle, and adipose tissues of TB/HETλKO mice, presumably compensating (teleologically to avoid hypoglycemia) for the constitutive phosphorylation/inactivation of hepatic FoxO1, and subsequent decreases in gluconeogenic enzyme expression were seen in these mice [14]. Conserved brain IR/Akt signaling may reflect structural differences between type-A IRs in the brain and type-B IRs in the liver, muscle, and adipose tissues, and/or differences in activity mediated by alterations in the phosphorylation status of the IR or IRS1/2.

It was, of course, important to find that learning/memory functions in RAWM and OLM tests were comparable in WT and TB/HETλKO mice. This suggests that only moderate levels of PKC-λ/ι activity, and little if any BACE1, is sufficient for these functions. The latter point is in keeping with the observance of improvement in long term potentiation and learning/memory function (contextual fear conditioning test) in transgenic 5×FAD mice following conditional knockout of BACE1 [35] and BACE1 haplo-insufficiency [36]. The agreement of these findings raises the possibility that BACE1 depletion may not be attended by the cognitive/memory deficits that were seen with chemical BACE1 inhibitors in recently-aborted human clinical trials.

The placement of BACE1, Aβ1-40/42, and phospho-thr-231-tau downstream of PKC-λ/ι has widespread importance, as aPKC is activated not only by insulin but also by various AD risk factors, including ceramide [15], phosphatidic acid [37], hyperglycemia [38] and proinflammatory cytokines, most notably, tumor necrosis factor-α (TNF-α) and interleukin-1β [39]. Thus, aPKC activation may mediate or contribute to AD risks imposed by these and other factors.

It was of considerable interest that, similar to the marked decreases in brain BACE1 levels following nasal administration of ICAP and selective blockade of brain PKC-λ/ι, the marked inhibition of hepatic PKC-λ/ι by IV-administered ICAP was accompanied by marked decreases in hepatic BACE1 levels. This similarity suggests that BACE1 is fully dependent upon, and tightly regulated by, PKC-λ/ι throughout the body. Moreover, the effects of IV ICAP on liver BACE1 may be particularly important, as it was recently reported that BACE1 levels are elevated in the T2D liver and serve to diminish hepatic IR levels [40]. It may therefore be surmised that the excessive activation of hepatic PKC-λ/ι and subsequent decreases in hepatic IR levels contribute to the development of hepatic and systemic insulin resistance in obesity and T2D, i.e., along with PKC-λ/ι-dependent impairments in (a) Akt actions on FoxO1 and PGC-1α [15, 16]; and (b) activation of IRS-1-dependent PI3K [20]. Moreover, our initial unpublished findings suggest that in both liver and brain, HFD-induced increases in PKC-λ/ι activity are accompanied by increases in BACE1 levels and reciprocal decreases in IR levels, and all changes are reversed by inhibition of hepatic PKC-λ/ι.

We found out that (a) nasal ICAP selectively inhibited brain (not liver) PKC-λ/ι, and (b) IV ICAP inhibited both liver and brain PKC-λ/ι, which may have additional therapeutic implications, whereas treatment of non-diabetic AD may be better served by the use of nasal PKC-λ/ι inhibitors that target only brain PKC-λ/ι, the combined presence of obesity/T2D and AD may be better served by the use of PKC-λ/ι inhibitors that are administered by oral (PO) or subcutaneous (SC) routes and therefore target both liver and brain PKC-λ/ι. Obviously, more studies in experimental animals are needed to determine (a) the lowest doses of nasal, SC and PO ICAP, and other PKC-λ/ι inhibitors needed for effective treatment of T2D and AD, and (b) the safety of these doses in long-term studies. In this regard, it is worth noting that the T2D state in both HFF [15, 16] and ob/ob [24] mice is substantially improved by relatively low doses of PKC-λ/ι inhibitors that produce only modest decreases (i.e., to essentially normal levels) in basal and insulin-stimulated activities of hepatic PKC-λ/ι. It will be interesting to see if reduction of BACE1 to normal, rather than to subnormal, levels is sufficient to effectively improve learning/memory functions and prevent AD pathology development during long-term treatment of mouse AD models (now in progress).

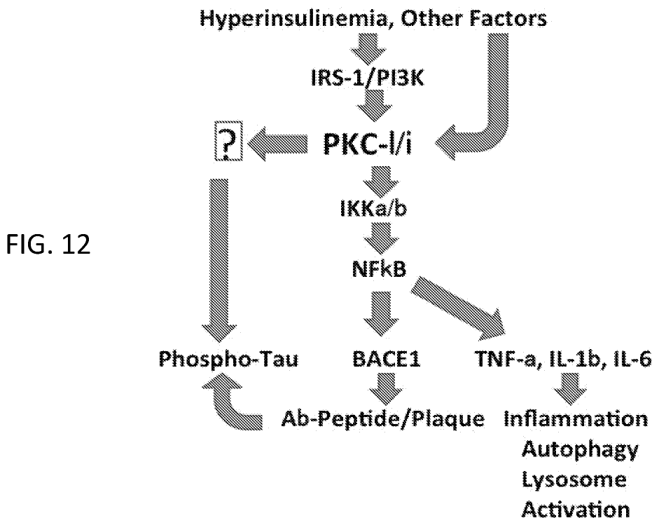

To summarize, we provide both gene knockout-based and chemical inhibitor-dependent evidence that brain PKCλ/ι is required for maintenance of BACE1 mRNA and protein levels, and associated increases in Aβ1-40/42 and p-thr-231 in hyperinsulinemic states of obesity/T2D (proposed pathways are portrayed in FIG. 12). Moreover, the marked dependence of basal BACE1 levels on PKC-λ/ι activity in both brain and liver suggests that PKC-λ/ι plays a major and widespread role in BACE1 regulation. Additionally, we show that brain PKC-λ/ι activity and BACE1 levels can be selectively controlled by nasal application of a PKC-λ/ι inhibitor and simultaneously controlled in the liver and brain by systemic administration of inhibitor. These findings may have relevance for devising treatment of non-diabetic AD and AD associated with obesity/T2D. Further studies are needed to see if reduction of brain BACE1 levels by aPKC inhibition can safely serve as an effective approach for improving functional and pathological abnormalities of AD.

### REFERENCES

1. Cole S L, Vassar R. The Alzheimer's disease beta-secretase enzyme, BACEL. Mol Neurodegener 2007; 2: 22-70. http://dx.doi.org/10.1186/1750-1326-2-22 PMID: 18005427
2. Kandalepas P C, Vassar R. The normal and pathologic roles of the Alzheimer's (3-secretase, BACE1. Curr Alzheimer Res 2014; 11(5): 441-9. http://dx.doi.org/10.2174/1567205011666140604122059 PMID: 24893886
3. Yang L-B, Lindholm K, Yan R, et al. Elevated beta-secretase expression and enzymatic activity detected in sporadic Alzheimer disease. Nat Med 2003; 9(1): 3-4. http://dx.doi.org/10.1038/nm0103-3 PMID: 12514700
4. Tan J, Evin G. B-site APP-cleaving enzyme 1 trafficking and Alzheimer's disease pathogenesis. J Neurochem 2012; 120(6): 869-80. http://dx.doi.org/10.1111/j.1471-4159.2011.07623.x
5. Chami L, Checler F. BACE1 is at the crossroad of a toxic vicious cycle involving cellular stress and β-amyloid production in Alzheimer's disease. Mol Neurodegener 2012; 7: 52-67. http://dx.doi.org/10.1186/1750-1326-7-52 PMID: 23039869
6. Buggia-Prevot V, Sevalle J, Rossner S, Checler F. NFkappaB-dependent control of BACE1 promoter transactivation by Abeta42. J Biol Chem 2008; 283(15): 10037-47. http://dx.doi.org/10.1074/jbc.M706579200 PMID: 18263584
7. Chen C-H, Zhou W, Liu S, et al. Increased NF-κB signalling upregulates BACE1 expression and its therapeutic potential in Alzheimer's disease. Int J Neuropsychopharmacol 2012; 15(1): 77-90. http://dx.doi.org/10.1017/S1461145711000149 PMID: 21329555
8. Wang R, Chen S, Liu Y, et al. All-trans-retinoic acid reduces BACE1 expression under inflammatory conditions via modulation of nuclear factor KB (NFκB) signaling. J Biol Chem 2015; 290(37): 22532-42. http://dx.doi.org/10.1074/jbc.M115.662908 PMID: 26240147
9. Janson J, Laedtke T, Parisi J E, O'Brien P, Petersen R C, Butler P C. Increased risk of type 2 diabetes in Alzheimer disease. Diabetes 2004; 53(2): 474-81. http://dx.doi.org/10.2337/diabetes.53.2.474 PMID: 14747300
10. Talbot K, Wang Y, Kazi H, et al. Demonstrated brain insulin resistance, IRS-1 dysregulation, and cognitive decline. J Clin Invest 2012; 122: 1316-38. http://dx.doi.org/10.1172/JCI59903 PMID: 22476197
11. Benedict C, Grillo C A. Insulin resistance as a therapeutic target in the treatment of Alzheimer's disease; A state-of-the-art review. Front Neurosci 2018; 12: 215-28. http://dx.doi.org/10.3389/fnins.2018.00215 PMID: 29743868
12. Sajan M, Hansen B, Ivey R III, et al. Brain insulin signaling is increased in insulin-resistant states and decreases in FoxOs and PGC-1α and increases in Aβ1-40/42 and phospho-tau may abet Alzheimer's development. Diabetes 2016; 65(7): 1892-903. http://dx.doi.org/10.2337/dbl5-1428 PMID: 26895791
13. Sajan M P, Hansen B C, Higgs M G, et al. Atypical PKC, PKCO/A, activates β-secretase and increases Aβ1-40/42 and phospho-tau in mouse brain and isolated neuronal cells, and may link hyperinsulinemia and other aPKC activators to development of pathological and memory abnormalities in Alzheimer's disease. Neurobiol Aging 2018; 61: 225-37. http://dx.doi.org/10.1016/j.neurobiolaging.2017.09.001 PMID: 29032894
14. Sajan M P, Ivey R A III, Lee M, et al. PKCX haploinsufficiency prevents diabetes by a mechanism involving alterations in hepatic enzymes. Mol Endocrinol 2014; 28(7): 1097-107. http://dx.doi.org/10.1210/me.2014-1025 PMID: 24877563
15. Sajan M P, Acevedo-Duncan M E, Standaert M L, Ivey R A III, Lee M C, Farese R V. Akt-dependent phosphorylation of hepatic Fox01 is compartmentalized on a WD40/Propeller/FYVE scaffold and is selectively inhibited atypical PKC in early phases of diet-induced obesity. A mechanism for impairing gluconeogenic but not lipogenic enzyme expression. Diabetes 2014; 63: 2690-701. http://dx.doi.org/10.2337/dbl3-1863 PMID: 24705403
16. Sajan M P, Lee M C, Foufelle F, Sajan J, Cleland C, Farese R V. Coordinated regulation of hepatic FoxO1, PGC-1α and SREBP-1c facilitates insulin action and resistance. Cell Signal 2018; 43: 62-70. http://dx.doi.org/10.1016/j.cellsig.2017.12.005 PMID: 29269047
17. Wang S, Sheng T, Ren S, Tian T, Lu W. Distinct roles of PKCi/k and PKM in the initiation and maintenance of hippocampal longterm potentiation and memory. Cell Rep 2016; 16(7): 1954-61. http://dx.doi.org/10.1016/j.celrep.2016.07.030 PMID: 27498875
18. Sheng T, Wang S, Qian D, Gao J, Ohno S, Lu W. Learning-induced suboptimal compensation for PKCi/k function in mutant mice. Cereb Cortex 2017; 27(6): 3284-93. http://dx.doi.org/10.1093/cercor/bhx077 PMID: 28383692
19. Sajan M P, Ivey R A III, Farese R V. Metformin action in human hepatocytes: coactivation of atypical protein kinase C alters 5′-AMP-activated protein kinase effects on lipogenic and gluconeogenic enzyme expression. Diabetologia 2013; 56(11): 2507-16. http://dx.doi.org/10.1007/s00125-013-3010-1 PMID: 23933835
20. Sajan M P, Farese R V. Insulin Signalling in Hepatocytes of Type 2 Diabetic Humans. Excessive Expression and Activity of PKC-ι and Dependent Processes and Reversal by PKC-ι Inhibitors. Diabetologia 2012; 55: 1446-57. http://dx.doi.org/10.1007/s00125-012-2477-5 PMID: 22349071
21. Apostolatos A H, Apostolatos C A, Ratnayake W S, et al. Preclinical testing of 5-amino-1-((1R,2S,3S,4R)-2,3-dihydroxy-4-methylcyclopentyl)-1H-imidazole-4-carboxamide: A potent protein kinase C-ι inhibitor as a potential prostate carcinoma therapeutic. Anticancer Drugs 2019; 30(1): 65-71. http://dx.doi.org/10.1097/CAD.0000000000000694 PMID: 30204596
22. Sajan M P, Standaert M L, Nimal S, et al. The critical role of atypical protein kinase C in activating hepatic SREBP-1c and NFkappaB in obesity. J Lipid Res 2009; 50(6): 1133-45. http://dx.doi.org/10.1194/jlr.M800520-JLR200 PMID: 19202134
23. Sajan M P, Standaert M L, Rivas J, et al. Role of atypical protein kinase C in activation of sterol regulatory element binding protein-1c and nuclear factor kappa B (NFkappaB) in liver of rodents used as a model of diabetes, and relationships to hyperlipidaemia and insulin resistance. Diabetologia 2009; 52(6): 1197-207. http://dx.doi.org/10.1007/s00125-009-1336-5 PMID: 19357831
24. Sajan M P, Ivey R A, Lee M C, Farese R V, Lee M C, Farese R V. Hepatic insulin resistance in ob/ob mice involves increases in ceramide, aPKC activity, and selective impairment of Akt-dependent Fox01 phosphorylation. J Lipid Res 2015; 56(1): 70-80. http://dx.doi.org/10.1194/jlr.M052977 PMID: 25395359
25. Hemandez A I, Blace N, Crary J F, et al. Protein kinase M zeta synthesis from a brain mRNA encoding an independent protein kinase C zeta catalytic domain. Implications for the molecular mechanism of memory. J Biol Chem 2003; 278(41): 40305-16. http://dx.doi.org/10.1074/jbc.M307065200 PMID: 12857744
26. Erdogan E, Lamark T, Stallings-Mann M, et al. Aurothiomalate inhibits transformed growth by targeting the PB1 domain of protein kinase Ciota. J Biol Chem 2006; 281(38): 28450-9. http://dx.doi.org/10.1074/jbc.M606054200 PMID: 16861740
27. Chan Y, Zhou K, Wang R, et al. Antidiabetic drug metformin (GlucophageR) increases biogenesis of Alzheimer;s amyloid peptides via up-regulating BACE1 transcription. Proc Natl Acad Sci USA 2009; 106: 33907-12.
28. Sajan M P, Bandyopadhyay G, Miura A, et al. AICAR and metformin, but not exercise, increase muscle glucose transport through AMPK-, ERK-, and PDK1-dependent activation of atypical PKC. Am J Physiol Endocrinol Metab 2010; 298(2): E179-92. http://dx.doi.org/10.1152/ajpendo.00392.2009 PMID: 19887597
29. Sun M, Zhang H. Par3 and aPKC regulate BACE1 endosome-to-TGN trafficking through PACS1. Neurobiol Aging 2017; 60: 129-40. http://dx.doi.org/10.1016/j.neurobiolaging.2017.08.024 PMID: 28946017
30. Vieira S I, Rebelo S, Esselmann H, et al. Retrieval of the Alzheimer's amyloid precursor protein from the endosome to the TGN is S655 phosphorylation state-dependent and retromer-mediated. Mol Neurodegener 2010; 5: 40-61. http://dx.doi.org/10.1186/1750-1326-5-40 PMID: 20937087 Gasparini L, Gouras G K, Wang R, et al. Stimulation of β-amyloid precursor protein trafficking by insulin reduces intraneuronal Pamyloid and requires mitogen-activated protein kinase signaling. J Neurosci 2001; 21(8): 2561-70. http://dx.doi.org/10.1523/JNEUROSCI.21-08-02561.2001 PMID: 11306609 31. Buxbaum J D, Gandy S E, Cicchetti P, et al. Processing of Alzheimer beta/A4 amyloid precursor protein: modulation by agents that regulate protein phosphorylation. Proc Natl Acad Sci USA 1990; 87(15): 6003-6. http://dx.doi.org/10.1073/pnas.87.15.6003 PMID: 2116015
32. Sinha S, Lieberburg I. Cellular mechanisms of 0-amyloid production and secretion. Proc Natl Acad Sci USA 1999; 96(20): 11049-53. http://dx.doi.org/10.1073/pnas.96.20.11049 PMID: 10500121 Nunan J, Small D H. Regulation of APP cleavage by α-, β and γsecretases. FEBS Lett 2000; 483(1): 6-10. http://dx.doi.org/10.1016/S0014-5793(00)02076-7 PMID: 11033346
33. Hu X, Das B, Hou H, He W, Yan R. BACE1 deletion in the adult mouse reverses preformed amyloid deposition and improves cognitive functions. J Exp Med 2018; 215(3): 927-40. http://dx.doi.org/10.1084/jem.20171831 PMID: 29444819
34. Kimura R, Devi L, Ohno M. Partial reduction of BACE1 improves synaptic plasticity, recent and remote memories in Alzheimer's disease transgenic mice. J Neurochem 2010; 113(1): 248-61. http://dx.doi.org/10.1111/j.1471-4159.2010.06608.x PMID: 20089133
35. Sajan M P, Bandyopadhyay G, Kanoh Y, et al. Sorbitol activates atypical protein kinase C and GLUT4 glucose transporter translocation/glucose transport through proline-rich tyrosine kinase-2, the extracellular signal-regulated kinase pathway and phospholipase D. Biochem J 2002; 362(Pt 3): 665-74. http://dx.doi.org/10.1042/bj3620665 PMID:
36. Bandyopadhyay G, Sajan M P, Kanoh Y, et al. Glucose activates protein kinase C-ζ/λ, through proline-rich tyrosine kinase-2, extracellular signal-regulated kinase, and phospholipase D: A novel mechanism for activating glucose transporter translocation. J Biol Chem 2001; 276(38): 35537-45. http://dx.doi.org/10.1074/jbc.M106042200 PMID: 11463795
37. Anthonsen M W, Andersen S, Solhaug A, Johansen B. Atypical λ/ι PKC conveys 5-lipoxygenase/leukotriene B4-mediated cross-talk between phospholipase A2s regulating NF-κ B activation in response to tumor necrosis factor-α and interleukin-1β. J Biol Chem 2001; 276(38): 35344-51. http://dx.doi.org/10.1074/jbc.M105264200 PMID: 11445585 38. Meakin P J, Mezzapesa A, Benabou E, et al. The beta secretase BACE1 regulates the expression of insulin receptor in the liver. Nat Commun 2018; 9(1): 1306. http://dx.doi.org/10.1038/s41467-018-03755-2 PMID: 29610518

### Example 2

### Background

Alzheimer's disease (AD) afflicts 1 in 5 women and 1 in 10 men over age 65, and 45% of people over age 85. Lewy Body disease (LBD), which has features of AD and Parkinson's disease, and Frontotemporal dementia (FTD), the most common hereditary dementia, are also on the rise. Although etiologies differ and pathologies vary in location and kind, inflammation is a common feature that may be abetted by hyperinsulinemia in insulin-resistant forms of obesity (O), the metabolic syndrome (MetS) and T2D, which together afflicts over 50% of our population, and have been reported to be risk factors for development of AD (1-4), LBD/PD (5,6) and FTD (7,8).

To date, attempts to develop effective treatments for these deadly neurodegenerative disorders have been unsuccessful. Accordingly, these disorders are approaching epidemic levels as our population ages, and their cost, both in dollars and tragic morbidity, is staggering. Obviously, stemming this epidemic is one of our greatest challenges. To this end, we have identified a major inflammatory signaling pathway that is uniquely operative in brain, insulin-sensitive, and excessively activated in human AD and mouse models of both AD and, as now shown, FTD; moreover, we have developed agents and defined new approaches to block this hyperactive pathway, and they may well be therapeutically useful.

### Introduction

In exploring underlying mechanisms for AD risk in O/MetS/T2D, we found that hyperinsulinemia in diet-induced and genetic forms of O/MetS/T2D in mice and monkeys provokes excessive increases in activity of brain PKC-λ/ι that activate nuclear factor κ-B (NFκB), which, in turn, increases expression of both β-amyloid precursor protein cleaving enzyme-1 (BACE1) and proinflammatory cytokines (CKs), tumor necrosis factor-α (TNF-α) interleukin-1β (IL-1β and IL-6 (9-11). In addition to increasing BACE1-dependent Aβ-peptides that form AD plaques, PKC-λ/ι hyperactivity increases levels of phospho-thr-231-tau (p-tau) (9-11), which may impair neuronal function and abet AD tangle formation. Moreover, prelim findings show that plaque development, tau phosphorylation and memory losses in transgenic (Tg) APP/PS1 AD mice are abrogated by both chemical inhibition and knockout (KO)-induced deficiency of brain PKC-λ/ι. Further note: (a) PKC-λ/ι is activated not only by insulin and various neurotropic factors that activate phosphatidylinositol 3-kinase (PI3K) (12-16), but also by Aβ-peptides (17), inflammatory factors (18-20), and elevations of carbohydrates and lipids, e.g., ceramide, phosphatidic acid (21-24); and (b) PKC-λ/ι hyperactivity is seen in the brains of Tg APP/PS1 mice (prelim data) and humans with non-diabetic AD (25). In short, PKC-λ/ι can abet AD development, regardless of etiology.

Although a role for PKC-λ/ι in FTD has had little attention, we found in LA1-5s neural and HMC-3 microglial cells, that progranulin (PGRN), whose deficiency can cause FTD, diminishes insulin-stimulated increases in IRS-1 and PKC-λ/ι activities, and thereby blocks PKC-λ/ι-dependent increases in Inhibitor of kappa-B kinase-α/β (IKKα/β and NFκB activities, and expression of BACE1 and CKs. The importance of a relationship between PGRN and PKC-λ/ι was underscored by the fact that PGRN injection into the hippocampus of Tg 5×FAD mice corrects AD/BACE1 pathology (26). This improvement coincides with our prelim findings showing that (a) PGRN restrains insulin-stimulated PKC-1/i activity by inhibiting IRS-1 activation, and (b) there are marked improvements in Tg APP/PS1 AD mice with both inhibition and KO-induced deficiency of PKC-λ/ι. We therefore believe that PGRN improves AD by inhibiting PKC-λ/ι.

We further postulated that PGRN deficiency in FTD leads to activation of IRS-1, PKC-λ/ι, the IKK/NFκB inflammatory pathway, and FTD pathology. This idea is further supported from our recent findings that (a) PGRN-deficient mice have increased activities of IRS-1, PKC-λ/ι, IKKcα/β and NFκB in brain; and (b); KO of PGRN activity in microglial cells by CRISPR/Cas9 methods led to maximal activation of IRS-1, PKC-λ/ι and NFκB, without altering IRS-2 or Akt activities.

These findings suggesting that PGRN selectively inhibits IRS-1 and PKC-λ/ι, and PGRN deficiency selectively activates IRS-1 and PKC-λ/ι, however, did not provide an underlying mechanism. But more recently, we found in microglial cells, that KO-induced PGRN deficiency diminishes, and PGRN treatment increases, the phosphorylation of serine-307 in IRS-1. Note that ser-307 phosphorylation is a well-known inhibitor of IRS-1 and, very interestingly, is provoked by c-Jun N-terminal kinase (JNK), which is activated by stress-release factors, such as TNF and IKK (66). Whether other inhibitory serine phosphorylations are involved remains to be determined, but we found that ser-616 and ser-636 are not involved in the effects of PGRN on IRS-1. In any case, we postulate that the phosphorylation of ser-307 residues in IRS-1 is largely/fully responsible for IRS-1 regulation by PGRN.

And, very importantly, in PGRN-KO mice, we found that the activity of IRS-1, but not IRS-2, is basally elevated body-wise, or at least in all tissues examined, including, brain, white adipose tissue, liver and skeletal muscle; and, uniquely in brain, IRS-1 activation is attended by (a) increased activities of PKC-λ/ι, IKKα/β and NFκB, but (b), in marked contrast, decreased activities of IRS-2 and Akt. Alterations in aPKC and Akt activities in fat liver and muscles of PGRN KO mice coincide with previously reported findings of tissue-specific differences in the dependence of aPKC and Akt on IRS-1 vis-a-vis IRS-2 that are discussed in greater detail below.

Accordingly, we propose: (a) PKC-λ/ι hyperactivity is a major activator of the IKK/NFκB inflammatory pathway in the brain and abets pathology development in both PGRN-deficient FTD and AD; and (b) PGRN deficiency in FTD leads to decreases in ser-307 phosphorylation and thus increases in brain IRS-1 activity that uniquely in brain selectively increases PKC-λ/ι activity, that we believe lead to the observed decreases in IRS-2 and Akt activities in PGRN-deficient mice, that would add further insult to the brain.

### Essentiality of BACE1 in AD.

In brain, BACE1 acts on DAPP to produce Aβ-peptides that form AD-plaques (27-30); indeed, BACE1 KO blocks Aβ-peptide production and plaque formation in Tg AD mice carrying mutations in APP and presenilin-1 (PS1) that increase actions of BACE1 and (3-secretase (31,32). Although brain BACE1 levels are increased in human AD (33), increases in Aβ-peptides may also reflect (a) enhanced trafficking of BACE1-containing and APP-containing vesicles to endosomes and the Trans Golgi Network, where BACE1 is activated, and (b) diminished clearance of Aβ-peptides. In any case, BACE1 is essential for AD development, and this spurred development of BACE1 inhibitors that unfortunately failed to improve memory in clinical trials. This failure of BACE1 inhibitors may reflect: (a) failure to improve tau phosphorylation and/or NFκB-dependent inflammation; (b) BACE1 requirements in memory; (c) compensatory increases in inhibited/dysfunctional forms of BACE1 that impair vesicle trafficking and memory function. In this regard, BACE1 KO does not impair memory function (31,32).

### Control of BACE1 by PKC-λ/ι

We reported that PKC-λ/ι acts via IKKcα/β and NFκB and tightly controls production of BACE1 mRNA and protein, both in brain, where BACE1 acts on βAPP to generate Aβ-peptides (11), and in liver (11,34,35), where BACE1 destroys IR-0 (34). Moreover, we developed agents that, when given orally (PO), SC or IV, inhibit liver and brain PKC-λ/ι and reduce NFκB, BACE1 and CKs, but, when given nasally, inhibit brain, but not liver, PKC-λ/ι, and reduce brain levels of BACE1, p-tau and CKs (11). Prelim findings show that treatments of Tg APP/PS1/AD mice with either PO or nasal PKC-λ/ι inhibitors prevent development of AD pathology and memory impairments.

### Hyperinsulinemia in O/MetS/T2D as a Risk Factor in AD.

As AD and T2D frequently co-exist, [80% of AD patients at Mayo Clinic have T2D or pre-T2D/MetS (1)], it appears that systemic insulin resistance is an AD risk factor. Further, as brain IR activity is partially reduced in brains of humans with non-diabetic AD (25), and, as brain IR levels in AD may be diminished by degradation of the IR-β subunit by BACE1 (34) [which is controlled by PKC-λ/ι (11), which is hyperactive in brains of humans with non-diabetic AD (25)], the resulting insulin resistance may lead to hypo-insulinization of the brain. However, the partial resistance at the IR level in brain, is in fact overcome by the hyperinsulinemia that exists in in O/MetS/T2D mice and monkeys (9,10), presumably via use of “spare” IRs, i.e., IRs present in excess of that needed for maximal activation of downstream factors. Also, in human brain, IGF-1 receptors respond to high insulin levels (25).

With the prevailing assumption that the brain is hypo-insulinized in all forms of AD, we were surprised to find (10) in brains of four mouse and one monkey model of O/MetS/T2D that insulin signaling to the IR and both Akt and aPKC, is maximally increased by the existing hyperinsulinemia (9,10). Indeed, correction of hyperinsulinemia, elicited by improvement in hepatic abnormalities by a liver-specific aPKC inhibitor (36), was attended by: (a) decreases in resting activities of brain Akt and PKC-λ/ι to normal basal levels; and (b) restored ability of insulin to maximally activate both Akt and PKC-λ/ι (10).

Of further note, both Akt and PKC-λ/ι, for unknown reason, are strongly/maximally activated in brains of humans with non-diabetic AD (25); thus, the failure of insulin to have further effect on these factors does not mean that the brain is insulin-resistant [as was frequently misconstrued.

Insulin resistance and hypoinsulinization of the brain in AD Whereas our contention that hyperinsulinemia is a risk factor for AD seems to clash with the idea that the brain is hypoinsulinized in AD (37-40), it does not contradict the idea that, in normo- or hypo-insulinemic AD, there are decreases in IR activity or levels that limit insulin action and its salutary effects. Indeed, our finding that brain IR-β levels are diminished by PKC-λ/ι-dependent BACE1 provides an underlying mechanism, and this situation may obtain in non-diabetic AD and in later insulinopenic stages of T2D-related AD. These differences in blood insulin levels in T2D may explain variable outcomes with nasal insulin treatment in AD (37-41).

### Control of BACE1 Transcription by NFkB and PKC-λ/ι

NFκB is clearly known to be a major transactivator of the BACE1 gene in mouse brain (42-44); and we have shown that NFκB is activated by PKC-λ/ι in mice and thus tightly controls BACE1 expression in both brain and cultured neural cells (11),

Activation of NFκB by aPKC.

We surprisingly found that hyperinsulinemia in O/MetS/T2D mice (21,23,36,45-47) and humans (48,49), leads in liver to: (a) IKK activation; (b) dissociation of Inhibitor of NFκB Inhibitor (IκB) from NFκB and nuclear translocation and phosphorylation/activation of the p65/RelA subunit of NFκB; and (c) increases in mRNA and protein levels of BACE1, TNF-α, IL-1β and IL-6. We further showed that insulin itself was responsible and acting via PKC-1/i, as all effects are blocked by chemical inhibitors of aPKC, expression of kinase-inactive aPKC, and PKC-λ/ι KO (11,21,23,36,45-47). Indeed, NFκB is activated within 15-min of insulin treatment, occurs with feeding, and increases CKs (11,46,47). Others have similarly found that aPKC activates NFκB during lymphocyte activation (50,51).

Tissue-Specific Activation of Akt Vis-à-Vis aPKC by IRS-1 Vis-à-Vis IRS-2.

Although insulin activates both Akt and aPKC, we found in IRS-1 KO mice that Akt activation is impaired in fat, muscle and liver, and aPKC activation is impaired in fat and muscle, but not in liver (52). We later found in liver-specific IRS-1-KO and IRS-2-KO mice that insulin activates hepatic aPKC via IRS-2, and Akt is activated mainly via IRS-1 (53); similar IRS dependencies were seen in human hepatocytes (48,49). In brain, whereas Akt activation is clearly diminished in IRS-2 KO mice (54-58), the role of brain IRS-1 has never been defined.

Tissue-Specific Activation of Akt Vis-a-Vis aPKC by IRS-1 Vis-à-Vis IRS-2 as Related to PGRN.

In concert with our IRS-I and IRS-2 KO findings (52,53), in fat and adipocytes (59), and in liver and hepatocytes (60), PGRN treatment diminishes IRS-1 and Akt activation by insulin, and acute knockdown (KD) of PGRN increases IRS-1 and Akt activation (aPKC was not studied). Similarly, we found in prelim studies that PGRN deficiency in KO mice leads to hyperactivity of IRS-1, but not IRS-2, in all tissues examined (fat, liver, muscle, brain), and IRS-1 hyperactivity leads to: (a) Akt hyperactivity in fat, liver and muscle, but not in brain, and (b) aPKC hyperactivity in fat, muscle and brain, but not in liver; our findings coincide with findings in IRS-1 and IRS-2 KO mice (52,53). Note that IKK and NFκB are activated in all tissues of PGRN KO mice wherein aPKC is activated.

Brain-Specific Activation of Akt and aPKC by IRS-1 Vis-à-Vis IRS-2 as Related to PGRN.

Prelim studies show that: (a) IRS-1 and aPKC activities are increased, and IRS-2 and Akt activities are decreased, in brains of both fed and overnight-fasted PGRN-deficient mice; (b) changes in brain are greater in homozygous than in heterozygous PGRN KO mice, and are amplified by feeding, presumably via secreted insulin; and (c) PGRN treatment diminishes insulin activation of IRS-1 and PKC-λ/ι, but not IRS-2 and Akt, in human-derived LA1-5s neural and HMC-3 microglial cells. These data are the first strongly suggesting that, in brain, IRS-2 largely regulates Akt, and IRS-1 largely regulates PKC-λ/ι.

Consequences of Alterations of Akt and aPKC Activity in PGRN-Deficient Mice.

Collectively, past and present findings suggest that PGRN inhibits basal IRS-1 throughout the body, and PGRN deficiency reverses this inhibition and enhances the activation of IRS-1 and thus, Akt, or aPKC, or both, depending upon the cell-type. Thus, PGRN deficiency and IRS-1 hyperactivity hyperactivate (a) both Akt and aPKC in fat and muscle, and thus glucose transport, which requires both kinases (61); and (b) only Akt in liver, which suppresses gluconeogenesis (61). These findings may explain why PGRN-deficient mice are insulin-sensitive and protected against developing obesity and T2D when fed a HFD (59,60,62). In contrast, in brain, IRS-1 hyperactivity leads to hyperactivation of PKC-λ/ι, IKK α/β, and NFκB, and NFκB-dependent increases in BACE1 may explain the decreases in IR levels and activities of IRS-2 and Akt seen in brains of PGRN-deficient mice (prelim data). These decreases in activity of Akt in brain may: impair LTP and cerebral functions (55); increase FoxO1 activity and promote apoptosis (63) and diminish mTOR/S6K activity and impair protein synthesis and increase autophagy and lysosomal activity (64,65).

### Mechanism for IRS-1 Activation by PGRN Deficiency.

Insulin, IGF-1 and certain other tyrosine kinases (TKs) activate IRS-1 and IRS-2 by phosphorylating tyrosine (tyr, Y) residues; oppositely, IRS-1 is inhibited by phosphorylation of various serine (ser), or less so, threonine (thr) residues, as elicited by mTOR, S6K, ERK1/2, JNK1/2/3 and other factors (66) (in our experience, IRS-2 is less vulnerable to inhibition). Most importantly, phosphorylation of ser-307 in IRS-1 is diminished in PGRN KO (vs WT) cells and in tissues of PGRN KO mice, and, moreover, PGRN dose-relatedly increased ser-307 phosphorylation, but not that of ser-616 or ser-636 in PGRN KO cells (prelim data). Further note, ser-307 phosphorylation in IRS-1 is most conspicuously increased by the activation of JNK (66) which is activated by stress-response-related factors, such as CKs (66). Thus, we postulate that, normally, throughout the body, JNK limits basal IRS-1 activity by phosphorylating ser-307 [and as per initial indications, ser-302, which is also JNK-dependent (102)], and, with PGRN deficiency, this basal effect of PGRN on phospho-307/302 is lost and IRS-1 is basally/constitutively activated. In this regard, PGRN is postulated to inhibit IRS-1-dependent insulin signaling by increasing TNF in hepatocytes (60,62) and IL-6 in adipocytes (59), and these stress factors could contribute to normal basal activation of JNK (66) and inhibition of IRS-1 [see (103)] by PGRN.

In any case, it is uncertain how increases in JNK activity, and subsequent increases in IRS-1 307 phosphorylation and activities of IRS-1, PKC-λ/ι and NFκB, may (or may not) relate to lysosomal aberrations prominently seen in brains of humans with PGRN-deficient FTD (80,64,65). On the one hand, IRS-1/PKC-λ/ι-dependent NFκB hyperactivity may cause inflammation and tissue damage that activates lysosomes; additionally, decreases in brain IRS-2/Akt activity may (a) diminish mTOR activity and thus increase autophagy (66) and lysosomal activity (64), and (b) increase FoxO activities and thus enhance apoptosis (63). On the other hand, PGRN deficiency-induced effects in lysosomes that are IRS-1-independent may release stress factors that activate JNK and increase ser-307-IRS-1 phosphorylation.

aPKC Inhibitors. We identified by HTS compounds that target the substrate-binding site of aPKCs and potently (IC50s10-30 nM) inhibit recombinant PIP₃-dependent aPKCs, but not DAG-dependent PKCs. Three compounds have been studied in detail: (a) 1H-imidazole-4-carboxamide, 5-amino-1-[2,3-dihydroxy-4-[(phosphono-oxy)methyl]cyclopentyl-[1R-(1a,2b,3b,4a)] (ICAPP), which inhibits PKC-λ/ι (IC50,1-10 nM); (b) 1H-imidazole-4-carboxamide, 5-amino-1-[2,3-dihydroxy-4-[(hydroxyl)methyl] cyclopentyl-[1R-(1a,2b, 3b,4a)] (ICAP), which is converted to active phosphorylated ICAPP intracellularly by adenosine kinase (81); and (c) 2-acetyl-cyclopentane-1,3-diketone (ACPD). In doses just sufficient to inhibit hepatic aPKC and reverse T2D-induced aberrations, these inhibitors do not inhibit brain, muscle or adipocyte aPKC, and have no effect on Akt, AMPK, or an array of 35 kinases independently tested (21,22,36,48,49,81,82). However, at higher doses, ICAPP, ICAP and ACPD inhibit brain aPKC (9-11). Thus, these agents can reduce brain aPKC activity, indirectly at lower doses by inhibiting liver aPKC and correcting hyperinsulinemia in insulin-resistant states, and, at higher doses, by acting directly on brain PKC-λ/ι. Note that published (83) and prelim PK/PD studies show that ICAP preferentially distributes to liver, but clearly passes the BBB and enters the brain. Moreover, ICAP is effective in brain when given per os (PO) and nasally, as well SC (11). In addition to ICAP and ACPD, gold-containing aurothiomalate (ATM) and auranofin (AF) [(2,3,4,6-tetra-O-acetyl-1-thio-3-D-glucopyranosato-S-)(triethyl-phosphine)-gold)] inhibit insulin activation of aPKC by inhibiting the PB1 site (84) that dimerizes with other PB1 sites, most notably, scaffolding protein p62 (85-87) (aka, sequestosome-1), a cargo protein for ubiquinated proteins. Note that AF is FDA-approved for rheumatoid arthritis, is orally effective, and PO AF is known to cross the BBB and protect against development of neuroinflammation (88).

aPKC Requirements for Memory Function.

Various findings suggest that brain 50 kDa PKC-ζ (aka, “PKMζ”) which is constitutively active, functions in long-term potentiation (LTP) and memory (LTM) (89,90). Much differently, from (a) lack of effect of PKC-λ/ι inhibitor ICAP on LTP and LTM (90), (b) normal standard memory testing and LTP in neuron-selective PKC-λ KO mice (91), and (c) normal memory testing in PKC-λ/ι haploinsufficient mice (11), a full complement of PKC-λ/ι is not required for LTP/LTM. Also note that aPKC inhibitor ACPD improves memory function in HFF mice (9).

### Knockout (KO) of PKC-λ/ι in Brains of Tg APP/PS1 AP Mice Corrects AD-Like Aberrations

In AD studies, we generated mice with total body heterozygous (Het) KO of PKC-λ/ι and mice with

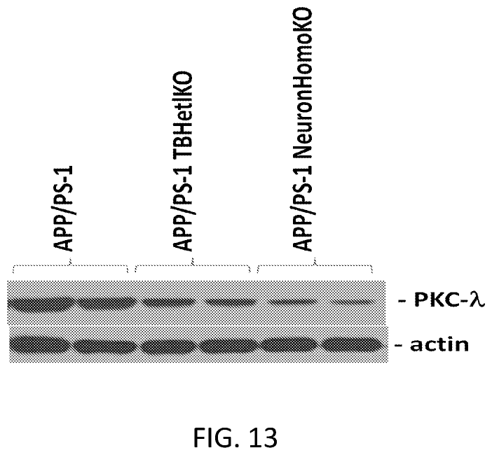

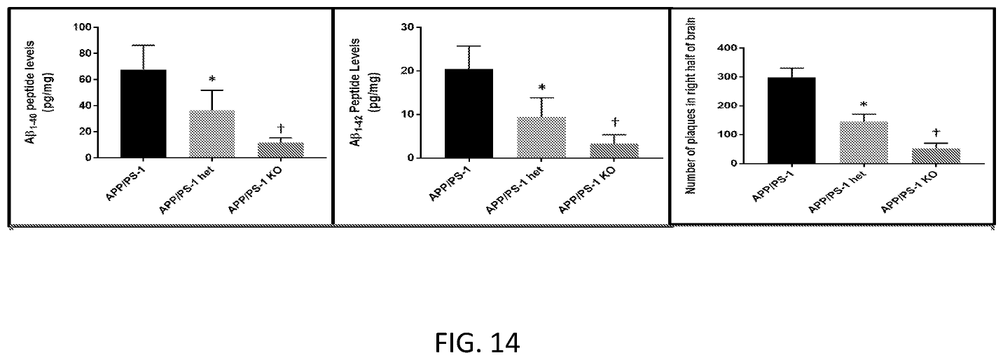

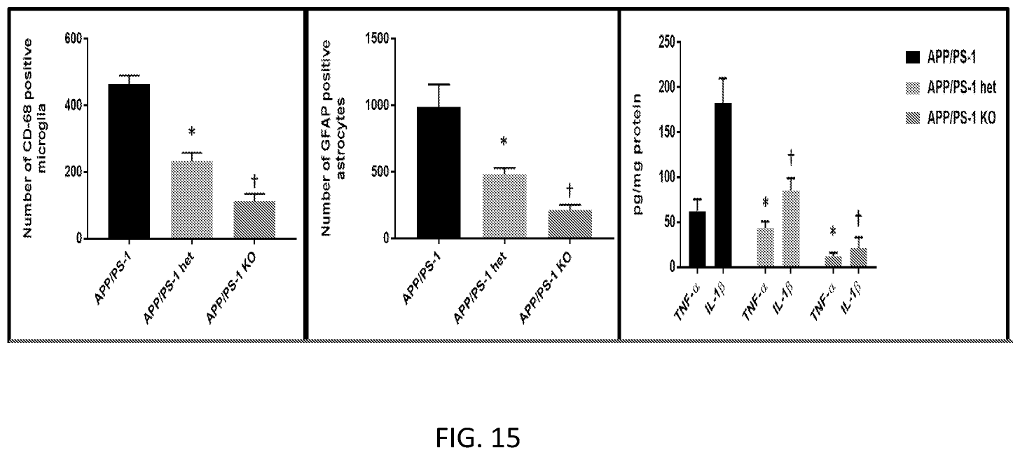

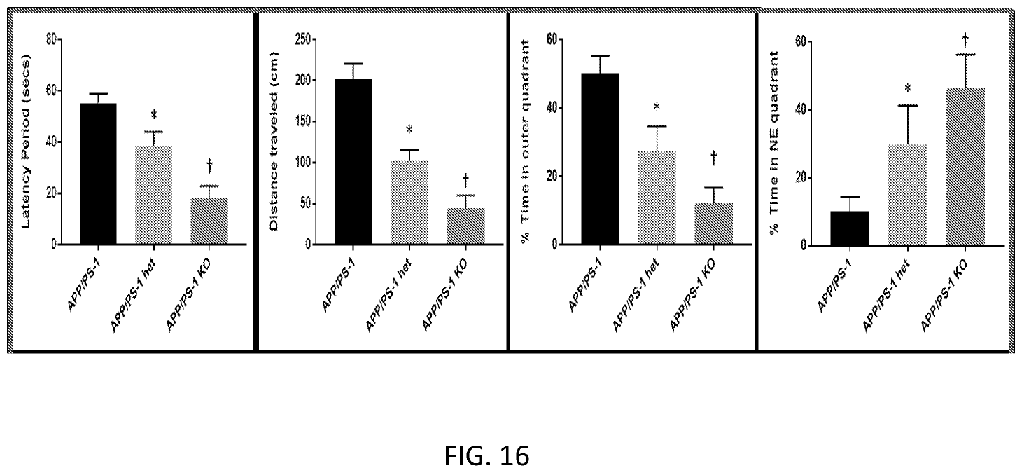

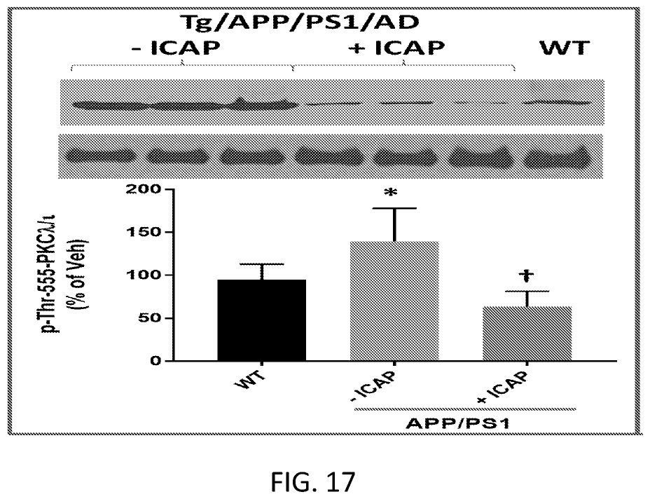

conditional, neuron-specific homozygous (Homo) KO of PKC-λ, and then crossed both KO mice with Tg APP/PS1 AD mice to generate AD mice without (WT) or with deficiency of PKC-λ throughout the body (TBHetKO; “Het”) or specifically in neurons (Neuron-Homo-KO, “KO”) (FIG. 13). Surprisingly, we found (FIG. 17) that PKC-λ is hyperactivated in brains of Tg/APP/PS1/AD mice, perhaps via increases in Aβ-peptides (17) or hyperinsulinemia, owing to development of glucose intolerance in Tg/APP/PS1/AD mice, as seen by us and others. Most importantly, in TBHetKO and Neuron-HomoKO mice, stepwise decreases in brain PKC levels (FIG. 13) led, in Tg AD mice to stepwise: (a) decreases in Aβ-peptides and Aβ-plaque levels (FIG. 14); (b) decreases in CK levels (FIG. 15); (c) decreases in inflammatory responses in microglial (CD-68 staining) and astroglial (GFAP staining) cells (FIG. 15); and (d) improvements in learning/memory function in the radial arm water maze (RAWM) test (FIG. 16). In accord our recent report of marked decreases of BACE1 levels in mice with PKC-λ haploinsufficiency (11), brain BACE1 levels were diminished in both KO mice (not shown here; but see BACE1 decreases in ICAP-RX studies).

### ICAP Rx Corrects Hyperactivity of Brain PKC-λ/ι and AD Pathology in Tg/APP/PS1/AD Mice

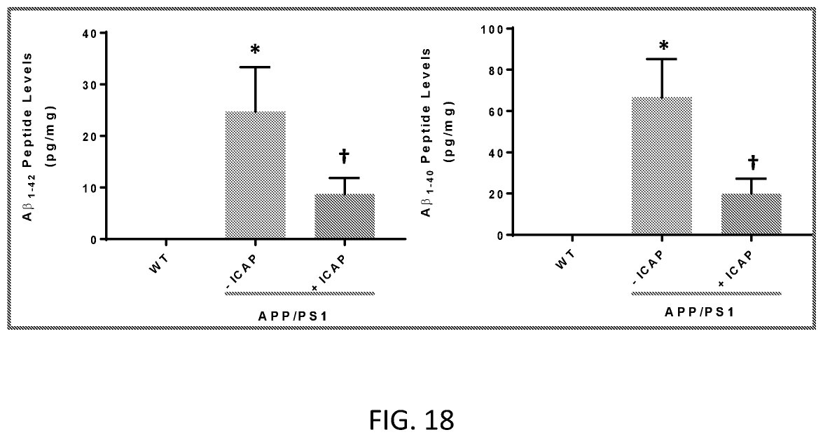

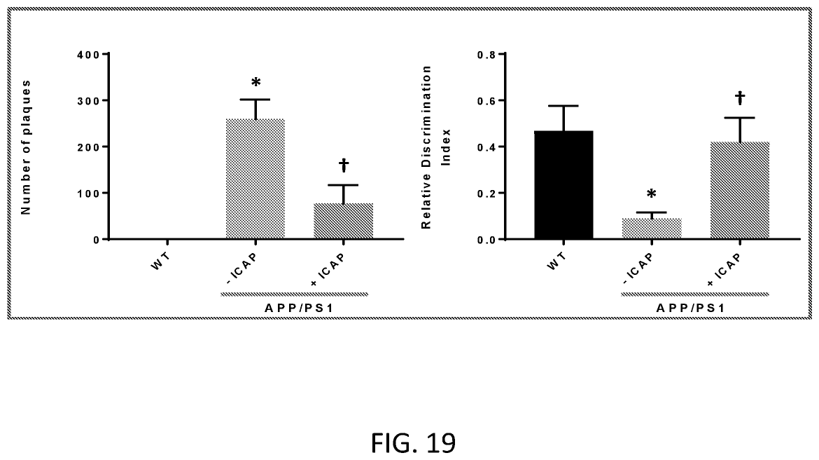

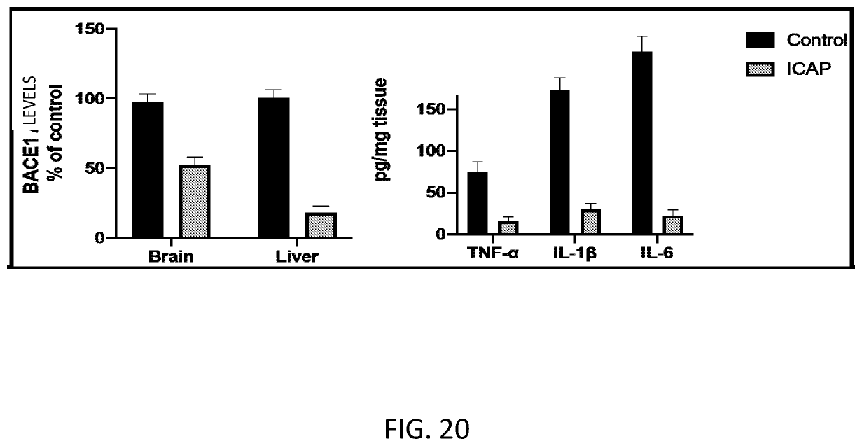

As alluded to, resting PKC-λ/ι activity was increased by approx. 40% in brains of Tg APP/PS1 AD mice, relative to normal WT mice (FIG. 17). We treated 3-month-old APP/PS1/AD mice for the next 3 months with PO ICAP (given daily by gastric lavage), and PO ICAP reduced PKC-λ/ι activity (FIG. 17), and simultaneously reduced Aβ40 and Aβ42 levels (FIG. 18) and Aβ-plaque load by approx. 70% (FIG. 19, left). Moreover, PO ICAP: (a) restored memory functions in both Novel Object Recognition (NOR) (FIG. 19, right) and RAWM (not shown, but see above) tests; and (b) diminished levels of BACE1 and CKs (TNF-α, IL-1β, IL-6) to normal (FIG. 20) that were markedly elevated in brains of Tg APP/PS1 AD mice. Like PO ICAP, nasal ICAP treatment (which inhibits brain, but not liver, aPKC) corrected AD pathological and functional aberrations in APP/PS1 AD mice (not shown).

PGRN Inhibits Activation of IRS-1, PKC-λ/ι, IKK α/β and NFκB, butnotAkt, in Neural and Glial Cells

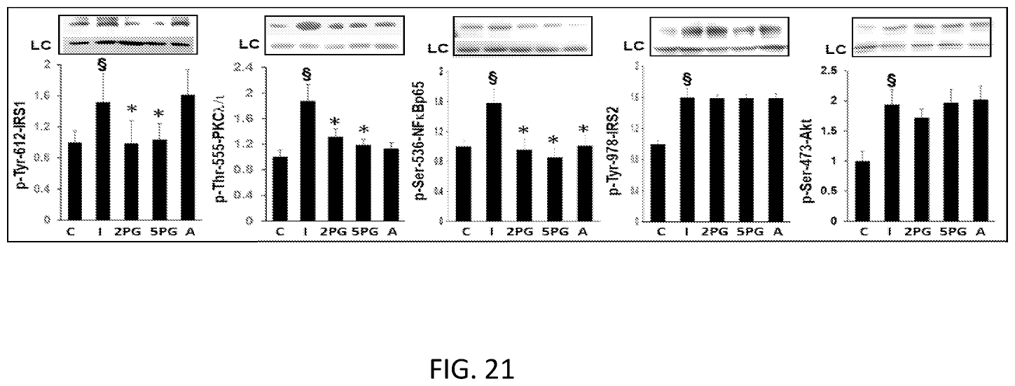

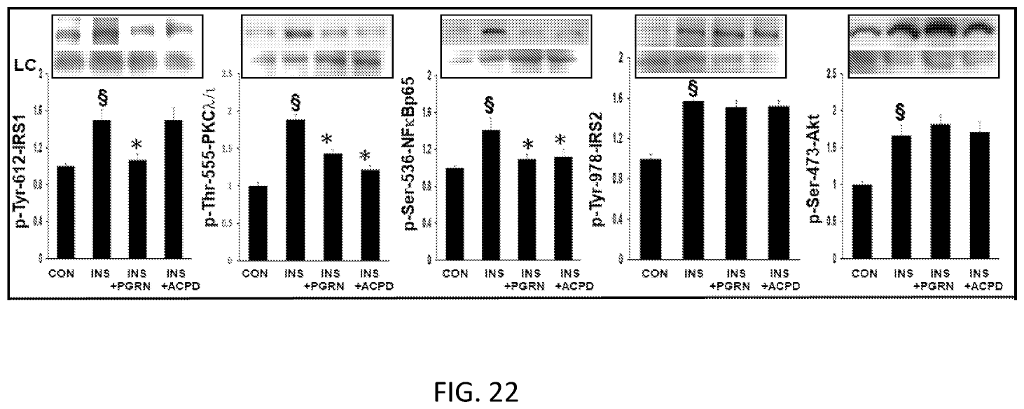

We recently reported that insulin activates PKC-λ/ι and thereby activates IKK α/β and NFκB and increases BACE1 expression in human-derived LA1-5s neural cells (11). Here, we found that insulin activation of IRS-1, PKC-λ/ι, IKK α/β and NFκB, but not IRS-2 and Akt, was inhibited by 200-500 ng/ml PGRN [all protein levels were unaltered by treatments]; and, as expected, aPKC inhibitor, ACPD, inhibited IKK and NFκB, but not IRS-1, IRS-2 or Akt, activation (FIG. 21); comparable results were seen in HMC-3 microglial cells (FIG. 22). Thus, as we reported in liver and hepatocytes, insulin activates NFκB via PKC-λ/ι and increases expression of BACE1 and CKs in neural and glial cells; however, unlike liver where IRS-2 activates PKC-λ/ι (52,53), PKC-λ/ι is activated by IRS-1 in brain; similar correlation between IRS-2 and PKC-1/i activation in non-CNS tissues (liver, muscle, fat) were seen in PGRN-deficient mice (see below).

### IRS-1-Dependent Insulin Signaling is Increased in PGRN-Deficient Mice.

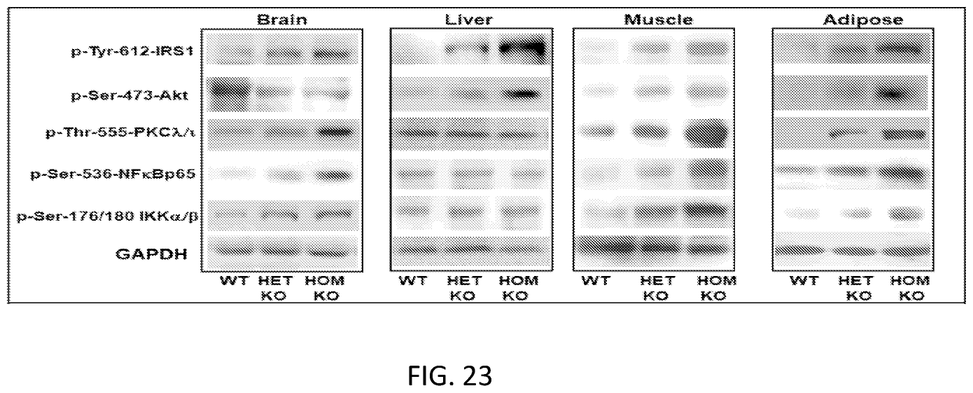

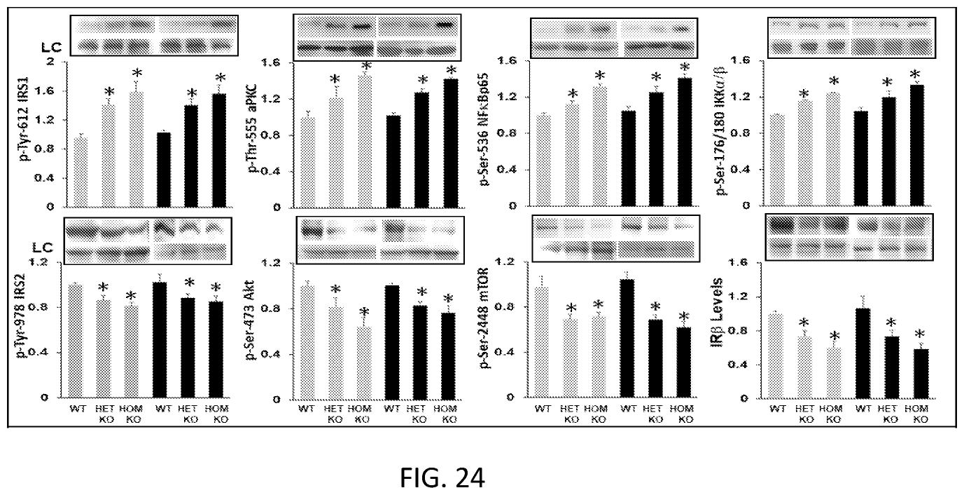

In 2-6-month-old overnight-fasted and ad lib fed PGRN^{+/-} and PGRN^{-/-} mice: (a) activities of IRS-1, but not IRS-2, were elevated in all tissues examined (brain, fat muscle, liver); (b) activities of IRS-1, PKC-λ/ι, IKK α/β and NFκB, but not IRS-2 and Akt, were elevated in brain; (b) activities of IRS-1, Akt, PKC-λ/ι, IKK α/β and NFκB were elevated in fat and muscle; and (c) activities of IRS-1 and Akt, but not PKC-λ/ι, IKK μ/β or NFκB, were elevated in liver (FIG. 23). These correlations between IRS-1 vis-a-vis IRS-2 and Akt vis-à-vis aPKC in fat, liver and muscle coincide with previous findings in IRS-1 and IRS-2 KO mice [discussed above and see (52,53)]. More extensive studies of brain are depicted in FIG. 24 and showed that: (a) IRS-1, PKC-λ/ι, IKK α/β and NFκB activities increased stepwise with increasing PGRN deficiency; (b) IRS-2, Akt and mTOR activities decreased stepwise with increasing PGRN deficiency, perhaps as a result of the loss of PGRN effects on IRS-2 and Akt, and/or decreases in IR-β levels (FIG. 24) that were most likely caused by PKC-dependent increases in brain BACE1 levels. Although not shown in FIG. 24, activities of all signaling factors were approx. 20-30% higher in fed mice vis-à-vis fasted mice; thus, whereas basal increases in PKC activity owing to PGRN-deficiency-induced IRS-1 inhibition in fasted mice may or may not require at least some insulin, PKC-λ/ι and other signaling factors can be further activated by increases in the insulin/IR/IRS-1 pathway.

aPKC Increases BACE1 and Decreases IR-β Levels in Mouse Brain

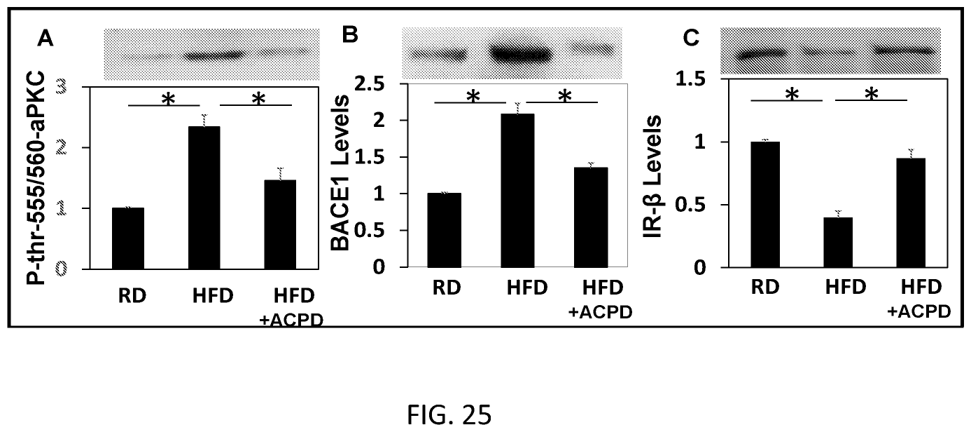

BACE1 reportedly degrades IR-0 and partially diminishes functional IR levels in livers of obese/T2D mice (34). We similarly found in both liver and brain [(35) and FIG. 25)] that HFD/hyperinsulinemia-induced increases in PKC-λ/ι activity and BACE1 levels lead to decreases in IR-β levels; and, most importantly, all changes are reversed by ACPD treatment (FIG. 25). Thus, PKC-λ/ι hyperactivity in brains of PGRN-deficient mice may help to explain decreases in IR-β levels and deficient activities of IRS-2/Akt/mTOR (FIG. 25).

### REFERENCES

1. Janson J, Laedtke T, Parisi J E, O'Brien P, Peterson R C, Butler P C. Increased risk of type 2 diabetes in Alzheimer's disease. Diabetes 53: 478-481, 2004
2. Ott A, Stolk R P, Hofman A, van Harskamp F, Grobbee D E, Breteler M M B. Association of diabetes mellitus and dementia: the Rotterdam Study. Diabetologia 1996; 39:1392-1397
3. Profenno, L. A., Anton P., Porsteinsson, S., Faraone. V. Meta-Analysis of Alzheimer's Disease Risk with Obesity, Diabetes, and Related Disorders. Biological Psychiatry 67: 505-512, 2010,
4. Cukierman T, Gerstein H C, Williamson J D. Cognitive decline and dementia in diabetes-systematic overview of prospective observational studies. Diabetologia 2005; 48: 2460-2469
5. Cheng C-K, Tsau Y-C, Su Y-C, Sung P-C, Tui H-C, Kung W-M. Metabolic risk factors of Alzheimer's disease, dementia with Lewy bodies, and normal elderly: a population-based study. Behavioral Neurology 2018, Article ID 8312346
6. Sandyk R. The relationship between diabetes mellitus and Parkinson's disease. Int J Neurosci 69: 125-130, 1993.
7. Golimstok A, Cámpora N, Rojas J, et al. Cardiovascular risk factors and frontotemporal dementia: a case-control study. Translational Neurodegeneration 3:13, 2014.
8. Ahmed R M, MacMillan M, Bartley L, Halliday G M, Kiernan M C, Hodges J R, MD Olivier Piguet O. Systemic metabolism in frontotemporal dementia. Neurology 83, 2014
9. Sajan M P, Hansen B C, Ivey R A, III, et al. Brain insulin signaling is increased in insulin-resistant states and decreases in FoxOs and PGC-1 and increases in A 1-40/42 and phospho-tau may abet Alzheimer's development. Diabetes 65; 1892-1903, 2016.
10. Sajan M P, Ivey R A, III, Hansen B C, et al., Atypical PKC, PKC activates β-secretase and increases Aβ_{1-40/42} and phospho-tau in mouse brain and isolated neuronal cells, and may link hyperinsulinemia and other aPKC activators to development of pathological and memory abnormalities in Alzheimer's Disease. Neurobiol Aging 43: 62-73, 2018. 11.
11. Sajan M P, Leitges M, Park C, Diamond D M, Wu J, Hansen B C, Duncan M A, Apostolakis C A, Apostolakis A M, Kindy M, Farese R V. Control of b-site amyloid precursor protein cleaving enzyme-1 expression by protein kinase C-l/i and nuclear factor k-B. Curr Alz Res 18: 941-955, 2021
12. Miranda C, Miele G A, Pierotti M A, Van Obberghen E. IRS-1 and IRS-2 are recruited by TrkA receptor and oncogenic TRK-T1. J Cell Physiol 186: 35-46, 2001
13. Woolen M W, Selbenhener M L, Neidigh K B, Vandanplas M L. Mapping of atypical protein kinase C within the nerve growth factor signaling cascade: relationship to differentiation and survival of PC12 cells. Mol Cell Biol 20:4494-4504, 2000
14. Melemedjian O K, Tillu D V, Asiedu M N, et al. BDNF regulates atypical PKC at spinal synapses to initiate and maintain centralized chronic pain state. Molec Pain 9: 12-26, 2013
15. Brennan-Minnella A M, Shen Y, Swanson R A. PI3K couples NMDA receptors to superoxide release in excitotoxic neuronal death. Cell Death Disease 4, e580; doi; 10.1038, 2013
16. Koponen S, Kurkinen K, Akerman K E, et al. Prevention of NMDA-induced death of cortical neurons by inhibition of protein kinase Czeta. J Neurochem 86: 442-450, 2003.
17. Ciccocioppo F, Lanuti P, Marchisio M, et al. Expression and phosphorylation of protein kinase isoforms in Aβ₁₋₄₂ activated T lymphocytes from Alzheimer disease. Intematl J Immunopathol and Pharmacol 21:23-33, 2008;
18. Anthonsen M W, Andersen S, Solhaug A, Johansen B. aPKC Conveys 5-lipoxygenase leucotriene B4-mediated cross-talk between phospholiase A2s regulating NFκB activation in response to tumor necrosis factor-α and interleukin-1β. J Biol Chem 276: 35344-35351, 2001
19. Thaler J P, Choi S J, Sajan M P, Farese R V. et al. Atypical protein kinase C activity in the hypothalamus is required for LPS-mediated sickness responses. Endocrinol 150: 5362-5372, 2009
20. Takada Y, Mukhopadhyay A, Kundu G C, Mahabeleshwar G H, Singh S, Aggarwal B B. Hydrogen Peroxide Activates NF-kappa B Through Tyrosine Phosphorylation of I Kappa B Alpha and Serine Phosphorylation of p65: Evidence for the Involvement of I Kappa B Alpha Kinase and Syk Protein-Tyrosine Kinase J Biol Chem 278: 24233-24241, 2003
21. Sajan M P, Acevedo-Duncan M E, Standaert M L, et al. Akt-dependent phosphorylation of hepatic FoxO1 is compartmentalized on a WD40/Propeller/FYVE scaffold and is selectively inhibited atypical PKC in early phases of diet-induced obesity. A mechanism for impairing gluconeogenic but not lipogenic enzyme expression. Diabetes 63: 2690-2701, 2014
22. Sajan M P, Ivey R A, III, Lee M C, Farese R V. Hepatic insulin resistance in ob/ob mice involves increases in ceramide, atypical PKC activity and selective impairment of Akt-dependent Fox01 phosphorylation. J Lipid Res 56: 70-80, 2015.
23. Sajan, M. P., Bandyopadhyay, G., Kanoh, Y., et al. Sorbitol activates atypical protein kinase C and GLUT4 glucose transporter translocation/glucose transport through proline-rich tyrosine kinase-2, the ERK pathway and PLD. Biochem. J. 362: 665-674, 2002
24. Bandyopadhyay G, Sajan M P, Kanoh Y, et al. Glucose activates protein kinase C-Q/X through proline-rich tyrosine kinase-2, ERK, and PLD J Biol Chem 276: 35537-35545, 2001
25. Talbot K, Wang Y, Kazi H, et al. Demonstrared brain insulin resistance, IRS-1 dysregulation, and cognitive decline. J Clin Invest 122:1316-1338, 2012
26. Guan Z, Chen Z, Fu S, Dai L, Shen Y. Progranulin administration attenuates b-amyloid deposition in the hippocampus of 5×FAD mice through modulating BACE1 expression and microglial phagocytosis. Front Cellular Neurosci 14: Article 260, 2020
27, Sinha S., Lieberburg, I. Cellular mechanisms of 0-amyloid production and secretion. Proc. Natl. Acad. Sci, USA 96, 11049-111053, 1999
28. Nunan, J., Small, D. H. Regulation of APP cleavage by α-, β- and γ-secretases. FEBS Lett. 483, 6-10, 2000
29. Cole S I, Vassar R. The Alzheimer's disease enzyme, BACE1. Molec Degen 2:22-70, 2007
30. Kandalepas P C and Vassar R. The normal and pathological roles of the Alzheimer's □-secretase, BACE1. Curr Alzheimer Res 11: 441-449, 2014
31. Hu X, Das B, Hou H, He W, Yan R. (2018) BACE1 deletion in the adult mouse reverses preformed amyloid deposition and improves cognitive functions. J Exp Med 215: 927-941
32. Kimura R, Devi L, Ohno M. Partial reduction of BACE1 improves synaptic plasticity, recent and remote memories in Alzheimer transgenic mice. J Neurochem 113, 248-261, 2010
33. Yang, L-B., Lindhol, K., Yan, et al. Elevated D-secretase expression and enzymatic activity detected in sporadic Alzheimer's disease. Nat Med 9, 3-4, 2003.
34. Meakin P J, Mezzapesa A, Benabou E, et al. The beta secretase BACE1 regulates the expression of insulin receptor in the liver. Nat Commun 9:1306, 2018.
35. Sajan M P, Braun U, Leitges M, Park C, Diamond D M, Wu J, Hansen B C, Acevedo-Duncan M, Apostopolos C A, Apostopolos A H, Kindy M A, Farese R V. Atypical PKC controls β-secretase expression and thereby regulates production of Alzheimer plaque precursor Aβ in brain and insulin receptor degradation in liver. Metabolism 104, Supplement 154112, Mar. 1, 2020
36. Sajan M P, Nimal S, Mastorides S, et al. Correction of Metabolic Abnormalities in a Rodent Model of Obesity, Metabolic Syndrome and Type 2 Diabetes by Inhibitors of Hepatic Protein Kinase C-iota. Metabolism 61:459-469, 2012.
37. Craft S. Insulin resistance and Alzheimer's disease pathogenesis: mechanisms and implications for treatment. Curr Alzheimer Res 4: 147-152, 2007
38. Craft S, Claxton A, Baker L D, et al., Effects of Regular and Long-Acting Insulin on Cognition and Alzheimer's Disease Biomarkers: A Pilot Clinical Trial. J Alzheimer's Disease. 57: 1325-1334, 2017. PMC5409050
39. Holscher C. First clinical data of the neuroprotective effects of nasal insulin application in patients with Alzheimer's disease. Alzheimers Dement. 10: 33-7. 2014.
40. Benedict, C., Grillo, C. A. (2018) Insulin resistance as a therapeutic target in the treatment of Alzheimer's disease; A state-of-the-art review. Front Neurosci 12, 215-228
41. Craft S, Raman R, Chow T W, et al. Safety, efficacy, and feasibility of intranasal insulin for treatment of mild cognitive impairment and Alzheimer dementia. JAMA 77: 1099, 2020.
42. Buggia-Prevot V, Sevalle J, RossnerS, Checler F. NFκB-dependent control of BACE1 promoter transactivation by Ab42. J Biol Chem 283: 10037-10047, 2008
43. Chen C-H, Zhou W, Liu S, Deng Y, Cai F, Tone M, Tone Y, Song W. Increased NF-κ-B signaling up-regulates BACE1 expression and its therapeutic potential in Alzheimer's disease. Internatl J Neuropsychopharmacol 15: 77-90. 2012
44. All-trans-retinoic acid reduces BACE1 expression under inflammatory conditions via modulation of nuclear factor kB (NFκB) signaling. J Biol Chem 290: 22532-22542, 2015
45. Sajan, M. P., Standaert, M. L., Nimal, S., Varanasi, U., Pastoor, T., Mastorides, S., Braun, U., Leitges, M., and Farese, R. V. The Critical Role of Atypical Protein Kinase C in Activating Hepatic SREBP-1c and NFκB in Obesity. J Lipid Research 50: 1133-1145, 2009
46. Sajan M P, Standaert M L, Rivas J, et al. Role of atypical protein kinase C in activation of sterol regulatory element binding protein-1c and nuclear factor kappa B (NFκB) in liver of rodents used as model of diabetes, and relationships to hyperlipidaemia and insulin resistance. Diabetologia 52: 1197-1207, 2009
47. Sajan M P, Ivey R A, Lee M C, et al. PKC□ haploinsufficiency prevents diabetes by a mechanism involving alterations in hepatic enzymes. Molec Endocrinol 28: 1097, 2014
48. Sajan M P, Ivey R A, III, Farese R V. BMI-Related Progression of Atypical PKC-dependent Aberrations in Insulin Signaling Through IRS-1, Akt, FoxO1 and PGC-1 in Livers of Obese and Type 2 Diabetic Humans. Metabolism 64:1454-1465, 2015;
49. Sajan M P and Farese R V. Insulin Signalling in Hepatocytes of Type 2 Diabetic Humans. Excessive Expression and Activity of PKC-λ/ι and Dependent Processes and Reversal by PKC-λ/ι Inhibitors. Diabetologia. 55:1446-1457, 2012.
50. Diaz-Meco M T, Moscat J. The atypical PKCs in inflammation: NF-κB and beyond. Immunol Rev. 2012 246: 154-167, 2012
51. Forteza R, Figueroa Y, Mashulkova A, et al. Conditional knockout of polarity complex (atypical) PKC□ reveals an antiflamatory function mediated by NFxB. Mol Biol Cell 27: 2186-2197, 2016
52. Sajan M P, Standaert M L, Miura A, Kahn C R, and Farese R V. Tissue-specific differences in activation of atypical protein kinase C and protein kinase B in muscle, liver, and adipocytes of insulin receptor substrate-1 knockout mice. Mol Endocrinol 18: 2513-2521, 2004
53. Guo S, Copps K D, Park S, Cheng Z, Pocai A, Rossetti L, Sajan M S, Farese R V, White M F. The Irs1 branch of the insulin signaling cascade plays a dominant role in hepatic nutrient homeostasis. Mol Cell Biol 29: 5070-5083, 2009
54. Costello D A, Claret M, Al-Qaassab H, Platner F, Irvine E E, Choudhury A I, Giese K P, Withers D J, Pedarzani P. Brain depletion of insulin receptor substrate 2 disrupts hippocampal synaptic plasticity and metaplasticity. PLoS ONE 7: e31124
55. Lin X, Taguichi A, Park S, Kushner J A, Li F, Li Y, White M F. Dysregulation of insulin receptor substrate 2 in b cells and brain causes obesity and diabetes. J Clin Invest 114: 908-916, 2004
56. Schubert M, Brazil D P, Burks D J, Kushner J A, Ye J, Flint C L, Farhang-Fallah J, Dikkes P, Warot X M, Rio C, Corfas G, White M F. Inulin receptor substrate-2 deficiency impairs brain growth and promotes tau phosphorylation. J Neurosci 23: 7084-7092, 2003.
57. Pardini A W, Nguyen H T, Figlewicz D P, Baskin D G, Williams D I, Kim F, Schwartz M W. Distribution of insulin receptor substrate-2 in brain areas involved in energy homeostasis. Brain Res 1112: 169-178, 2006
58. Martin E D, Sanchez-Perez A, Trejo J L, Martin-Aldana J A, Jaimez M C, Pons S, Umanzor C A, Menes L, White M F, Burks D J. IRS-2 impairs NMDA receptor-dependent long-term potentiation. Cerebral Cortex 22:1717-1727, 2012
59. Matsubara T, Mita A, Minami K, et al. PGRN is a key adipokine mediating high fat diet-induced insulin resistance and obesity through IL-6 in adipose tissue. Cell Metab 27: 38-50, 2012
60. Liu J, Li H, Zhou B, Xu L, Kang X, Yang W, Wu S, Sun H. PGRN induces impaired insulin sensitivity and defective autophagy in hepatic insulin resistance. Mol Endocrinol 29: 528-541, 2015
61. Sajan M P, BC Hansen, Acevedo-Duncan M, Kindy M S, Cooper D R, Farese R V. Roles of hepatic atypical protein kinase C hyperactivity and hyperinsulinemia in insulin-resistant forms of obesity and type 2 diabetes mellitus. MedComm https://doi.org/10.1002/mco2.54, 2021
62. Li H, Zhou B, Xu L, Liu J, Zang W, Wu S, Sun H. Circulating PGRN is significantly associated with systemic insulin sensitivity and autophagic activity in metabolic syndrome. Endocrinol 155: 3493-3507, 2014
63. Zhang, X., Tang, N., Hadden, T. J., Rishi, A. K. Akt, FoxO and regulation of apoptosis. Biochimica et Biophysica Acta 1813: 1978-1986, 201
64. Finkbeiner, S. The autophagy lysosomal pathway and neurodegeneration. Cold Spring Harb Perspect Biol PMC 2020 Oct. 1
65. Norwitz N G and Querfurth H. mTOR Mysteries: Nuances and questions about the mechanistic target of rapamycin in neurodegeneration. Frontiers Neurosci 14: 775. 2020. doi: 10.3389/fnins.2020o.00775
66. Boura-Halfon S and Zick Y. Phosphorylation of IRS proteins, insulin action, and insulin resistance. Am J Physiol Endocrinol and Metab 296: E581-591, 2009
67. Feng T, Zheng L, Liu F, et al. Growth factor progranulin promotes tumorigenesis of cervical cancer via PI3K/Akt/mTOR signaling pathway Oncotarget 7: 58381-58395, 2016
68. Hu S Y, Tai C C, Li Y H, Wu J L. Progranulin compensates for blocked IGF-1 signaling to promote myotube hypertrophy in C2C12 mvoblasts via the PI3K/Akt/mTOR pathway. FEBS Lett. 2012 Sep. 21; 586(19):3485-92. doi: 10.1016/j.febslet.2012.07.077. Epub 2012 Aug. 10. PMTD: 22967900.
69. Arechavaleta-Velasco F, Perez-Juarez C E, Gerton G L, Diaz-Cueto L. Progranulin and its biological effects in cancer. Med Oncol. 2017 Nov. 7; 34(12):194. doi: 10.1007/s12032-017-1054-7. PMID: 29116422; PMCID: PMC5810362.
70. Xu S-Q, Tang D, Chamberlain S, Pronk G, Masiarz F R, Kaur S, Prisco M, Zanocco-Marani T, Baserga R. The Granulin/Epithelin Precursor Abrogates the Requirement for the Insulin-like Growth Factor 1 Receptor for Growth in Vitro. J Biol Chem 273: 20078-20083, 1998
71. Tremblay F Krebs M, Dombrowski L, Brehm A, Bermroider E, Roth E, Nowotny P, Waldhlausl W, Marette A, Roden M. Overactivation of S6 kinase 1 as a cause of human insulin resistance during increased amino acid availability. Diabetes. 54: 2674-2684, 2005.
72. Wang L, Chen J, Hu Y, Liao A, Zheng W, Wang X, Lan J, Shen J, Wang S. Yang F, Wang Y, Li Y, Chen D. Progranulin improves neural development via the PI3K/Akt/GSK-3B?pathway in the cerebellum of a VPA-induced rat model of ASD. Transl Psychiatry. 12: 114, 2022
73. Kim W E, Yue B, Serrero G. Signaling Pathway of GP88 (Progranulin) in Breast Cancer Cells: Upregulation and Phosphorylation of c-myc by GP88/Progranulin in Her2-Overexpressing Breast Cancer Cells. Breast Cancer (Aucki). 9(Suppl 2): 71-77, 2016.
74. Gao X, Joselin A P, Wang L, Kar A, Ray P, Bateman A, Goate A M, Wu J Y. Progranulin promotes neurite outgrowth and neuronal differentiation by regulating GSK-3β. Protein Cell 1: 552, 2010-
75. Almeida S, Zhang Z, Coppola G, Mao W, Futai K, Kary das A, Geschwind M D, Tartaglia M C, Gao F, Gianni D, Sena-Esteves M. Geschwind D I1, Miller B L, Farese R V Jr, Gao F B. Induced pluripotent stem cell models of progranulin-deficient frontotemporal dementia uncover specific reversible neuronal defects. Cell Rep 25; 2(4):789-798, 2012.
76. Chitramuthu B. Bateman A. Progranulin and the receptor ty rosine kinase EphA2, partners in crime? J Cell Biol. 2016:215(5):603-605. doi:10.1083/jcb.201610097
77. Altmann C, Vasic V, Hardt S, Heidler J, Haussler A, Wittig I, Schmidt M H H, Tegeder 1. Progranulin promotes peripheral nerve regeneration and reinnervation: role of notch signaling. Mol Neurodegener. 11(1):69, 2016
78. Chen R, Kim O, Yang J. Sato K. Eisenmann K M, McCarthy J, Chen H. Qiu Y. Regulation of Akt/PKB activation by tyrosine phosphorylation. J Biol Chem. 276: 31858-31862, 2001.
79. Tang W, Lu Y, Tian Q-Y et al., The growth factor progranulin binds to TNF receptors and is therapeutic against inflammatory arthritis in mice. Sci 332: 478-484, 201
80. Nguyen A D, Nguyen T A, Martens L H, Farese R V, Jr. Progranulin: at the interface of neurodegenerative and metabolic diseases. Trends Endocrinol Metab 24(12): 10.1016/jtem.2013.08.00s.
81. Sajan M P, Ivey R A, III, Farese R V. Metformin action in human hepatocytes. Co-activation of atypical protein kinase C alters 5′-AMP-activated protein kinase effects on lipogenic and gluconeogenic enzyme expression. Diabetologia 56: 2507-3010, 2013
82. Sajan M P, Lee M C, Foufelle F, et al. Coordinated regulation of hepatic FoxO1, PGC-1□ and SREBP-1c facilitates insulin action and resistance. Cellular Signalling 43:62-70,
83. Apostolatos A H, Apostolatos C A, Ratnayake W S, et al. Preclinical testing of 5-amino-1-((1R,2S,3S,4R)-2,3-dihydroxy-4-methylcyclopentyl)-1H-imidazole-4-carboxamide: a potent protein kinase C-t inhibitor as a prostate carcinoma therapeutic. Anticancer Drugs 30: 65, 2019
84. Erdogan E, Lemark T, Stalling-Mans M, et al. Aurothiomalate inhibits transformational growth by targeting the PB1 domain of protein kinase Ci. J Biol Chem 281: 28450-28459, 2006.
85. Moscat J, Diaz-Meco M T, Wooten M W. Of the atypical PKCs, Par-4 and p62: recent understandings of the biology and pathology of a PB1-dominated complex. Cell death and Differentiation 16: 1426-1437, 2009
86. Moscat J, Diaz-Meco M T. The atypical protein kinase Cs. EMBO Rep 1: 399-403, 2000.
87. Diaz-Mecco M T, Municio M M, Frutos S, et al. The product of Par-4, a gene induced during apoptosis, interacts selectively with isoforms of aPKC. Cell 86: 777-786, 1996
88. Madeira J M, Renschler C J, Mueller B, et al. Novel protective properties of auronafin: inhibition of human astrocyte cytotoxic secretions and direct neuroprotection. Life Sci 92: 1072, 2013
89. Sacktor T C. PKMzeta, LTP maintenance, and the dynamic molecular biology of memory storage. Prog Brain Res 169: 27-40, 2008
90. Tsokas P, Hsieh C, Yao Y, Sajan M P, Farese R V, Sacktor T C, et al. Compensation for PKM□ in LTP and spatial long-term memory in mutant mice. eLife Sci 5: e14846, 2016:
91. Sheng T, Wang S, Qian D, Gao J, Ohno S, Lu W. Learning-induced suboptimal compensation for PKC function in mutant mice. Cerebral Cortex 27:3284-3293, 2017
92. Nguyen A D, Nguyen T A, Zhang J et al. Murine knockin model for progranulin-deficient frontotemporal dementia with nonsense mediated mRNA decay. Proc Natl Acad Sci USA 115: E-2849E2858; 2018
93. Krabbe G, Minami S S, EtchegarayJI, et al. Microglial NFkB-TNFa hyperactivation induces obsessive-compulsive behavior in mouse models of progranulin-deficient frontotemporal dementia. Proc Natl Acad Sci 114: 5029-5034
94. Arrant A E, Onyilo V C, Unger D E, Roberson E D. Progranulin therapy inproves lysosomal dysfunction and microglial pathology associated with frontotemporal dementia and neuronal ceroid lipofuscinosis. J Neurosci 38: 2341-2358, 2018
95. Arrant A E, Filiano A J, Unger D E, Young A H, Roberson E D. Restoring neuronal progranulin reverses deficits in a mouse model of frontotemporal dementia. Brain 140: 1447-1465, 2017
96. Filiano A J, Martens L H, Young A H et al. Dissociation of FTD-Related Deficits and Neuroinflammation in Progranulin Haploinsufficient Mice. J Neurosci 33: 5352-5361, 2013
97. Sajan M P, Lee M C, Foufelle F, et al. Coordinated regulation of hepatic Fox01, PGC-1 and SREBP-1c facilitates insulin action and resistance. Cellular Signalling 43:62-70, 2018
98. Farese R V, Sajan M P, Yang H, et al. Muscle-specific Knockout of Protein Kinase C Impairs Glucose Transport and Induces Metabolic/Diabetic Syndromes J Clin Invest 117: 2289, 2007
99. Sajan M P, Jurzak M J, Samuels V T, Shulman G I, Braun U, Leitges M, Farese R V. Impairment of insulin-stimulated glucose transport and ERK activation by adipocyte-specific knockout of PKC-λ/ι produces a phenotype characterized by diminished adiposity and enhanced insulin suppression of hepatic gluconeogenesis. Adipocyte 3:19-29, 201481.
100. Knight, E. M. Martins, I V., Gimasgoz, S., Allan, S. M., & Lawrence. C. B. (2014). High-fat diet-induced memory impairment in triple-transgenic Alzheimer's disease (3×TgAD) mnice is independent of changes in amyloid and tau pathology. Neurobiology of Aging. 35(8), 1821-1832. https://doi.org/10.1016/j.neurobiolaging.2014.02.010
101. Luo M, Langlais P, Yi Z, Lefort N, De Filippis E A, Hwang H, Christ-Roberts C Y, Mandarino L J. Phosphorylation ofhuman insulin receptor substrate-1 at Serine 629 plays a positive role in insulin signaling. Endocrinology. 2007 October; 148(10):4895-905. doi: 10.1210/en.2007-0049. Epub 2007 Jul. 19.
102. Werner E D, Lee J, Hansen L. Yuan M, Shoelson S E. Insulin resistance due to phosphorylation of insulin receptor substrate-1 at serine 302. J Biol Chem. 2004 Aug. 20; 279(34):35298-305. doi: 10.1074/jbc.M405203200. Epub 2004 Jun. 14. PMID: 15199052.
103. Hotamisligil G S, Peraldi P, Budavari A, Ellis R, White M F, Spiegelman B M. IRS-1-mediated inhibition of insulin receptor tyrosine kinase activity in TNF-alpha- and obesity-induced insulin resistance. Science. 1996 Feb. 2; 271(5249):665-8. doi: 10.1126/science.271.5249.665. PMID:

### SEQUENCE LISTING

(ZIP)
SEQ ID NO: 1
SIYRRGARRWRKL

We claim:

1. A method of treating a neurodegenerative disorder in a subject in need thereof, the method comprising:
administering an effective amount of a PKC inhibitor, wherein the PKC inhibitor is capable of crossing the blood brain barrier, and wherein the administered PKC inhibitor decreases BACE1 levels in the subject in need thereof.

2. (canceled)

3. A method of treating a neurodegenerative disorder in a subject in need thereof, the method comprising:
administering an effective amount of a PKC inhibitor, wherein the PKC inhibitor is capable of crossing the blood brain barrier, and wherein the administered PKC inhibitor increases a functional ability of the subject.

4. The method of claim 3, wherein the functional ability is determined by at least one test from the group comprising memory test, learning test, spatial memory test, object location memory test, and visual memory test.

5. The method of claim 1, wherein the BACE1 levels are protein levels or RNA levels.

6.-8. (canceled)

9. The method of claim 5, wherein the BACE1 levels are measured in the brain or liver and are compared to a control, wherein the control is a sample from a healthy subject, a sample previously collected from the subject in need thereof, or an established expression level.

10.-12. (canceled)

13. The method of claim 1, wherein the BACE1 levels are measured after the PKC inhibitor has been administered for a duration of 1 to 31 days.

14. The method of claim 13, wherein the BACE1 levels are measured after the PKC inhibitor has been administered for a duration of 1 day or 21 days.

15. (canceled)

16. The method of claim 1, wherein the PKC inhibitor is an atypical PKC inhibitor.

17. The method of claim 16, wherein the atypical PKC inhibitor is selected from the group comprising auranofin, ICAP, ACPD, ICA-1, DNDA, and ζ-Stat.

18. The method of claim 17, wherein the atypical PKC inhibitor is auranofin.

19. The method of claim 17, wherein the atypical PKC inhibitor is ICAP, and wherein the ICAP is administered at a dosage of between 1 to 5000 mg per kg of body weight.

20. (canceled)

21. The method of claim 19, wherein ICAP is given at a dosage of 200 mg per kg of body weight.

22. The method of claim 1, wherein the PKC inhibitor is administered intravenously, orally, nasally, or subcutaneously.

23. (canceled)

24. (canceled)

25. The method of claim 1, wherein the neurodegenerative disorder is Alzheimer's disease.

26. The method of claim 1, wherein the subject has insulin resistance, obesity, metabolic syndrome, or type 2 diabetes.

27. The method of claim 26, wherein the subject is currently taking insulin.

28. The method of claim 1, wherein the subject has hyperinsulinemia.

29. The method of claim 1, wherein the PKC inhibitor decreases proinflammatory cytokine levels.

30. The method of the claim 29, wherein the proinflammatory cytokine is TNF-α or IL-6.

31. A method of treating Alzheimer's Disease in a subject in need thereof, the method comprising:
nasally administering auranofin to the subject in need thereof.
